(19) United States
(12) Patent Application Publication
(10) Pub. No.: US 2026/0193318 A1
(43) Pub. Date: Jul. 9, 2026

(54) CHIMERIC CYTOKINE RECEPTORS BEARING A PD-1 ECTODOMAIN

(71) Applicant: Allogene Therapeutics, Inc., South San Francisco, CA (US)

(72) Inventors: Regina Junhui LIN, San Mateo, CA (US); Thomas John VAN BLARCOM, Oakland, CA (US); Siler PANOWSKI, Berkeley, CA (US); Barbra Johnson SASU, San Francisco, CA (US)

(73) Assignee: Allogene Therapeutics, Inc., South San Francisco, CA (US)

(21) Appl. No.: 19/551,160

(22) Filed: Feb. 26, 2026

Related U.S. Application Data
(63) Continuation of application No. 18/365,014, filed on Aug. 3, 2023.
Application No. 18/365,014 is a division of application No. 16/804,545, filed on Feb. 28, 2020, now Pat. No. 11,786,553.
(60) Provisional application No. 62/812,799, filed on Mar. 1, 2019.
(60) Provisional application No. 62/894,659, filed on Aug. 30, 2019.
(60) Provisional application No. 62/980,737, filed on Feb. 24, 2020.

(51) Int. Cl. C07K 14/715; A61K 38/00; A61K 40/11; A61K 40/31; A61K 40/42; C07K 16/28; C07K 16/30; C12N 5/0783; C12N 15/67

(52) U.S. Cl. CPC C07K 14/7155; A61K 40/11; A61K 40/31; A61K 40/4204; A61K 40/421; C07K 16/2818; C07K 16/30; C12N 5/0636; C12N 15/67; A61K 38/00; A61K 2239/17; A61K 2239/23; C07K 2319/03; C07K 2319/33; C07K 2319/74

(57) ABSTRACT

Provided herein are PD-1 chimeric cytokine receptors. When present on chimeric antigen receptor (CAR)-bearing immune cells, such receptors allow for increased immune cell activation, proliferation, persistence, and/or potency, when engaged with PD-1 ligands or activation with an anti-PD-1 antibody. Also provided are methods of making and using the chimeric cytokine receptors described herein.

### CROSS REFERENCE TO RELATED APPLICATIONS

This application is a continuation of U.S. application Ser. No. 18/365,014, filed Aug. 3, 2023, which application is a divisional of U.S. application Ser. No. 16/804,545, filed Feb. 28, 2020, which claims the benefit of priority to U.S. Provisional Application No. 62/812,799, filed on Mar. 1, 2019; U.S. Provisional Application No. 62/894,659, filed on Aug. 30, 2019; and U.S. Provisional Application No. 62/980,737, filed on Feb. 24, 2020, the contents of all of which are hereby incorporated by reference in their entireties.

### SEQUENCE LISTING

The instant application contains a Sequence Listing which has been submitted electronically in XML format and is hereby incorporated by reference in its entirety. Said XML copy, created on Jul. 31, 2023, is named AT-024_05_SL.xml and is 341,206 bytes in size.

### BACKGROUND

Adoptive transfer of immune cells (e.g. T-cells) genetically modified to recognize malignancy-associated antigens is showing promise as a new approach to treating cancer. For example, T-cells can be genetically modified to express chimeric antigen receptors (CARs), which are fusion proteins comprised of an antigen recognition moiety and T-cell activation domains.

T-cell proliferation, cytotoxic potency and persistence is driven by signal transduction pathways. Conventional CAR designs provide two signals—CD3zeta activation (Signal 1) and co-stimulation (Signal 2, e.g. via 4-1BB, OX40, and/or CD28 expression). In some contexts, a third signal (Signal 3), cytokine-induced cytokine receptor signaling (e.g. cytokine support for immune potentiation), may be desirable. Approaches to provide Signal 3 have however been met with significant limitations.

One approach to provide cytokine support includes combining CAR-T-cell therapy with systemic infusions of recombinant cytokines/cytokine mimetics, and engineering CAR-T-cells to secrete/express cytokines extracellularly. As cytokines have pleiotropic effects and can also impact the function of other cell types, the systemic administration or production of immune-potentiating cytokines by CAR-T-cells have at least two major drawbacks: (i) these approaches can cause systemic toxicity in humans, and (ii) in the context of allogeneic CAR-T-cell therapy, these approaches may cause bystander host immune-activation that could accelerate the rejection of allogeneic CAR-T-cells, thereby compromising therapeutic efficacy. Another approach to provide cytokine support was based on introducing a constitutively activated dimerized cytokine receptor, an IL-7Ra—this limits the nature (IL-7 signaling only) and magnitude of signaling output. Yet another approach to provide cytokine support involved incorporating Signal 3 directly into the CAR molecule (Nat Med. 2018 March; 24(3):352-359.). A limitation of this approach is that the strength of Signal 3 dependent on the strength of CAR activation. In the absence of target (and CAR activation), Signal 3 would not be transduced.

Needed are solutions to circumvent these drawbacks by targeting cytokine signals specifically to CAR-T-cells in a context-dependent manner, thus allowing for an improved safety profile and therapeutic efficacy. Provided herein and compositions and methods that address this need.

### SUMMARY

Provided herein are inducible PD-1 chimeric cytokine receptors, active, for example, when engaged with PD-1 ligands or activated with an anti-PD-1 antibody. When present on chimeric antigen receptor (CAR)-bearing immune cells, and engaged with PD-1 ligands and/or activated with an anti-PD-1 antibody, such receptors allow for increased cytokine receptor signaling (Signal 3), leading to increased immune cell activation, proliferation, persistence, and/or potency of the CAR-bearing immune cells. Accordingly, the PD-1 chimeric cytokine receptors of the disclosure allow for cytokine signals to be transmitted into the immune cell with endogenous PD-1 ligands (PD-L1, PD-L2), whereby blocking their immune-suppressive signals, and converting them into immune-potentiating signals that can work in concert with, or synergize, CAR-driven activity. Moreover, as clinically approved anti-PD-1 antibodies can cluster and activate the PD-1 chimeric cytokine receptors of the disclosures, patients treated with anti-PD-1 may benefit not only from the blockage of the endogenous PD-1 signaling, but from also the activation of cytokine signaling in cells bearing the PD-1 chimeric cytokine receptors.

In some embodiments, provided herein are constitutively active PD-1 chimeric cytokine receptors. When present on chimeric antigen receptor (CAR)-bearing immune cells, these constitutively active receptors allow for increased basal cytokine receptor signaling (Signal 3). In some embodiments, the activity of the constitutively active PD-1 chimeric cytokine receptors can be increased when engaged with PD-1 ligands or activated with an anti-PD-1 antibody.

Accordingly, in one aspect, provided herein is a PD-1 chimeric cytokine receptor comprising: (a) a PD-1 ectodomain; (b) a transmembrane domain; (c) a Janus Kinase (JAK)-binding domain; and (d) a recruiting domain. The PD-1 ectodomain may comprise a portion of the extracellular region of a PD-1 protein, or may comprise a PD-1 ligand antigen binding domain.

In a related aspect provided herein is a polynucleotide encoding any one of the chimeric cytokine receptors of the disclosure, and an expression vector comprising such polynucleotide. In some embodiments, the polynucleotide further encodes for a chimeric antigen receptor (CAR), wherein the CAR binds to a target of interest. The target of interest can be any molecule of interest, including, for example, without limitation any one or more of those presented in Table 11.

In another aspect, provided herein is an engineered immune cells comprising at least one chimeric antigen receptor (CAR) and at least one chimeric cytokine receptor of the disclosure. In some embodiments the immune cell is a T-cell. In some embodiments the immune cell is an allogeneic immune cell. In other embodiments, the immune cell is an autologous immune cell. The immune cell may be selected from the group consisting of: T-cell, dendritic cell, killer dendritic cell, mast cell, NK-cell, macrophage, monocyte, B-cell and an immune cell derived from a stem cell. In a related aspect, provided herein is a pharmaceutical composition comprising any of the engineered immune cells of the disclosure, and a kit comprising such a pharmaceutical composition.

In another aspect, provided herein is a method of treating a cancer in a subject, comprising administering to the subject a therapeutically effective amount of any of the engineered immune cells described herein.

### BRIEF DESCRIPTION OF THE DRAWINGS

FIG. 1A shows a schematic of the certain embodiments of the PD-1 chimeric cytokine receptors of the disclosure.

FIG. 1B shows a schematic of the PD-1 chimeric cytokine receptor of the disclosure with a wild type PD-1 ectodomain. FIG. 1B also shows a schematic of the PD-1 chimeric cytokine receptor—CAR vector

FIG. 1C shows a schematic of an exemplary PD-1 chimeric cytokine receptor of the disclosure.

FIG. 1D shows a schematic of the PD-1 chimeric cytokine receptor—CAR vector, and the control FKBP chimeric cytokine receptor CAR vector.

FIGS. 2A-2D show Stat reporter activity of the PD-1 chimeric cytokine receptor in response to PD-L1-Fc (FIGS. 2A-B) and anti-PD-1 (FIGS. 2C-D).

FIG. 3 shows CAR and PD-1 chimeric cytokine receptor co-expression on the Day 14 following transduction of the CAR and the PD-1 chimeric cytokine receptor.

FIGS. 4A-4B show phospho-Stat5 staining in human CAR-T-cells following the treatment with PD-L1-Fc (FIG. 4A) or anti-PD-1 (FIG. 4B).

FIGS. 5A-5D show a schematic of PD-1 chimeric cytokine receptors with various ectodomains engineered for enhanced avidity or affinity. FIG. 5A shows the prototypic WT PD-1 chimeric cytokine receptor bearing the TpoR (478-582) TM/JAK2-activating domain; FIG. 5B shows the 2×PD-1 chimeric cytokine receptor bearing two WT PD-1 ectodomains in tandem; FIG. 5C shows the 3×PD-1 chimeric cytokine receptor bearing three WT PD-1 ectodomains in tandem; FIG. 5D shows the high affinity (HA) PD-1 chimeric cytokine receptor with ectodomain mutations.

FIGS. 6A-6D show the response of PD-1 chimeric cytokine receptors bearing ectodomain modifications following treatment with PD-L1-Fc (FIGS. 6A-6B) and anti-PD-1 (FIGS. 6C-D).

FIGS. 7A-7B show results from the cell binding assay between PD-1 chimeric cytokine receptor CAR-T-cells and PD-L1-Fc (FIG. 7A) or clinically-approved anti-PD-1 antibodies (FIG. 7B).

FIGS. 8A-8B show the response of CAR-T-cells bearing PD-1 chimeric cytokine receptor ectodomain variants following treatment with PD-L1-Fc (FIG. 8A) or anti-PD-1 (FIG. 8B).

FIGS. 9A-9B show the cytotoxic response of the PD-1 chimeric cytokine receptor CAR-T-cells against target cells expressing PD-1 ligands.

FIGS. 10A-10B show the amino acid sequences for the wild type TpoR and the various TM deletion (FIG. 10a) or insertion (FIG. 10b) variants.

FIGS. 11A-11B show the response of a HA PD-1 chimeric cytokine receptor bearing TpoR TM deletions (FIG. 11A) and extensions (FIG. 11B) following treatment with PD-L1-Fc.

FIGS. 12A-12B shows the response of HA PD-1 chimeric cytokine receptor bearing TpoR TM deletions (FIG. 12A) and extensions (FIG. 12B) following treatment with anti-PD-1 (Nivolumab).

FIGS. 13A-13B show the cytotoxic response of PD-1 chimeric cytokine receptor CAR-T-cells in the absence (FIG. 13A) or presence (FIG. 13B) of anti-PD-1.

FIG. 14 shows the cytotoxic response of CAR-T-cells coexpressing either a DN WT PD-1, a DN HA PD-1 or the HA PD-1 chimeric cytokine receptor.

FIG. 15 shows a Stat reporter activity of the constitutively active, dominant negative, and inducible PD-1 chimeric cytokine receptors in response to PD-L1-Fc and crosslinked PD-L1-Fc.

FIG. 16 shows the cytotoxic activity of HA PD1 switch CAR T cells with the indicated TpoR TM domains at an E:T ratio of 1:4 and in the absence of anti-PD-1.

FIGS. 17A-17E show the cytotoxic activity of HA PD1 switch CAR T cells with the indicated TpoR TM domains at an E:T ratio of 1:8, and in the absence or presence of anti-PD-1.

### DETAILED DESCRIPTION

Provided herein are inducible PD-1 chimeric cytokine receptors, active when engaged with PD-1 ligands or activated with an anti-PD-1 antibody. Also provided herein are constitutively active PD-1 chimeric cytokine receptors. Provided herein are chimeric antigen receptor (CAR)-bearing immune cells (CAR-I cells, e.g. CAR-T-cells), expressing the PD-1 chimeric cytokine receptors of the disclosure. Also provided herein are methods of making and using the PD-1 chimeric cytokine receptors.

### I. PD-1 Chimeric Cytokine Receptors

The inducible PD-1 chimeric cytokine receptors of the disclosure activate signaling upon, for example, binding a PD-1 ligand (e.g. PD-L1, PD-L2), or a PD-1 antibody. These receptors activate signaling when monomers of the receptor cluster and/or dimerize. In some embodiments, a monomer of the PD-1 chimeric cytokine receptor of the disclosure comprises: (a) a PD-1 ectodomain; (b) a transmembrane domain; (c) a Janus Kinase (JAK)-binding domain; and; (d) a STAT-recruiting domain (e.g. from the cytoplasmic domain of a receptor; e.g. from a cytokine receptor). In some embodiments, a monomer of the PD-1 chimeric cytokine receptor of the disclosure comprises: (a) a PD-1 ectodomain; (b) a transmembrane domain; (c) a Janus Kinase (JAK)-binding domain; and; (d) a recruiting domain (e.g. from the cytoplasmic domain of a receptor; e.g. from a cytokine receptor). The recruiting domain can be a STAT-recruiting domain, an AP1-recruiting domain, a Myc/Max-recruiting domain; or a NFkB-recruiting domain. The PD-1 chimeric cytokine receptors can bind to PD-1 ligands, and/or are clustered and activated with an anti-PD-1 antibody. The PD-1 chimeric cytokine receptors activate signaling upon for example binding a PD-L1 ligand, PD-L2 ligand, and/or a PD-1 antibody.

The constitutively active PD-1 chimeric cytokine receptors of the disclosure are active regardless of PD-1 ligand availability, but may increase activity in the presence of a PD-ligand. In some embodiments, a monomer of the constitutively active PD-1 chimeric cytokine receptor of the disclosure comprises: (a) a PD-1 ectodomain; (b) a transmembrane domain; (c) a Janus Kinase (JAK)-binding domain; and; (d) a STAT-recruiting domain (e.g. from the cytoplasmic domain of a receptor; e.g. from a cytokine receptor). In some embodiments, a monomer of the constitutively active PD-1 chimeric cytokine receptor of the disclosure comprises: (a) a PD-1 ectodomain; (b) a transmembrane domain; (c) a Janus Kinase (JAK)-binding domain; and; (d) a recruiting domain (e.g. from the cytoplasmic domain of a receptor; e.g. from a cytokine receptor). The recruiting domain can be a STAT-recruiting domain, an AP1-recruiting domain, a Myc/Max-recruiting domain; or a NFkB-recruiting domain. In some embodiments, constitutively active PD-1 chimeric cytokine receptors can bind to PD-1 ligands or an anti-PD-1 antibody, and/or are clustered and the activity of the receptors can be further increased by engagement with PD-1 ligands or an anti-PD-1 antibody.

### A. PD-1 Ectodomains

The PD-1 chimeric cytokine receptors of the disclosure comprise a PD-1 ectodomain. The PD-1 ectodomain is the domain of the chimeric cytokine receptor that extends into the extracellular space. PD-1 ectodomains of the disclosure are capable of binding PD-1 ligands (e.g. PD-L1, PD-L2) and anti-PD-1 antibodies, leading to binding-induced signal transduction. As contemplated herein, the term “PD-1 ectodomain” comprises at least a portion of the extracellular domain of a PD-1 protein or comprises a PD-1 ligand antigen binding domain (e.g. a PD-L1 antigen binding domain, or a PD-L2 antigen binding domain).

In some embodiments, the PD-1 ectodomain comprises a portion of a wild type PD-1. In some embodiments, the PD-1 ectodomain comprises mutations to the wild-type PD-1. In some embodiments, the PD-1 ectodomain comprises repeats of wild-type and/or mutated PD-1 amino acid sequences.

In some embodiments, the PD-1 ectodomain comprises a PD-1 ligand antigen binding domain, for example, the antigen binding domain of an anti-PD-L1 antibody or an anti-PD-L2 antibody. In some embodiments, the antigen binding domain is an scFv. In some embodiments, the antigen binding domain is a single chain antibody. In some embodiments, the antigen binding domain comprises a Fab portion of a PD-1 ligand antibody.

Table 1 shows exemplary PD1 amino acid ectodomain sequences of the disclosure. It is noted that the expression and extracellular location of the exemplary PD1 amino acid sequences can be achieved with the use of a signal sequence. In an exemplary embodiment, a CD8 signal sequence (CD8SS) MALPVTALLLPLALLLHAARP (SEQ ID NO: 1) is utilized.

TABLE 1
Exemplary Ectodomain Sequences

| | | SEQ |
|---|---|---|
| Ectodomain | Amino acid sequence | ID NO: |
| Wild type (WT) PD1 | PGWFLDSPDRPWNPPTFSPALLVVTEGDNATFTC | 2 |
| ectodomain (PD-1(21- | SFSNTSESFVLNWYRMSPSNQTDKLAAFPEDRSQ | |
| 207)) | PGQDCRFRVTQLPNGRDFHMSVVRARRNDSGTY | |
| | LCGAISLAPKAQIKESLRAELRVTERRAEVPTAHP | |
| | SPSPRPAGQFQTLV | |
| | VGVVGGLLGSLVLLVWVLAVICSRAARGTIGAR | |
| | RTGQ | |
| 2x WTPD1 ectodomain | PGWFLDSPDRPWNPPTFSPALLVVTEGDNATFTC | 3 |
| (PD-1(21-207)-PD-1(21- | SFSNTSESFVLNWYRMSPSNQTDKLAAFPEDRSQ | |
| 207)) | PGQDCRFRVTQLPNGRDFHMSVVRARRNDSGTY | |
| | LCGAISLAPKAQIKESLRAELRVTERRAEVPTAHP | |
| | SPSPRPAGQFQTLVVGVVGGLLGSLVLLVWVLA | |
| | VICSRAARGTIGARRTGQPGWFLDSPDRPWNPPT | |
| | FSPALLVVTEGDNATFTCSFSNTSESFVLNWYRM | |
| | SPSNQTDKLAAFPEDRSQPGQDCRFRVTQLPNGR | |
| | DFHMSVVRARRNDSGTYLCGAISLAPKAQIKESL | |
| | RAELRVTERRAEVPTAHPSPSPRPAGQFQTLVVG | |
| | VVGGLLGSLVLLVWVLAVICSRAARGTIGARRT | |
| | GQ | |
| 3x WTPD1 ectodomain | PGWFLDSPDRPWNPPTFSPALLVVTEGDNATFTC | 4 |
| (PD-1(21-207)-PD-1(21- | SFSNTSESFVLNWYRMSPSNQTDKLAAFPEDRSQ | |
| 207)-PD-1(21-207)) | PGQDCRFRVTQLPNGRDFHMSVVRARRNDSGTY | |
| | LCGAISLAPKAQIKESLRAELRVTERRAEVPTAHP | |
| | SPSPRPAGQFQTLVVGVVGGLLGSLVLLVWVLA | |
| | VICSRAARGTIGARRTGQPGWFLDSPDRPWNPPT | |
| | FSPALLVVTEGDNATFTCSFSNTSESFVLNWYRM | |
| | SPSNQTDKLAAFPEDRSQPGQDCRFRVTQLPNGR | |
| | DFHMSVVRARRNDSGTYLCGAISLAPKAQIKESL | |
| | RAELRVTERRAEVPTAHPSPSPRPAGQFQTLVVG | |
| | VVGGLLGSLVLLVWVLAVICSRAARGTIGARRT | |
| | GQPGWFLDSPDRPWNPPTFSPALLVVTEGDNATF | |
| | TCSFSNTSESFVLNWYRMSPSNQTDKLAAFPEDR | |
| | SQPGQDCRFRVTQLPNGRDFHMSVVRARRNDSG | |
| | TYLCGAISLAPKAQIKESLRAELRVTERRAEVPTA | |
| | HPSPSPRPAGQFQTLVVGVVGGLLGSLVLLVWV | |
| | LAVICSRAARGTIGARRTGQ | |
| High-affinity (HA) PD1 | PGWFLDSPDRPWNPPTFSPALLVVTEGDNATFTC | 5 |
| ectodomain | SFSNTSESFHVIWHRESPSGQTDTLAAFPEDRSQP | |
| | GQDCRFRVTQLPNGRDFHMSVVRARRNDSGTY | |
| | VCGVISLAPKIQIKESLRAELRVTERRAEVPTAHP | |
| | SPSPRPAGQFQTLVVGVVGGLLGSLVLLVWVLA | |
| | VICSRAARGTIGARRTGQ | |
| FKBP(F36V) | MGVQVETISPGDGRTFPKRGQTCVVHYTGMLED | 6 |
| | GKKVDSSRDRNKPFKFMLGKQEVIRGWEEGVA | |
| | QMSVGQRAKLTISPDYAYGATGHPGIIPPHATLV | |
| | FDVELLKLE | |
| Wildtype (WT) PD1 | PGWFLDSPDRPWNPPTFSPALLVVTEGDNATFTC | 132 |
| ectodomain (PD-1(21- | SFSNTSESFVLNWYRMSPSNQTDKLAAFPEDRSQ | |
| 170)) | PGQDCRFRVTQLPNGRDFHMSVVRARRNDSGTY | |
| | LCGAISLAPKAQIKESLRAELRVTERRAEVPTAHP | |
| | SPSPRPAGQFQTLV | |
| High-affinity (HA) PD1 | PGWFLDSPDRPWNPPTFSPALLVVTEGDNATFTC | 133 |
| ectodomain (21-170) | SFSNTSESFHVIWHRESPSGQTDTLAAFPEDRSQP | |
| | GQDCRFRVTQLPNGRDFHMSVVRARRNDSGTY | |
| | VCGVISLAPKIQIKESLRAELRVTERRAEVPTAHP | |
| | SPSPRPAGQFQTLV | |
| 2x WTPD1 ectodomain | PGWFLDSPDRPWNPPTFSPALLVVTEGDNATFTC | 168 |
| (PD-1(21-170)-G-PD- | SFSNTSESFVLNWYRMSPSNQTDKLAAFPEDRSQ | |
| 1(21-170)) | PGQDCRFRVTQLPNGRDFHMSVVRARRNDSGTY | |
| | LCGAISLAPKAQIKESLRAELRVTERRAEVPTAHP | |
| | SPSPRPAGQFQTLVGPGWFLDSPDRPWNPPTFSP | |
| | ALLVVTEGDNATFTCSFSNTSESFVLNWYRMSPS | |
| | NQTDKLAAFPEDRSQPGQDCRFRVTQLPNGRDF | |
| | HMSVVRARRNDSGTYLCGAISLAPKAQIKESLRA | |
| | ELRVTERRAEVPTAHPSPSPRPAGQFQTLV | |
| 3x WTPD1 ectodomain | PGWFLDSPDRPWNPPTFSPALLVVTEGDNATFTC | 169 |
| (PD-1(21-170)-G-PD- | SFSNTSESFVLNWYRMSPSNQTDKLAAFPEDRSQ | |
| 1(21-170)-G-PD-1(21- | PGQDCRFRVTQLPNGRDFHMSVVRARRNDSGTY | |
| 170)) | LCGAISLAPKAQIKESLRAELRVTERRAEVPTAHP | |
| | SPSPRPAGQFQTLVGPGWFLDSPDRPWNPPTFSP | |
| | ALLVVTEGDNATFTCSFSNTSESFVLNWYRMSPS | |
| | NQTDKLAAFPEDRSQPGQDCRFRVTQLPNGRDF | |
| | HMSVVRARRNDSGTYLCGAISLAPKAQIKESLRA | |
| | ELRVTERRAEVPTAHPSPSPRPAGQFQTLVGPGW | |
| | FLDSPDRPWNPPTFSPALLVVTEGDNATFTCSFSN | |
| | TSESFVLNWYRMSPSNQTDKLAAFPEDRSQPGQ | |
| | DCRFRVTQLPNGRDFHMSVVRARRNDSGTYLCG | |
| | AISLAPKAQIKESLRAELRVTERRAEVPTAHPSPS | |
| | PRPAGQFQTLV | |

In some embodiments, the PD-1 ectodomain comprises a wild type PD-1 ectodomain.

In some embodiments, the PD-1 ectodomain comprises a wild type PD-1 and comprises the amino acid sequence of SEQ ID NO: 2. In some embodiments, the PD-1 ectodomain comprises a wild type PD-1 and comprises the amino acid sequence of SEQ ID NO: 132.

In some embodiments, the PD-1 ectodomain comprises mutations to the wild type PD-1 ectodomain sequence. In some embodiments, the PD-1 ectodomain sequence is a high affinity PD-1 ectodomain. In some embodiments, the PD-1 ectodomain sequence is a high affinity PD-1 ectodomain and comprises the amino acid sequence of SEQ ID NO: 5. In some embodiments, the PD-1 ectodomain sequence is a high affinity PD-1 ectodomain and comprises the amino acid sequence of SEQ ID NO: 133.

In some embodiments, the PD-1 ectodomain comprises a tandem arrangement of greater than one wild type PD-1 ectodomain sequence, for example at least two, at least three, at least four, or at least five wild type PD-1 ectodomain sequences. In some embodiments, the PD-1 ectodomain comprises a tandem arrangement of two wild type PD-1 ectodomain sequences and comprises the amino acid sequence of SEQ ID NO: 3. In some embodiments, the PD-1 ectodomain comprises a tandem arrangement of two wild type PD-1 ectodomain sequences and comprises the amino acid sequence of SEQ ID NO: 168. In some embodiments, the PD-1 ectodomain comprises a tandem arrangement of three wild type PD-1 ectodomain sequences and comprises the amino acid sequence of SEQ ID NO: 4. In some embodiments, the PD-1 ectodomain comprises a tandem arrangement of three wild type PD-1 ectodomain sequences and comprises the amino acid sequence of SEQ ID NO: 169.

In some embodiments, the PD-1 ectodomain comprises a tandem arrangement of greater than one high affinity PD-1 ectodomain sequence, for example at least two, at least three, at least four, or at least five high affinity PD-1 ectodomain sequences.

In some embodiments, the PD-1 ectodomain comprises a high affinity PD-1 and comprises the amino acid sequence of SEQ ID NO: 5. In some embodiments, the high affinity PD-1 ectodomain comprises the amino acid sequence of SEQ ID NO: 133. In some embodiments, the PD-1 ectodomain comprises a tandem arrangement of two high affinity PD-1 ectodomain sequences. In some embodiments, the PD-1 ectodomain comprises a tandem arrangement of three high affinity PD-1 ectodomain sequences.

In some embodiments, the PD-1 ectodomain comprises a tandem arrangement of a combination of wild type and high affinity PD-1 ectodomain sequences, for example a combination of at least two, at least three, at least four, or at least five wild type and high affinity PD-1 ectodomain sequences.

In some embodiments, the greater than one wide type PD-1 ectodomain, or greater than one high affinity PD-1 ectodomain, or a combination thereof, can be connected either directly, or via a linker. In some embodiments, the linker can comprise one or more amino acid residues. In some embodiments, the linker is a Gly.

In some embodiments, the PD-1 ectodomain is a dominant negative. Table 2 shows exemplary PD-1 dominant negative (DN) sequences of the disclosure. The DN sequences of Table 2 may be expressed with the aid of a signal sequence, e.g. a CD8SS signal sequence of SEQ ID NO: 1

TABLE 2
PD1 dominant negative (DN) sequences:

| | | SEQ |
|---|---|---|
| PD1 DN | Amino acid sequence | ID NO: |
| Dominant | PGWFLDSPDRPWNPPTFSPALLVVTEGDNATFTCSFSNTSESF | 7 |
| negative wild | VLNWYRMSPSNQTDKLAAFPEDRSQPGQDCRFRVTQLPNGR | |
| type PD1 | DFHMSVVRARRNDSGTYLCGAISLAPKAQIKESLRAELRVTE | |
| (21-207) | RRAEVPTAHPSPSPRPAGQFQTLVVGVVGGLLGSLVLLVWV | |
| | LAVICSRAARGTIGARRTGQ | |
| Dominant | PGWFLDSPDRPWNPPTFSPALLVVTEGDNATFTCSFSNTSESF | 8 |
| negative high | HVIWHRESPSGQTDTLAAFPEDRSQPGQDCRFRVTQLPNGRD | |
| affinity PD1 | FHMSVVRARRNDSGTYVCGVISLAPKIQIKESLRAELRVTER | |
| | RAEVPTAHPSPSPRPAGQFQTLVVGVVGGLLGSLVLLVWVL | |
| | AVICSRAARGTIGARRTGQ | |

### B. Transmembrane Domains

The PD-1 chimeric cytokine receptors of the disclosure comprise transmembrane domains. Such transmembrane domains are coupled to the extracellular PD-1 ectodomain on the N-terminus, and to additional intracellular/cytoplasmic domains on the C-terminus. In some embodiments, the coupling is achieved optionally through a linker. In some embodiments, the linker can comprise one or more amino acid residues.

As used herein, the transmembrane domains are capable of insertion into the membrane of a cell in which it is expressed. In some embodiments, the transmembrane domains of the disclosure span a cellular membrane, and comprise an extracellular portion, and/or an intracellular portion.

In some embodiments, the transmembrane domains of the disclosure are engineered and do not resemble any naturally occurring transmembrane domain, e.g. they are non-naturally occurring.

In other embodiments, the transmembrane domains of the disclosure are derived from naturally occurring receptors.

In some embodiments, the transmembrane and/or JAK domains of the disclosure are derived from, for example, one or more of the following receptors: erythropoietin receptor (EpoR), Interleukin 6 signal transducer (GP130 or IL6ST), prolactin receptor (PrlR), growth hormone receptor (GHR), granulocyte colony-stimulating factor receptor (GCSFR), and thrombopoietin receptor/myeloproliferative leukemia protein receptor (TPOR/MPLR). When derived from naturally occurring receptors, the entire receptor, or the entire transmembrane sequence of the receptor may not be necessary to effectuate constitutive activation and constitutive JAK binding/activation on the intracellular portion. Accordingly fragments of naturally occurring receptors may be utilized. Furthermore, certain mutations may be introduced into the transmembrane domains derived from naturally occurring receptors, to further tune the downstream JAK-dependent signaling.

In some embodiments, the transmembrane and/or JAK domains of the disclosure is derived from the naturally occurring EpoR receptor.

In some embodiments, the transmembrane and/or JAK domains of the disclosure is derived from the naturally occurring GP130 receptor.

In some embodiments, the transmembrane and/or JAK domains of the disclosure is derived from the naturally occurring PrlR receptor.

In some embodiments, the transmembrane and/or JAK domains of the disclosure is derived from the naturally occurring GHR receptor.

In some embodiments, the transmembrane and/or JAK domains of the disclosure is derived from the naturally occurring GCSF receptor.

In some embodiments, the transmembrane and/or JAK domains of the disclosure is derived from the naturally occurring TPOR receptor.

Table 3 provides exemplary full length sequences of naturally occurring receptors provided in the disclosure, from which the transmembrane and/or JAK domains are derived.

TABLE 3
Exemplary Naturally Occurring Receptors

| | SEQ ID |
|---|---|
| Naturally Occurring Receptor Name | NO: |
| >AAI12154.1 Erythropoietin receptor [Homo sapiens] | 9 |
| MDHLGASLWPQVGSLCLLLAGAAWAPPPNLPDPKFESKAALLAARGPEELLCFTERLEDLVCFWEEAASA | |
| GVGPGNYSFSYQLEDEPWKLCRLHQAPTARGAVRFWCSLPTADTSSFVPLELRVTAASGAPRYHRVIHIN | |
| EVVLLDAPVGLVARLADESGHVVLRWLPPPETPMTSHIRYEVDVSAGNGAGSVQRVEILEGRTECVLSNL | |
| RGRTRYTFAVRARMAEPSFGGFWSAWSEPVSLLTPSDLDPLILTLSLILVVILVLLTVLALLSHRRALKQ | |
| KIWPGIPSPESEFEGLFTTHKGNFQLWLYQNDGCLWWSPCTPFTEDPPASLEVLSERCWGTMQAVEPGTD | |
| DEGPLLEPVGSEHAQDTYLVLDKWLLPRNPPSEDLPGPGGSVDIVAMDEGSEASSCSSALASKPSPEGAS | |
| AASFEYTILDPSSQLLRPWTLCPELPPTPPHLKYLYLVVSDSGISTDYSSGDSQGAQGGLSDGPYSNPYE | |
| NSLIPAAEPLPPSYVACS | |
| >AAI17403.1 Interleukin 6 signal transducer (GP130, oncostatin M receptor) | 10 |
| [Homo sapiens] | |
| MLTLQTWLVQALFIFLTTESTGELLDPCGYISPESPVVQLHSNFTAVCVLKEKCMDYFHVNANYIVWKTN | |
| HFTIPKEQYTIINRTASSVTFTDIASLNIQLTCNILTFGQLEQNVYGITIISGLPPEKPKNLSCIVNEGK | |
| KMRCEWDRGRETHLETNFTLKSEWATHKFADCKAKRDTPTSCTVDYSTVYFVNIEVWVEAENALGKVTSD | |
| HINFDPVYKVKPNPPHNLSVINSEELSSILKLTWTNPSIKSVIILKYNIQYRTKDASTWSQIPPEDTAST | |
| RSSFTVQDLKPFTEYVFRIRCMKEDGKGYWSDWSEEASGITYEDRPSKAPSFWYKIDPSHTQGYRTVQLV | |
| WKTLPPFEANGKILDYEVTLTRWKSHLQNYTVNATKLTVNLTNDRYVATLTVRNLVGKSDAAVLTIPACD | |
| FQATHPVMDLKAFPKDNMLWVEWTTPRESVKKYILEWCVLSDKAPCITDWQQEDGTVHRTYLRGNLAESK | |
| CYLITVTPVYADGPGSPESIKAYLKQAPPSKGPTVRTKKVGKNEAVLEWDQLPVDVQNGFIRNYTIFYRT | |
| IIGNETAVNVDSSHTEYTLSSLTSDTLYMVRMAAYTDEGGKDGPEFTFTTPKFAQGEIEAIVVPVCLAFL | |
| LTTLLGVLFCFNKRDLIKKHIWPNVPDPSKSHIAQWSPHTPPRHNFNSKDQMYSDGNFTDVSVVEIEAND | |
| KKPFPEDLKSLDLFKKEKINTEGHSSGIGGSSCMSSSRPSISSSDENESSQNTSSTVQYSTVVHSGYRHQ | |
| VPSVQVFSRSESTQPLLDSEERPEDLQLVDHVDGGDGILPRQQYFKQNCSQHESSPDISHFERSKQVSSV | |
| NEEDFVRLKQQISDHISQSCGSGQMKMFQEVSAADAFGPGTEGQVERFETVGMEAATDEGMPKSYLPQTV | |
| RQGGYMPQ | |
| >XP_011512371.1 prolactin receptor isoform X2 [Homo sapiens] | 11 |
| MKENVASATVFTLLLFLNTCLLNGQLPPGKPEIFKCRSPNKETFTCWWRPGTDGGLPTNYSLTYHREGET | |
| LMHECPDYITGGPNSCHFGKQYTSMWRTYIMMVNATNQMGSSFSDELYVDVTYIVQPDPPLELAVEVKQP | |
| EDRKPYLWIKWSPPTLIDLKTGWFTLLYEIRLKPEKAAEWEIHFAGQQTEFKILSLHPGQKYLVQVRCKP | |
| DHGYWSAWSPATFIQIPSDFTMNDTTVWISVAVLSAVICLIIVWAVALKGYSMVTCIFPPVPGPKIKGFD | |
| AHLLEKGKSEELLSALGCQDFPPTSDYEDLLVEYLEVDDSEDQHLMSVHSKEHPSQGMKPTYLDPDTDSG | |
| RGSCDSPSLLSEKCEEPQANPSTFYDPEVIEKPENPETTHTWDPQCISMEGKIPYFHAGGSKCSTWPLPQ | |
| PSQHNPRSSYHNITDVCELAVGPAGAPATLLNEAGKDALKSSQTIKSREEGKATQQREVESFHSETDQDT | |
| PWLLPQEKTPFGSAKPLDYVEIHKVNKDGALSLLPKQRENSGKPKKPGTPENNKEYAKVSGVMDNNILVL | |
| VPDPHAKNVACFEESAKEAPPSLEQNQAEKALANFTATSSKCRLQLGGLDYLDPACFTHSFH | |
| >NP_000154.1 growth hormone receptor isoform 1 precursor [Homo sapiens] | 12 |
| MDLWQLLLTLALAGSSDAFSGSEATAAILSRAPWSLQSVNPGLKTNSSKEPKFTKCRSPERETFSCHWTD | |
| EVHHGTKNLGPIQLFYTRRNTQEWTQEWKECPDYVSAGENSCYFNSSFTSIWIPYCIKLTSNGGTVDEKC | |
| FSVDEIVQPDPPIALNWTLLNVSLTGIHADIQVRWEAPRNADIQKGWMVLEYELQYKEVNETKWKMMDPI | |
| LTTSVPVYSLKVDKEYEVRVRSKQRNSGNYGEFSEVLYVTLPQMSQFTCEEDFYFPWLLIIIFGIFGLTV | |
| MLFVFLFSKQQRIKMLILPPVPVPKIKGIDPDLLKEGKLEEVNTILAIHDSYKPEFHSDDSWVEFIELDI | |
| DEPDEKTEESDTDRLLSSDHEKSHSNLGVKDGDSGRTSCCEPDILETDFNANDIHEGTSEVAQPQRLKGE | |
| ADLLCLDQKNQNNSPYHDACPATQQPSVIQAEKNKPQPLPTEGAESTHQAAHIQLSNPSSLSNIDFYAQV | |
| SDITPAGSVVLSPGQKNKAGMSQCDMHPEMVSLCQENFLMDNAYFCEADAKKCIPVAPHIKVESHIQPSL | |
| NQEDIYITTESLTTAAGRPGTGEHVPGSEMPVPDYTSIHIVQSPQGLILNATALPLPDKEFLSSCGYVST | |
| DQLNKIMP | |
| >XP_016855859.1 granulocyte colony-stimulating factor receptor isoform X1 | 13 |
| [Homo sapiens] | |
| MARLGNCSLTWAALIILLLPGSLEECGHISVSAPIVHLGDPITASCIIKQNCSHLDPEPQILWRLGAELQ | |
| PGGRQQRLSDGTQESIITLPHLNHTQAFLSCCLNWGNSLQILDQVELRAGYPPAIPHNLSCLMNLTTSSL | |
| ICQWEPGPETHLPTSFTLKSFKSRGNCQTQGDSILDCVPKDGQSHCCIPRKHLLLYQNMGIWVQAENALG | |
| TSMSPQLCLDPMDVVKLEPPMLRTMDPSPEAAPPQAGCLQLCWEPWQPGLHINQKCELRHKPQRGEASWA | |
| LVGPLPLEALQYELCGLLPATAYTLQIRCIRWPLPGHWSDWSPSLELRTTERAPTVRLDTWWRQRQLDPR | |
| TVQLFWKPVPLEEDSGRIQGYVVSWRPSGQAGAILPLCNTTELSCTFHLPSEAQEVALVAYNSAGTSRPT | |
| PVVFSESRGPALTRLHAMARDPHSLWVGWEPPNPWPQGYVIEWGLGPPSASNSNKTWRMEQNGRATGFLL | |
| KENIRPFQLYEIIVTPLYQDTMGPSQHVYAYSQEMAPSHAPELHLKHIGKTWAQLEWVPEPPELGKSPLT | |
| HYTIFWTNAQNQSFSAILNASSRGFVLHGLEPASLYHIHLMAASQAGATNSTVLTLMTLTPEGSELHIIL | |
| GLFGLLLLLTCLCGTAWLCCSPNRKNPLWPSVPDPAHSSLGSWVPTIMEELPGPRQGQWLGQTSEMSRAL | |
| TPHPCVQDAFQLPGLGTPPITKLTVLEEDEKKPVPWESHNSSETCGLPTLVQTYVLQGDPRAVSTQPQSQ | |
| SGTSDQVLYGQLLGSPTSPGPGHYLRCDSTQPLLAGLTPSPKSYENLWFQASPLGTLVTPAPSQEDDCVF | |
| GPLLNFPLLQGIRVHGMEALGSF | |
| >NP_005364.1 thrombopoietin receptor precursor [Homo sapiens] | 131 |
| MPSWALFMVTSCLLLAPQNLAQVSSQDVSLLASDSEPLKCFSRTFEDLTCFWDEEEAAPSGTYQLLYAYP | |
| REKPRACPLSSQSMPHFGTRYVCQFPDQEEVRLFFPLHLWVKNVFLNQTRTQRVLFVDSVGLPAPPSIIK | |
| AMGGSQPGELQISWEEPAPEISDFLRYELRYGPRDPKNSTGPTVIQLIATETCCPALQRPHSASALDQSP | |
| CAQPTMPWQDGPKQTSPSREASALTAEGGSCLISGLQPGNSYWLQLRSEPDGISLGGSWGSWSLPVTVDL | |
| PGDAVALGLQCFTLDLKNVTCQWQQQDHASSQGFFYHSRARCCPRDRYPIWENCEEEEKTNPGLQTPQFS | |
| RCHFKSRNDSIIHILVEVTTAPGTVHSYLGSPFWIHQAVRLPTPNLHWREISSGHLELEWQHPSSWAAQE | |
| TCYQLRYTGEGHQDWKVLEPPLGARGGTLELRPRSRYRLQLRARLNGPTYQGPWSSWSDPTRVETATETA | |
| WISLVTALHLVLGLSAVLGLLLLRWQFPAHYRRLRHALWPSLPDLHRVLGQYLRDTAALSPPKATVSDTC | |
| EEVEPSLLEILPKSSERTPLPLCSSQAQMDYRRLQPSCLGTMPLSVCPPMAESGSCCTTHIANHSYLPLS | |
| YWQQP | |

In some embodiments, the transmembrane domain of the disclosure is derived from a truncated, or otherwise modified version of the naturally occurring TPOR/MPLR receptor shown in Table 3.

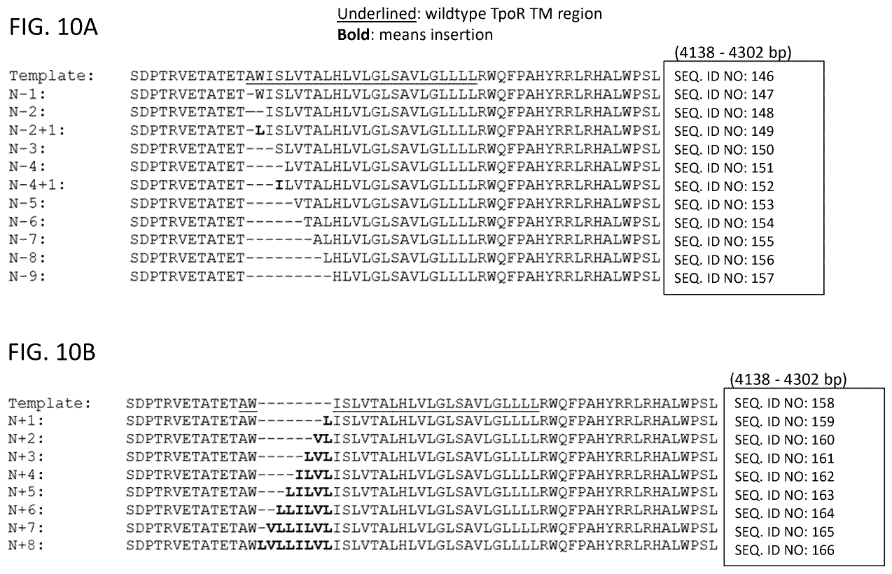

FIGS. 10A-B show the amino acid sequences for the wild type TpoR and the various transmembrane deletion (FIG. 10A) or insertion (FIG. 10B) variants.

Table 4 shows exemplary transmembrane amino acid sequences, coupled to intracellular JAK2 binding domain sequences.

In some embodiments, the transmembrane domain of the PD-1 chimeric cytokine receptor comprises the amino acid sequence of SEQ ID NO: 14. In some embodiments, the transmembrane domain of the PD-1 chimeric cytokine receptor comprises the amino acid sequence of SEQ ID NO: 15. In some embodiments, the transmembrane domain of the PD-1 chimeric cytokine receptor comprises the amino acid sequence of SEQ ID NO: 16. In some embodiments, the transmembrane domain of the PD-1 chimeric cytokine receptor comprises the amino acid sequence of SEQ ID NO: 17. In some embodiments, the transmembrane domain of the PD-1 chimeric cytokine receptor comprises the amino acid sequence of SEQ ID NO: 18. In some embodiments, the transmembrane domain of the PD-1 chimeric cytokine receptor comprises the amino acid sequence of SEQ ID NO: 19. In some embodiments, the transmembrane domain of the PD-1 chimeric cytokine receptor comprises the amino acid sequence of SEQ ID NO: 20. In some embodiments, the transmembrane domain of the PD-1 chimeric cytokine receptor comprises the amino acid sequence of SEQ ID NO: 21. In some embodiments, the transmembrane domain of the PD-1 chimeric cytokine receptor comprises the amino acid sequence of SEQ ID NO: 22. In some embodiments, the transmembrane domain of the PD-1 chimeric cytokine receptor comprises the amino acid sequence of SEQ ID NO: 23. In some embodiments, the transmembrane domain of the PD-1 chimeric cytokine receptor comprises the amino acid sequence of SEQ ID NO: 24. In some embodiments, the transmembrane domain of the PD-1 chimeric cytokine receptor comprises the amino acid sequence of SEQ ID NO: 25. In some embodiments, the transmembrane domain of the PD-1 chimeric cytokine receptor comprises the amino acid sequence of SEQ ID NO: 26. In some embodiments, the transmembrane domain of the PD-1 chimeric cytokine receptor comprises the amino acid sequence of SEQ ID NO: 27. In some embodiments, the transmembrane domain of the PD-1 chimeric cytokine receptor comprises the amino acid sequence of SEQ ID NO: 28. In some embodiments, the transmembrane domain of the PD-1 chimeric cytokine receptor comprises the amino acid sequence of SEQ ID NO: 29. In some embodiments, the transmembrane domain of the PD-1 chimeric cytokine receptor comprises the amino acid sequence of SEQ ID NO: 30. In some embodiments, the transmembrane domain of the PD-1 chimeric cytokine receptor comprises the amino acid sequence of SEQ ID NO: 31. In some embodiments, the transmembrane domain of the PD-1 chimeric cytokine receptor comprises the amino acid sequence of SEQ ID NO: 32. In some embodiments, the transmembrane domain of the PD-1 chimeric cytokine receptor comprises the amino acid sequence of SEQ ID NO: 33. In some embodiments, the transmembrane domain of the PD-1 chimeric cytokine receptor comprises the amino acid sequence of SEQ ID NO: 34. In some embodiments, the transmembrane domain of the PD-1 chimeric cytokine receptor comprises the amino acid sequence of SEQ ID NO: 35. In some embodiments, the transmembrane domain of the PD-1 chimeric cytokine receptor comprises the amino acid sequence of SEQ ID NO: 36. In some embodiments, the transmembrane domain of the PD-1 chimeric cytokine receptor comprises the amino acid sequence of SEQ ID NO: 37. In some embodiments, the transmembrane domain of the PD-1 chimeric cytokine receptor comprises the amino acid sequence of SEQ ID NO: 38. In some embodiments, the transmembrane domain of the PD-1 chimeric cytokine receptor comprises the amino acid sequence of SEQ ID NO: 39. In some embodiments, the transmembrane domain of the PD-1 chimeric cytokine receptor comprises the amino acid sequence of SEQ ID NO: 40. In some embodiments, the transmembrane domain of the PD-1 chimeric cytokine receptor comprises the amino acid sequence of SEQ ID NO: 41. In some embodiments, the transmembrane domain of the PD-1 chimeric cytokine receptor comprises the amino acid sequence of SEQ ID NO: 42. In some embodiments, the transmembrane domain of the PD-1 chimeric cytokine receptor comprises the amino acid sequence of SEQ ID NO: 43. In some embodiments, the transmembrane domain of the PD-1 chimeric cytokine receptor comprises the amino acid sequence of SEQ ID NO: 44 . . . . In some embodiments, the transmembrane domain of the PD-1 chimeric cytokine receptor comprises the amino acid sequence of SEQ ID NO: 134. In some embodiments, the transmembrane domain of the PD-1 chimeric cytokine receptor comprises the amino acid sequence of SEQ ID NO: 135. In some embodiments, the PD-1 chimeric cytokine receptor of the disclosure comprises an amino acid sequence of the transmembrane+JAK2 binding domain that is at least 80%, 85%, 90%, 95%, 98%, 99%, or 100% identical to the amino acid sequence of any one of the sequences shown in Table 4.

TABLE 4
Exemplary Transmembrane + JAK2 Binding Domain Sequences

| Transmembrane and | | SEQ |
|---|---|---|
| JAK2 binding domain | Amino acid sequence | ID NO: |
| GCSFR(614-710) | LTLMTLTPEGSELHIILGLFGLLLLLTCLCGTAWL | 14 |
| | CCSPNRKNPLWPSVPDPAHSSLGSWVPTIMEEDA | |
| | FQLPGLGTPPITKLTVLEEDEKKPVPWE | |
| GP130(609-700) | TTPKFAQGEIEAIVVPVCLAFLLTTLLGVLFCFNK | 15 |
| | RDLIKKHIWPNVPDPSKSHIAQWSPHTPPRHNFN | |
| | SKDQMYSDGNFTDVSVVEIEAND | |
| TPOR/MPLR(478-582) | SDPTRVETATETAWISLVTALHLVLGLSAVLGLL | 16 |
| | LLRWQFPAHYRRLRHALWPSLPDLHRVLGQYLR | |
| | DTAALSPPKATVSDTCEEVEPSLLEILPKSSERTPL | |
| | PL | |
| TPOR/MPLR(N-1) | SDPTRVETATETWISLVTALHLVLGLSAVLGLLL | 17 |
| | LRWQFPAHYRRLRHALWPSLPDLHRVLGQYLRD | |
| | TAALSPPKATVSDTCEEVEPSLLEILPKSSERTPLP | |
| | L | |
| TPOR/MPLR(N-2) | SDPTRVETATETISLVTALHLVLGLSAVLGLLLLR | 18 |
| | WQFPAHYRRLRHALWPSLPDLHRVLGQYLRDT | |
| | AALSPPKATVSDTCEEVEPSLLEILPKSSERTPLPL | |
| TPOR/MPLR(N-2 + 1) | SDPTRVETATETLISLVTALHLVLGLSAVLGLLLL | 19 |
| | RWQFPAHYRRLRHALWPSLPDLHRVLGQYLRDT | |
| | AALSPPKATVSDTCEEVEPSLLEILPKSSERTPLPL | |
| TPOR/MPLR(N-3) | SDPTRVETATETSLVTALHLVLGLSAVLGLLLLR | 20 |
| | WQFPAHYRRLRHALWPSLPDLHRVLGQYLRDT | |
| | AALSPPKATVSDTCEEVEPSLLEILPKSSERTPLPL | |
| TPOR/MPLR(N-4) | SDPTRVETATETLVTALHLVLGLSAVLGLLLLRW | 21 |
| | QFPAHYRRLRHALWPSLPDLHRVLGQYLRDTAA | |
| | LSPPKATVSDTCEEVEPSLLEILPKSSERTPLPL | |
| TPOR/MPLR(N-4 + 1) | SDPTRVETATETILVTALHLVLGLSAVLGLLLLR | 22 |
| | WQFPAHYRRLRHALWPSLPDLHRVLGQYLRDT | |
| | AALSPPKATVSDTCEEVEPSLLEILPKSSERTPLPL | |
| TPOR/MPLR(N-5) | SDPTRVETATETVTALHLVLGLSAVLGLLLLRW | 23 |
| | QFPAHYRRLRHALWPSLPDLHRVLGQYLRDTAA | |
| | LSPPKATVSDTCEEVEPSLLEILPKSSERTPLPL | |
| TPOR/MPLR(N-6) | SDPTRVETATETTALHLVLGLSAVLGLLLLRWQF | 24 |
| | PAHYRRLRHALWPSLPDLHRVLGQYLRDTAALS | |
| | PPKATVSDTCEEVEPSLLEILPKSSERTPLPL | |
| TPOR/MPLR(N-7) | SDPTRVETATETALHLVLGLSAVLGLLLLRWQFP | 25 |
| | AHYRRLRHALWPSLPDLHRVLGQYLRDTAALSP | |
| | PKATVSDTCEEVEPSLLEILPKSSERTPLPL | |
| TPOR/MPLR(N-8) | SDPTRVETATETLHLVLGLSAVLGLLLLRWQFPA | 26 |
| | HYRRLRHALWPSLPDLHRVLGQYLRDTAALSPP | |
| | KATVSDTCEEVEPSLLEILPKSSERTPLPL | |
| TPOR/MPLR(N-9) | SDPTRVETATETHLVLGLSAVLGLLLLRWQFPAH | 27 |
| | YRRLRHALWPSLPDLHRVLGQYLRDTAALSPPK | |
| | ATVSDTCEEVEPSLLEILPKSSERTPLPL | |
| TPOR/MPLR(N-10) | SDPTRVETATETLVLGLSAVLGLLLLRWQFPAHY | 28 |
| | RRLRHALWPSLPDLHRVLGQYLRDTAALSPPKA | |
| | TVSDTCEEVEPSLLEILPKSSERTPLPL | |
| TPOR/MPLR(N-11) | SDPTRVETATETVLGLSAVLGLLLLRWQFPAHYR | 29 |
| | RLRHALWPSLPDLHRVLGQYLRDTAALSPPKAT | |
| | VSDTCEEVEPSLLEILPKSSERTPLPL | |
| TPOR/MPLR(N-12) | SDPTRVETATETLGLSAVLGLLLLRWQFPAHYRR | 30 |
| | LRHALWPSLPDLHRVLGQYLRDTAALSPPKATV | |
| | SDTCEEVEPSLLEILPKSSERTPLPL | |
| TPOR/MPLR(N-13) | SDPTRVETATETGLSAVLGLLLLRWQFPAHYRRL | 31 |
| | RHALWPSLPDLHRVLGQYLRDTAALSPPKATVS | |
| | DTCEEVEPSLLEILPKSSERTPLPL | |
| TPOR/MPLR(N-14) | SDPTRVETATETLSAVLGLLLLRWQFPAHYRRLR | 32 |
| | HALWPSLPDLHRVLGQYLRDTAALSPPKATVSD | |
| | TCEEVEPSLLEILPKSSERTPLPL | |
| TPOR/MPLR(N-15) | SDPTRVETATETSAVLGLLLLRWQFPAHYRRLRH | 33 |
| | ALWPSLPDLHRVLGQYLRDTAALSPPKATVSDT | |
| | CEEVEPSLLEILPKSSERTPLPL | |
| TPOR/MPLR(N-16) | SDPTRVETATETAVLGLLLLRWQFPAHYRRLRH | 34 |
| | ALWPSLPDLHRVLGQYLRDTAALSPPKATVSDT | |
| | CEEVEPSLLEILPKSSERTPLPL | |
| TPOR/MPLR(N-17) | SDPTRVETATETVLGLLLLRWQFPAHYRRLRHA | 35 |
| | LWPSLPDLHRVLGQYLRDTAALSPPKATVSDTCE | |
| | EVEPSLLEILPKSSERTPLPL | |
| TPOR/MPLR(N-18) | SDPTRVETATETLGLLLLRWQFPAHYRRLRHAL | 36 |
| | WPSLPDLHRVLGQYLRDTAALSPPKATVSDTCEE | |
| | VEPSLLEILPKSSERTPLPL | |
| TPOR/MPLR(N + 1) | SDPTRVETATETAWLISLVTALHLVLGLSAVLGL | 37 |
| | LLLRWQFPAHYRRLRHALWPSLPDLHRVLGQYL | |
| | RDTAALSPPKATVSDTCEEVEPSLLEILPKSSERTP | |
| | LPL | |
| TPOR/MPLR(N + 2) | SDPTRVETATETAWVLISLVTALHLVLGLSAVLG | 38 |
| | LLLLRWQFPAHYRRLRHALWPSLPDLHRVLGQY | |
| | LRDTAALSPPKATVSDTCEEVEPSLLEILPKSSER | |
| | TPLPL | |
| TPOR/MPLR(N + 3) | SDPTRVETATETAWLVLISLVTALHLVLGLSAVL | 39 |
| | GLLLLRWQFPAHYRRLRHALWPSLPDLHRVLGQ | |
| | YLRDTAALSPPKATVSDTCEEVEPSLLEILPKSSE | |
| | RTPLPL | |
| TPOR/MPLR(N + 4) | SDPTRVETATETAWILVLISLVTALHLVLGLSAVL | 40 |
| | GLLLLRWQFPAHYRRLRHALWPSLPDLHRVLGQ | |
| | YLRDTAALSPPKATVSDTCEEVEPSLLEILPKSSE | |
| | RTPLPL | |
| TPOR/MPLR(N + 5) | SDPTRVETATETAWLILVLISLVTALHLVLGLSAV | 41 |
| | LGLLLLRWQFPAHYRRLRHALWPSLPDLHRVLG | |
| | QYLRDTAALSPPKATVSDTCEEVEPSLLEILPKSS | |
| | ERTPLPL | |
| TPOR/MPLR(N + 6) | SDPTRVETATETAWLLILVLISLVTALHLVLGLSA | 42 |
| | VLGLLLLRWQFPAHYRRLRHALWPSLPDLHRVL | |
| | GQYLRDTAALSPPKATVSDTCEEVEPSLLEILPKS | |
| | SERTPLPL | |
| TPOR/MPLR(N + 7) | SDPTRVETATETAWVLLILVLISLVTALHLVLGLS | 43 |
| | AVLGLLLLRWQFPAHYRRLRHALWPSLPDLHRV | |
| | LGQYLRDTAALSPPKATVSDTCEEVEPSLLEILPK | |
| | SSERTPLPL | |
| TPOR/MPLR(N + 8) | SDPTRVETATETAWLVLLILVLISLVTALHLVLGL | 44 |
| | SAVLGLLLLRWQFPAHYRRLRHALWPSLPDLHR | |
| | VLGQYLRDTAALSPPKATVSDTCEEVEPSLLEILP | |
| | KSSERTPLPL | |
| TPOR/MPLR(478-582; | SDPTRVETATETAWISLVTALLLVLGLNAVLGLL | 134 |
| H499L, S505N, W515K) | LLRKQFPAHYRRLRHALWPSLPDLHRVLGQYLR | |
| | DTAALSPPKATVSDTCEEVEPSLLEILPKSSERTPL | |
| | PL | |
| TPOR/MPLR(478-582; | SDPTRVETATETAWISLVTALHLVLGLNAVLGLL | 135 |
| S505N, W515K) | LLRKQFPAHYRRLRHALWPSLPDLHRVLGQYLR | |
| | DTAALSPPKATVSDTCEEVEPSLLEILPKSSERTPL | |
| | PL | |

### C. Janus Kinase (JAK)-Binding Domains

The PD-1 chimeric cytokine receptors of the disclosure comprise intracellular JAK-binding domains. The JAK-binding domain is coupled to the C-terminus of the transmembrane domain, either directly, or via a linker. In some embodiments, the linker can comprise one or more amino acid residues. The JAK-binding domain is coupled to the transmembrane domain on the intracellular side of the chimeric cytokine receptor.

In some embodiments, the JAK-binding domain is a JAK-1-binding domain, a JAK-2 binding domain, a JAK-3 binding domain, or a TYK2 binding domain.

In some embodiments, the JAK-binding domains of the PD-1 chimeric cytokine receptors of the disclosure are naturally occurring, and derived from a naturally occurring receptor.

In some embodiments, the JAK-binding domains of the PD-1 chimeric cytokine receptors of the disclosure are synthetic.

Table 4 provides exemplary amino acid sequences for Transmembrane and JAK2 Binding domains of the disclosure.

### D. Recruiting Domains

The PD-1 chimeric cytokine receptors of the disclosure comprise cytoplasmic domains comprising recruiting domains. The recruiting domain can be a STAT-recruiting domain, an AP1-recruiting domain, a Myc/Max-recruiting domain; or an NFkB-recruiting domain. In some embodiments, the recruiting domain is a Signal Transducer and Activator of Transcription (STAT)—recruiting (Stat-activating) domains from receptor tails (cytotails) or from cytokine receptor tails. These intracellular recruiting domains of the PD-1 chimeric cytokine receptors of the disclosure allow for the propagation of Signal 3 in an immune cell comprising a CAR and a chimeric cytokine receptor (e.g. a CAR-T-cell with a chimeric cytokine receptor of the disclosure). Cytokine signaling propagated through the Stat-recruiting domain allows for the cytokine-based immune potentiation of the cell. In some embodiments, the immune-potentiation is homeostatic, e.g. signaling gives rise to increase in immune cells bearing the CAR. In some embodiments, the immune-potentiation is inflammatory, e.g. signaling gives rise to increase in the potency of the immune cells bearing the CAR. In some embodiments, the immune-potentiation prevents exhaustion, e.g. signaling maintains the long-term functionality of immune cells bearing the CAR.

In some embodiments, the recruiting domains of the disclosure are synthetic, and do not resemble any naturally occurring receptor fragment.

In some embodiments, the Stat-recruiting domains of the disclosure are synthetic, and do not resemble any naturally occurring receptor fragment.

In some embodiments the recruiting domain is connected to the C-terminus of the JAK binding domain, either directly, or via a linker. In some embodiments, the linker can comprise one or more amino acid residues.

In other embodiments, the Stat-recruiting domains of the disclosure are derived from cytoplasmic tails of naturally occurring receptors, e.g. derived from naturally occurring cytokine receptors. These cytoplasmic tails of naturally occurring receptors may be the regions downstream of the JAK-activating domains of the transmembrane domain of the receptor. The Stat-recruiting domains of the chimeric cytokine receptors comprise at least one STAT-recruiting domain from at least one receptor. In some embodiments, the Stat-recruiting domain comprises at least one STAT1-recruiting domain. In some embodiments, the Stat-recruiting domain comprises at least one STAT2-recruiting domain. In some embodiments, the Stat-recruiting domain comprises at least one STAT3-recruiting domain. In some embodiments, the Stat-recruiting domain comprises at least one STAT4-recruiting domain. In some embodiments, the Stat-recruiting domain comprises at least one STAT5-recruiting domain. In some embodiments, the Stat-recruiting domain comprises at least one STAT6-recruiting domain. In some embodiments, the Stat-recruiting domain comprises at least one STAT7-recruiting domain.

In some embodiments, the naturally occurring receptor from which the Stat-recruiting domain is derived, is a not a cytokine receptor.

In some embodiments, the naturally occurring receptor from which the Stat-recruiting domain is derived, is a cytokine receptor. Exemplary cytokine receptors through which T-cell-immune potentiating cytokines signal include, but are not limited to IL-2 receptor, IL-7 receptor, IL-15 receptor and IL-21 receptor. In alternative embodiments, the receptor from which the Stat-recruiting domain is derived, is not a cytokine receptor. By choosing the Stat-recruiting domain of the chimeric cytokine receptor, the receptor can be redirected to signaling of choice.

Table 5 provides exemplary receptors from which Stat-recruiting domains of the chimeric cytokine receptors of the disclosure are derived. Table 6a provides exemplary amino acid sequences of recruiting domains of the disclosure.

In some embodiments, the PD-1 chimeric cytokine receptor of the disclosure comprises the amino acid sequence of the STAT-recruiting domain of SEQ ID NO: 45. In some embodiments, the PD-1 chimeric cytokine receptor of the disclosure comprises the amino acid sequence of the STAT-recruiting domain of SEQ ID NO: 46. In some embodiments, the PD-1 chimeric cytokine receptor of the disclosure comprises the amino acid sequence of the STAT-recruiting domain of SEQ ID NO: 47. In some embodiments, the PD-1 chimeric cytokine receptor of the disclosure comprises the amino acid sequence of the STAT-recruiting domain of SEQ ID NO: 48. In some embodiments, the PD-1 chimeric cytokine receptor of the disclosure comprises the amino acid sequence of the STAT-recruiting domain of SEQ ID NO: 49. In some embodiments, the PD-1 chimeric cytokine receptor of the disclosure comprises the amino acid sequence of the STAT-recruiting domain of SEQ ID NO: 50. In some embodiments, the PD-1 chimeric cytokine receptor of the disclosure comprises the amino acid sequence of the STAT-recruiting domain of SEQ ID NO: 51. In some embodiments, the PD-1 chimeric cytokine receptor of the disclosure comprises the amino acid sequence of the STAT-recruiting domain of SEQ ID NO: 52. In some embodiments, the PD-1 chimeric cytokine receptor of the disclosure comprises the amino acid sequence of the STAT-recruiting domain of SEQ ID NO: 53. In some embodiments, the PD-1 chimeric cytokine receptor of the disclosure comprises the amino acid sequence of the STAT-recruiting domain of SEQ ID NO: 54. In some embodiments, the PD-1 chimeric cytokine receptor of the disclosure comprises the amino acid sequence of the STAT-recruiting domain of SEQ ID NO: 55. In some embodiments, the PD-1 chimeric cytokine receptor of the disclosure comprises the amino acid sequence of the STAT-recruiting domain of SEQ ID NO: 56. In some embodiments, the PD-1 chimeric cytokine receptor of the disclosure comprises the amino acid sequence of the STAT-recruiting domain of SEQ ID NO: 57. In some embodiments, the PD-1 chimeric cytokine receptor of the disclosure comprises the amino acid sequence of the STAT-recruiting domain of SEQ ID NO: 58. In some embodiments, the PD-1 chimeric cytokine receptor of the disclosure comprises the amino acid sequence of the STAT-recruiting domain of SEQ ID NO: 59. In some embodiments, the PD-1 chimeric cytokine receptor of the disclosure comprises the amino acid sequence of the STAT-recruiting domain of SEQ ID NO: 60. In some embodiments, the PD-1 chimeric cytokine receptor of the disclosure comprises the amino acid sequence of the STAT-recruiting domain of SEQ ID NO: 61. In some embodiments, the PD-1 chimeric cytokine receptor of the disclosure comprises the amino acid sequence of the STAT-recruiting domain of SEQ ID NO: 62. In some embodiments, the PD-1 chimeric cytokine receptor of the disclosure comprises the amino acid sequence of the STAT-recruiting domain of SEQ ID NO: 63. In some embodiments, the PD-1 chimeric cytokine receptor of the disclosure comprises the amino acid sequence of the STAT-recruiting domain of SEQ ID NO: 64. In some embodiments, the PD-1 chimeric cytokine receptor of the disclosure comprises the amino acid sequence of the STAT-recruiting domain of SEQ ID NO: 65. In some embodiments, the PD-1 chimeric cytokine receptor of the disclosure comprises the amino acid sequence of the STAT-recruiting domain of SEQ ID NO: 66. In some embodiments, the PD-1 chimeric cytokine receptor of the disclosure comprises the amino acid sequence of the STAT-recruiting domain of SEQ ID NO: 67. In some embodiments, the PD-1 chimeric cytokine receptor of the disclosure comprises the amino acid sequence of the STAT-recruiting domain of SEQ ID NO: 68. In some embodiments, the PD-1 chimeric cytokine receptor of the disclosure comprises the amino acid sequence of the STAT-recruiting domain of SEQ ID NO: 69. In some embodiments, the PD-1 chimeric cytokine receptor of the disclosure comprises the amino acid sequence of the STAT-recruiting domain of SEQ ID NO: 70. In some embodiments, the PD-1 chimeric cytokine receptor of the disclosure comprises the amino acid sequence of the STAT-recruiting domain of SEQ ID NO: 71. In some embodiments, the PD-1 chimeric cytokine receptor of the disclosure comprises the amino acid sequence of the STAT-recruiting domain of SEQ ID NO: 72. In some embodiments, the PD-1 chimeric cytokine receptor of the disclosure comprises the amino acid sequence of the STAT-recruiting domain of SEQ ID NO: 73. In some embodiments, the PD-1 chimeric cytokine receptor of the disclosure comprises the amino acid sequence of the STAT-recruiting domain of SEQ ID NO: 74. In some embodiments, the PD-1 chimeric cytokine receptor of the disclosure comprises the amino acid sequence of the STAT-recruiting domain of SEQ ID NO: 75. In some embodiments, the PD-1 chimeric cytokine receptor of the disclosure comprises the amino acid sequence of the STAT-recruiting domain of SEQ ID NO: 76. In some embodiments, the PD-1 chimeric cytokine receptor of the disclosure comprises the amino acid sequence of the STAT-recruiting domain of SEQ ID NO: 77. In some embodiments, the PD-1 chimeric cytokine receptor of the disclosure comprises the amino acid sequence of the STAT-recruiting domain of SEQ ID NO: 78. In some embodiments, the PD-1 chimeric cytokine receptor of the disclosure comprises the amino acid sequence of the STAT-recruiting domain of SEQ ID NO: 79. In some embodiments, the PD-1 chimeric cytokine receptor of the disclosure comprises the amino acid sequence of the STAT-recruiting domain of SEQ ID NO: 80. In some embodiments, the PD-1 chimeric cytokine receptor of the disclosure comprises the amino acid sequence of the STAT-recruiting domain of SEQ ID NO: 81. In some embodiments, the PD-1 chimeric cytokine receptor of the disclosure comprises the amino acid sequence of the STAT-recruiting domain of SEQ ID NO: 82. In some embodiments, the PD-1 chimeric cytokine receptor of the disclosure comprises the amino acid sequence of the STAT-recruiting domain of SEQ ID NO: 83. In some embodiments, the PD-1 chimeric cytokine receptor of the disclosure comprises the amino acid sequence of the STAT-recruiting domain of SEQ ID NO: 84. In some embodiments, the PD-1 chimeric cytokine receptor of the disclosure comprises the amino acid sequence of the STAT-recruiting domain of SEQ ID NO: 85. In some embodiments, the PD-1 chimeric cytokine receptor of the disclosure comprises the amino acid sequence of the STAT-recruiting domain of SEQ ID NO: 86. In some embodiments, the PD-1 chimeric cytokine receptor of the disclosure comprises the amino acid sequence of the STAT-recruiting domain of SEQ ID NO: 87. In some embodiments, the PD-1 chimeric cytokine receptor of the disclosure comprises the amino acid sequence of the STAT-recruiting domain of SEQ ID NO: 170. In some embodiments, the PD-1 chimeric cytokine receptor of the disclosure comprises the amino acid sequence of the STAT-recruiting domain of SEQ ID NO: 171. In some embodiments, the PD-1 chimeric cytokine receptor of the disclosure comprises an amino acid sequence of the STAT-recruiting domain that is at least 80%, 85%, 90%, 95%, 98%, 99%, or 100% identical to the amino acid sequence of any one of the sequences shown in Table 6a.

TABLE 5
Source for
recruiting
domains
BLNK
IL2RG
EGFR
EpoR
GHR
IFNAR1
IFNAR2
IFNAR1/2
IFNLR1
IL10R1
IL12Rb1
IL12Rb2
IL21R
IL2Rb
IL2small
IL7R
IL7Ra
IL9R
IL15R
IL21R

TABLE 6a
Recruiting domain Sequences (Cytotail Sequences)

| | | SEQ |
|---|---|---|
| Recruiting domain | Amino acid sequence | ID NO: |
| IL7R(316-459) | ARDEVEGFLQDTFPQQLEESEKQRLGGDVQSPN | 45 |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| IL2Rb(333-551) | VTQLLLQQDKVPEPASLSSNHSLTSCFTNQGYFF | 46 |
| | FHLPDALEIEACQVYFTYDPYSEEDPDEGVAGAP | |
| | TGSSPQPLQPLSGEDDAYCTFPSRDDLLLFSPSLL | |
| | GGPSPPSTAPGGSGAGEERMPPSLQERVPRDWDP | |
| | QPLGPPTPGVPDLVDFQPPPELVLREAGEEVPDA | |
| | GPREGVSFPWSRPPGQGEFRALNARLPLNTDAYL | |
| | SLQELQGQDPTHLV | |
| IFNAR1(508-557) | ISTIATVEETNQTDEDHKKYSSQTSQDSGNYSNE | 47 |
| | DESESKTSEELQQDFV | |
| IFNAR2(310-515) | KKKVWDYNYDDESDSDTEAAPRTSGGGYTMHG | 48 |
| | LTVRPLGQASATSTESQLIDPESEEEPDLPEVDVE | |
| | LPTMPKDSPQQLELLSGPCERRKSPLQDPFPEEDY | |
| | SSTEGSGGRITFNVDLNSVFLRVLDDEDSDDLEA | |
| | PLMLSSHLEEMVDPEDPDNVQSNHLLASGEGTQ | |
| | PTFPSPSSEGLWSEDAPSDQSDTSESDVDLGDGYI | |
| | MR | |
| IFNAR1/2(IFNAR1 | ISTIATVEETNQTDEDHKKYSSQTSQDSGNYSNE | 49 |
| residues 508-557-IFNAR2 | DESESKTSEELQQDFVKKKVWDYNYDDESDSDT | |
| residues 310-515) | EAAPRTSGGGYTMHGLTVRPLGQASATSTESQLI | |
| | DPESEEEPDLPEVDVELPTMPKDSPQQLELLSGPC | |
| | ERRKSPLQDPFPEEDYSSTEGSGGRITFNVDLNSV | |
| | FLRVLDDEDSDDLEAPLMLSSHLEEMVDPEDPD | |
| | NVQSNHLLASGEGTQPTFPSPSSEGLWSEDAPSD | |
| | QSDTSESDVDLGDGYIMR | |
| IFNLR1(300-520) | RGVRPTPRVRAPATQQTRWKKDLAEDEEEEDEE | 50 |
| | DTEDGVSFQPYIEPPSFLGQEHQAPGHSEAGGVD | |
| | SGRPRAPLVPSEGSSAWDSSDRSWASTVDSSWD | |
| | RAGSSGYLAEKGPGQGPGGDGHQESLPPPEFSKD | |
| | SGFLEELPEDNLSSWATWGTLPPEPNLVPGGPPV | |
| | SLQTLTFCWESSPEEEEEARESEIEDSDAGSWGAE | |
| | STQRTEDRGRTLGHYMAR | |
| Common Gamma | IPPKGGALGEGPGASPCNQHSPYWAPPCYTLKPE | 51 |
| Chain(335-369) | T | |
| IL9R(356-521) | TALLTCGPARPWKSVALEEEQEGPGTRLPGNLSS | 52 |
| | EDVLPAGCTEWRVQTLAYLPQEDWAPTSLTRPA | |
| | PPDSEGSRSSSSSSSSNNNNYCALGCYGGWHLSA | |
| | LPGNTQSSGPIPALACGLSCDHQGLETQQGVAW | |
| | VLAGHCQRPGLHEDLQGMLLPSVLSKARSWTF | |
| IL21R(322-538) | PRSPAKRLQLTELQEPAELVESDGVPKPSFWPTA | 53 |
| | QNSGGSAYSEERDRPYGLVSIDTVTVLDAEGPCT | |
| | WPCSCEDDGYPALDLDAGLEPSPGLEDPLLDAG | |
| | TTVLSCGCVSAGSPGLGGPLGSLLDRLKPPLADG | |
| | EDWAGGLPWGGRSPGGVSESEAGSPLAGLDMD | |
| | TFDSGFVGSDCSSPVECDFTSPGDEGPPRSYLRQ | |
| | WVVIPPPLSSPGPQAS | |
| GHR(353-638) | PDEKTEESDTDRLLSSDHEKSHSNLGVKDGDSGR | 54 |
| | TSCCEPDILETDFNANDIHEGTSEVAQPQRLKGE | |
| | ADLLCLDQKNQNNSPYHDACPATQQPSVIQAEK | |
| | NKPQPLPTEGAESTHQAAHIQLSNPSSLSNIDFYA | |
| | QVSDITPAGSVVLSPGQKNKAGMSQCDMHPEM | |
| | VSLCQENFLMDNAYFCEADAKKCIPVAPHIKVES | |
| | HIQPSLNQEDIYITTESLTTAAGRPGTGEHVPGSE | |
| | MPVPDYTSIHIVQSPQGLILNATALPLPDKEFLSS | |
| | CGYVSTDQLNKIMP | |
| EpoR(339-508) | WGTMQAVEPGTDDEGPLLEPVGSEHAQDTYLVL | 55 |
| | DKWLLPRNPPSEDLPGPGGSVDIVAMDEGSEASS | |
| | CSSALASKPSPEGASAASFEYTILDPSSQLLRPWT | |
| | LCPELPPTPPHLKYLYLVVSDSGISTDYSSGDSQG | |
| | AQGGLSDGPYSNPYENSLIPAAEPLPPSYVACS | |
| murine IL2Rb(337-539) | AVQLLLLQKDSAPLPSPSGHSQASCFTNQGYFFF | 56 |
| | HLPNALEIESCQVYFTYDPCVEEEVEEDGSRLPE | |
| | GSPHPPLLPLAGEQDDYCAFPPRDDLLLFSPSLST | |
| | PNTAYGGSRAPEERSPLSLHEGLPSLASRDLMGL | |
| | QRPLERMPEGDGEGLSANSSGEQASVPEGNLHG | |
| | QDQDRGQGPILTLNTDAYLSLQELQAQDSVHLI | |
| murine IL7Ra(316-459) | ARDEVESFLPNDLPAQPEELETQGHRAAVHSAN | 57 |
| | RSPETSVSPPETVRRESPLRCLARNLSTCNAPPLL | |
| | SSRSPDYRDGDRNRPPVYQDLLPNSGNTNVPVPV | |
| | PQPLPFQSGILIPVSQRQPISTSSVLNQEEAYVTMS | |
| | SFYQNK | |
| EGFR(955-1186) | VIQGDERMHLPSPTDSNFYRALMDEEDMDDVVD | 58 |
| | ADEYLIPQQGFFSSPSTSRTPLLSSLSATSNNSTVA | |
| | CIDRNGLQSCPIKEDSFLQRYSSDPTGALTEDSID | |
| | DTFLPVPEYINQSVPKRPAGSVQNPVYHNQPLNP | |
| | APSRDPHYQDPHSTAVGNPEYLNTVQPTCVNSTF | |
| | DSPAHWAQKGSHQISLDNPDYQQDFFPKEAKPN | |
| | GIFKGSTAENAEYLRVAPQSSEFIGA | |
| EGFR(955-1186; | VIQGDERMHLPSPTDSNFFRALMDEEDMDDVVD | 59 |
| Y974F, d1045-1057) | ADEYLIPQQGFFSSPSTSRTPLLSSLSATSNNSTVA | |
| | CIDRNGLQSCPIKEDSFLQRIDDTFLPVPEYINQSV | |
| | PKRPAGSVQNPVYHNQPLNPAPSRDPHYQDPHS | |
| | TAVGNPEYLNTVQPTCVNSTFDSPAHWAQKGSH | |
| | QISLDNPDYQQDFFPKEAKPNGIFKGSTAENAEY | |
| | LRVAPQSSEFIGA | |
| EGFR(955-1009; Y974F) | VIQGDERMHLPSPTDSNFFRALMDEEDMDDVVD | 60 |
| | ADEYLIPQQGFFSSPSTSRTP | |
| EGFR(1019-1085) | NNSTVACIDRNGLQSCPIKEDSFLQRIDDTFLPVP | 61 |
| | EYINQSVPKRPAGSVQNPV | |
| EGFR(1037-1103; | KEDSFLQRIDDTFLPVPEFINQSVPKRPAGSVQNP | 62 |
| Y1068/1101F, d1045-1057) | VYHNQPLNPAPSRDPHFQD | |
| EGFR(1066-1118; | VPEFINQSVPKRPAGSVQNPVFHNQPLNPAPSRD | 63 |
| Y1068/1086F) | PHYQDPHSTAVGNPEYLNTV | |
| EGFR(1122-1165) | PEYLNTVQPTCVNSTFDSPAHWAQKGSHQISLDN | 64 |
| | PDYQQDFFPKEAKPNGIFKG | |
| EGFR(1133-1186; | WAQKGSHQISLDNPDFQQDFFPKEAKPNGIFKGS | 65 |
| Y1148F) | TAENAEYLRVAPQSSEFIGA | |
| IL12Rb2(775-825) | SDPKPENPACPWTVLPAGDLPTHDGYLPSNIDDL | 66 |
| | PSHEAPLADSLEELEPQ | |
| IL7R(376-416) | ACDAPILSSSRSLDCRESGKNGPHVYQDLLLSLG | 67 |
| | TTNSTLP | |
| IL7R(424-459) | GILTLNPVAQGQPILTSLGSNQEEAYVTMSSFYQ | 68 |
| | NQ | |
| IL7R(376-416, 424-459) | ACDAPILSSSRSLDCRESGKNGPHVYQDLLLSLG | 69 |
| | TTNSTLPQGQPILTSLGSNQEEAYVTMSSFYQNQ | |
| IL7R(424-459; Y456F) | GILTLNPVAQGQPILTSLGSNQEEAYVTMSSFFQN | 70 |
| | Q | |
| IL7R(376-416, 424-459, | ACDAPILSSSRSLDCRESGKNGPHVYQDLLLSLG | 71 |
| Y456F) | TTNSTLPQGQPILTSLGSNQEEAYVTMSSFFQNQ | |
| IL2Rbsmall(393-433) | DEGVAGAPTGSSPQPLQPLSGEDDAYCTFPSRDD | 72 |
| | LLLFSPSGQGEFRALNARLPLNTDAYLSLQELQG | |
| | QDPTHLV | |
| IL2Rbsmall(518-551) | GQGEFRALNARLPLNTDAYLSLQELQGQDPTHL | 73 |
| | V | |
| IL2Rbsmall(339-379, 393- | QQDKVPEPASLSSNHSLTSCFTNQGYFFFHLPDA | 74 |
| 433) | LEIEACQDEGVAGAPTGSSPQPLQPLSGEDDAYC | |
| | TFPSRDDLLLFSPS | |
| IL2Rbsmall(339-379, 518- | QQDKVPEPASLSSNHSLTSCFTNQGYFFFHLPDA | 75 |
| 551) | LEIEACQ | |
| | GQGEFRALNARLPLNTDAYLSLQELQGQDPTHL | |
| | V | |
| IL2Rbsmall(393-433, 518- | DEGVAGAPTGSSPQPLQPLSGEDDAYCTFPSRDD | 76 |
| 551) | LLLFSPSGQGEFRALNARLPLNTDAYLSLQELQG | |
| | QDPTHLV | |
| IL2Rbsmall(339-379, 393- | QQDKVPEPASLSSNHSLTSCFTNQGYFFFHLPDA | 77 |
| 433, 518-551) | LEIEACQDEGVAGAPTGSSPQPLQPLSGEDDAYC | |
| | TFPSRDDLLLFSPSGQGEFRALNARLPLNTDAYLS | |
| | LQELQGQDPTHLV | |
| IFNAR2small(310-352) | KKKVWDYNYDDESDSDTEAAPRTSGGGYTMHG | 78 |
| | LTVRPLGQASA | |
| IFNAR2small(486-515) | EGLWSEDAPSDQSDTSESDVDLGDGYIMR | 79 |
| IFNAR2small(310-352, | KKKVWDYNYDDESDSDTEAAPRTSGGGYTMHG | 80 |
| 486-515) | LTVRPLGQASA | |
| | EGLWSEDAPSDQSDTSESDVDLGDGYIMR | |
| BLNK(53-208) | ASESPADEEEQWSDDFDSDYENPDEHSDSEMYV | 81 |
| | MPAEENADDSYEPPPVEQETRPVHPALPFARGEY | |
| | IDNRSSQRHSPPFSKTLPSKPSWPSEKARLTSTLP | |
| | ALTALQKPQVPPKPKGLLEDEADYVVPVEDNDE | |
| | NYIHPTESSSPPPEKAPMVNR | |
| BLNK(53-208; Y72F) | ASESPADEEEQWSDDFDSDFENPDEHSDSEMYV | 82 |
| | MPAEENADDSYEPPPVEQETRPVHPALPFARGEY | |
| | IDNRSSQRHSPPFSKTLPSKPSWPSEKARLTSTLP | |
| | ALTALQKPQVPPKPKGLLEDEADYVVPVEDNDE | |
| | NYIHPTESSSPPPEKAPMVNR | |
| BLNK(53-208; | ASESPADEEEQWSDDFDSDFENPDEHSDSEMYV | 83 |
| Y72F, Y96F) | MPAEENADDSFEPPPVEQETRPVHPALPFARGEY | |
| | IDNRSSQRHSPPFSKTLPSKPSWPSEKARLTSTLP | |
| | ALTALQKPQVPPKPKGLLEDEADYVVPVEDNDE | |
| | NYIHPTESSSPPPEKAPMVNR | |
| EpoR(339-508) | WGTMQAVEPGTDDEGPLLEPVGSEHAQDTYLVL | 84 |
| | DKWLLPRNPPSEDLPGPGGSVDIVAMDEGSEASS | |
| | CSSALASKPSPEGASAASFEYTILDPSSQLLRPWT | |
| | LCPELPPTPPHLKYLYLVVSDSGISTDYSSGDSQG | |
| | AQGGLSDGPYSNPYENSLIPAAEPLPPSYVACS | |
| IL12Rb2(714-862) | VTPVFRHPPCSNWPQREKGIQGHQASEKDMMHS | 85 |
| | ASSPPPPRALQAESRQLVDLYKVLESRGSDPKPE | |
| | NPACPWTVLPAGDLPTHDGYLPSNIDDLPSHEAP | |
| | LADSLEELEPQHISLSVFPSSSLHPLTFSCGDKLTL | |
| | DQLKMRCDSLML | |
| IL12Rb1(622-662) | WDKGERTEPLEKTELPEGAPELALDTELSLEDGD | 86 |
| | RCKAKM | |
| IL10R1(304-578) | VSPELKNLDLHGSTDSGFGSTKPSLQTEEPQFLLP | 87 |
| | DPHPQADRTLGNREPPVLGDSCSSGSSNSTDSGIC | |
| | LQEPSLSPSTGPTWEQQVGSNSRGQDDSGIDLVQ | |
| | NSEGRAGDTQGGSALGHHSPPEPEVPGEEDPAA | |
| | VAFQGYLRQTRCAEEKATKTGCLEEESPLTDGL | |
| | GPKFGRCLVDEAGLHPPALAKGYLKQDPLEMTL | |
| | ASSGAPTGQWNQPTEEWSLLALSSCSDLGISDWS | |
| | FAHDLAPLGCVAAPGGLLGSFNSDLVTLPLISSLQ | |
| | SSE | |
| IL2Rb(333-551, | VTQLLLQQDKVPEPASLSSNHSLTSCFTNQGYFF | 170 |
| Y381S, Y384S, Y387S) | FHLPDALEIEACQVSFTSDPSSEEDPDEGVAGAPT | |
| | GSSPQPLQPLSGEDDAYCTFPSRDDLLLFSPSLLG | |
| | GPSPPSTAPGGSGAGEERMPPSLQERVPRDWDPQ | |
| | PLGPPTPGVPDLVDFQPPPELVLREAGEEVPDAG | |
| | PREGVSFPWSRPPGQGEFRALNARLPLNTDAYLS | |
| | LQELQGQDPTHLV | |
| IL2Rb(333-551, | VTQLLLQQDKVPEPASLSSNHSLTSCFTNQGSFFF | 171 |
| Y364S, Y381S, Y384S, | HLPDALEIEACQVSFTSDPSSEEDPDEGVAGAPTG | |
| Y387S) | SSPQPLQPLSGEDDAYCTFPSRDDLLLFSPSLLGG | |
| | PSPPSTAPGGSGAGEERMPPSLQERVPRDWDPQP | |
| | LGPPTPGVPDLVDFQPPPELVLREAGEEVPDAGP | |
| | REGVSFPWSRPPGQGEFRALNARLPLNTDAYLSL | |
| | QELQGQDPTHLV | |

In some embodiments, the Stat-recruiting domain of a chimeric cytokine receptor of the disclosure comprises a STAT-recruiting domain from one receptor.

In order to generate multiple outputs, one or more STAT-recruiting domains may be joined in tandem to mimic signaling from one or more cytokines.

In some embodiments, the STAT-recruiting domain comprises portions of more than one receptor, e.g. comprising more than one STAT-recruiting domain. In such embodiments, a tandem cytokine signaling domain is provided, allowing for enhanced signaling. Accordingly, in some embodiments, the STAT-recruiting domain of a monomer of the chimeric cytokine receptor of the disclosure comprises the STAT-recruiting domains from more than one receptor, e.g. comprises the STAT-recruiting domains from two, three, four, five, or even six receptors. For example, in some embodiments, STAT-recruiting domains can be linked in tandem to stimulate multiple pathways (e.g., the IL7R(316-459)-IL12Rb2(775-825) fragment fusion for pro-persistence STAT5 and pro-inflammatory STAT4; IL7R(316-459)-IL2Rbsmall(393-433,518-551) for pro-persistence; IL7R(316-459)-EGFR(1122-1165) for pro-persistence and anti-exhaustion; IL2Rbsmall(393-433,518-551)-EGFR(1122-1165) for pro-persistence and anti-exhaustion).

In some embodiments, the more than one STAT-recruiting domains can be connected either directly, or via a linker. In some embodiments, the linker can comprise one or more amino acid residues.

When generating multiple outputs, the proximity of individual STAT-recruiting domains to the cell membrane can influence the strength of their respective signaling outputs. Table 6b shows examples of PD-1 chimeric cytokine receptors with the dual outputs, where each output can be placed either proximal or distal to the cell membrane.

TABLE 6b
Examples of PD-1 chimeric cytokine receptors with dual outputs

| Dual output STAT-recruiting | Membrane | Membrane |
|---|---|---|
| domain | proximal | distal |
| IL2Rbsmall(393-433, | IL2Rbsmall | IL21R(322-538) |
| 518-551)/IL21R(322-538) | (393-433, | |
| | 518-551) | |
| IL21R(322-538)/ | IL21R(322-538) | IL2Rbsmall |
| IL2Rbsmall(393-433, 518-551) | | (393-433, 518-551) |
| IL2Rbsmall(339-379, | IL2Rbsmall | IL21R(322-538) |
| 393-433, 518-551)/ | (339-379, 393-433, | |
| IL21R(322-538) | 518-551) | |
| IL21R(322-538)/ | IL21R(322-538) | IL2Rbsmall |
| IL2Rbsmall(339-379, | | (339-379, 393-433, |
| 393-433, 518-551) | | 518-551) |
| IL7R (316-459)/IL21R(322-538) | IL7R (316-459) | IL21R(322-538) |
| IL21R(322-538)/IL7R (316-459) | IL21R(322-538) | IL7R (316-459) |

Without being bound to theory or mechanism, in some embodiments, a JAK-protein (JAK1, JAK2, JAK3, or TYK2) is bound to a PD-1 chimeric cytokine receptor of the disclosure (comprising a PD-1 ectodomain, a transmembrane domain, a JAK-binding domain, and a recruiting domain). In some embodiments, in the presence of (e.g. binding to) PD-L1, PD-L2, or an anti-PD-1 antibody, the PD-1 chimeric cytokine receptor clusters and allows for the two bound JAK-proteins to become activated, which in turn phosphorylate tyrosine residues on the recruiting domain of the chimeric receptor. The phosphorylated recruiting domains are then capable of binding the recruited proteins (e.g. a phosphorylated STAT-recruiting domain binds a STAT-protein), which in turn effectuate transcription events in the nucleus.

### E. Exemplary PD-1 Chimeric Cytokine Receptors

Table 7 shows exemplary context-dependent cytokine receptor sequences of the disclosure. The receptors may be expressed with a signal sequence, e.g. a CD8SS of SEQ ID NO: 1.

In some embodiments, the PD-1 chimeric cytokine receptor of the disclosure comprises the amino acid sequence of SEQ ID NO: 88. In some embodiments, the PD-1 chimeric cytokine receptor of the disclosure comprises the amino acid sequence of SEQ ID NO: 89. In some embodiments, the PD-1 chimeric cytokine receptor of the disclosure comprises the amino acid sequence of SEQ ID NO: 90. In some embodiments, the PD-1 chimeric cytokine receptor of the disclosure comprises the amino acid sequence of SEQ ID NO: 91. In some embodiments, the PD-1 chimeric cytokine receptor of the disclosure comprises the amino acid sequence of SEQ ID NO: 92. In some embodiments, the PD-1 chimeric cytokine receptor of the disclosure comprises the amino acid sequence of SEQ ID NO: 93. In some embodiments, the PD-1 chimeric cytokine receptor of the disclosure comprises the amino acid sequence of SEQ ID NO: 94. In some embodiments, the PD-1 chimeric cytokine receptor of the disclosure comprises the amino acid sequence of SEQ ID NO: 95. In some embodiments, the PD-1 chimeric cytokine receptor of the disclosure comprises the amino acid sequence of SEQ ID NO: 96. In some embodiments, the PD-1 chimeric cytokine receptor of the disclosure comprises the amino acid sequence of SEQ ID NO: 97. In some embodiments, the PD-1 chimeric cytokine receptor of the disclosure comprises the amino acid sequence of SEQ ID NO: 98. In some embodiments, the PD-1 chimeric cytokine receptor of the disclosure comprises the amino acid sequence of SEQ ID NO: 99. In some embodiments, the PD-1 chimeric cytokine receptor of the disclosure comprises the amino acid sequence of SEQ ID NO: 100. In some embodiments, the PD-1 chimeric cytokine receptor of the disclosure comprises the amino acid sequence of SEQ ID NO: 101. In some embodiments, the PD-1 chimeric cytokine receptor of the disclosure comprises the amino acid sequence of SEQ ID NO: 102. In some embodiments, the PD-1 chimeric cytokine receptor of the disclosure comprises the amino acid sequence of SEQ ID NO: 103. In some embodiments, the PD-1 chimeric cytokine receptor of the disclosure comprises the amino acid sequence of SEQ ID NO: 104. In some embodiments, the PD-1 chimeric cytokine receptor of the disclosure comprises the amino acid sequence of SEQ ID NO: 105. In some embodiments, the PD-1 chimeric cytokine receptor of the disclosure comprises the amino acid sequence of SEQ ID NO: 106. In some embodiments, the PD-1 chimeric cytokine receptor of the disclosure comprises the amino acid sequence of SEQ ID NO: 107. In some embodiments, the PD-1 chimeric cytokine receptor of the disclosure comprises the amino acid sequence of SEQ ID NO: 108. In some embodiments, the PD-1 chimeric cytokine receptor of the disclosure comprises the amino acid sequence of SEQ ID NO: 109. In some embodiments, the PD-1 chimeric cytokine receptor of the disclosure comprises the amino acid sequence of SEQ ID NO: 110. In some embodiments, the PD-1 chimeric cytokine receptor of the disclosure comprises the amino acid sequence of SEQ ID NO: 111. In some embodiments, the PD-1 chimeric cytokine receptor of the disclosure comprises the amino acid sequence of SEQ ID NO: 112. In some embodiments, the PD-1 chimeric cytokine receptor of the disclosure comprises the amino acid sequence of SEQ ID NO: 113. In some embodiments, the PD-1 chimeric cytokine receptor of the disclosure comprises the amino acid sequence of SEQ ID NO: 114. In some embodiments, the PD-1 chimeric cytokine receptor of the disclosure comprises the amino acid sequence of SEQ ID NO: 115. In some embodiments, the PD-1 chimeric cytokine receptor of the disclosure comprises the amino acid sequence of SEQ ID NO: 116. In some embodiments, the PD-1 chimeric cytokine receptor of the disclosure comprises the amino acid sequence of SEQ ID NO: 117. In some embodiments, the PD-1 chimeric cytokine receptor of the disclosure comprises the amino acid sequence of SEQ ID NO: 118. In some embodiments, the PD-1 chimeric cytokine receptor of the disclosure comprises the amino acid sequence of SEQ ID NO: 119. In some embodiments, the PD-1 chimeric cytokine receptor of the disclosure comprises the amino acid sequence of SEQ ID NO: 120. In some embodiments, the PD-1 chimeric cytokine receptor of the disclosure comprises the amino acid sequence of SEQ ID NO: 121. In some embodiments, the PD-1 chimeric cytokine receptor of the disclosure comprises the amino acid sequence of any one of SEQ ID NOs: 88-121, SEQ ID NOs: 136-145, and SEQ ID NOs: 172-213. In some embodiments, the PD-1 chimeric cytokine receptor of the disclosure comprises an amino acid sequence that is at least 80%, 85%, 90%, 95%, 98%, 99%, or 100% identical to the amino acid sequence of any one of SEQ ID NOs: 88-121, SEQ ID NOs: 172-213.

TABLE 7
Exemplary PD1 chimeric cytokine receptor sequences (assembled PD1 chimeric
cytokine receptors):

| PD1 | | |
|---|---|---|
| chimeric | | |
| cytokine | | SEQ |
| receptor | Amino acid sequence | ID NO: |
| WTPD1(21-207). | PGWFLDSPDRPWNPPTFSPALLVVTEGDNATFTCSFSNTSESF | 88 |
| TpoR(478-582). | VLNWYRMSPSNQTDKLAAFPEDRSQPGQDCRFRVTQLPNGR | |
| IL7Ra(316-459). | DFHMSVVRARRNDSGTYLCGAISLAPKAQIKESLRAELRVTE | |
| IL12Rb2 | RRAEVPTAHPSPSPRPAGQFQTLVVGVVGGLLGSLVLLVWV | |
| (775-825) | LAVICSRAARGTIGARRTGQ | |
| | SDPTRVETATETAWISLVTALHLVLGLSAVLGLLLLRWQFPA | |
| | HYRRLRHALWPSLPDLHRVLGQYLRDTAALSPPKATVSDTC | |
| | EEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| WTPD1(21-207). | PGWFLDSPDRPWNPPTFSPALLVVTEGDNATFTCSFSNTSESF | 89 |
| GCSFR(614-710). | VLNWYRMSPSNQTDKLAAFPEDRSQPGQDCRFRVTQLPNGR | |
| IL7Ra(316-459). | DFHMSVVRARRNDSGTYLCGAISLAPKAQIKESLRAELRVTE | |
| IL12Rb2 | RRAEVPTAHPSPSPRPAGQFQTLVVGVVGGLLGSLVLLVWV | |
| (775-825) | LAVICSRAARGTIGARRTGQ | |
| | LTLMTLTPEGSELHIILGLFGLLLLLTCLCGTAWLCCSPNRKN | |
| | PLWPSVPDPAHSSLGSWVPTIMEEDAFQLPGLGTPPITKLTVL | |
| | EEDEKKPVPWE | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| WTPD1(21-207). | PGWFLDSPDRPWNPPTFSPALLVVTEGDNATFTCSFSNTSESF | 90 |
| GP130(609-700). | VLNWYRMSPSNQTDKLAAFPEDRSQPGQDCRFRVTQLPNGR | |
| IL7Ra(316-459). | DFHMSVVRARRNDSGTYLCGAISLAPKAQIKESLRAELRVTE | |
| IL12Rb2 | RRAEVPTAHPSPSPRPAGQFQTLVVGVVGGLLGSLVLLVWV | |
| (775-825) | LAVICSRAARGTIGARRTGQ | |
| | TTPKFAQGEIEAIVVPVCLAFLLTTLLGVLFCENKRDLIKKHI | |
| | WPNVPDPSKSHIAQWSPHTPPRHNFNSKDQMYSDGNFTDVS | |
| | VVEIEAND | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| 2xWTPD1(21-207). | PGWFLDSPDRPWNPPTFSPALLVVTEGDNATFTCSFSNTSESF | 91 |
| TpoR(478-582). | VLNWYRMSPSNQTDKLAAFPEDRSQPGQDCRFRVTQLPNGR | |
| IL7Ra(316-459). | DFHMSVVRARRNDSGTYLCGAISLAPKAQIKESLRAELRVTE | |
| IL12Rb2 | RRAEVPTAHPSPSPRPAGQFQTLVVGVVGGLLGSLVLLVWV | |
| (775-825) | LAVICSRAARGTIGARRTGQPGWFLDSPDRPWNPPTFSPALL | |
| | VVTEGDNATFTCSFSNTSESFVLNWYRMSPSNQTDKLAAFPE | |
| | DRSQPGQDCRFRVTQLPNGRDFHMSVVRARRNDSGTYLCG | |
| | AISLAPKAQIKESLRAELRVTERRAEVPTAHPSPSPRPAGQFQ | |
| | TLVVGVVGGLLGSLVLLVWVLAVICSRAARGTIGARRTGQ | |
| | SDPTRVETATETAWISLVTALHLVLGLSAVLGLLLLRWQFPA | |
| | HYRRLRHALWPSLPDLHRVLGQYLRDTAALSPPKATVSDTC | |
| | EEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| 3xWTPD1(21-207). | PGWFLDSPDRPWNPPTFSPALLVVTEGDNATFTCSFSNTSESF | 92 |
| TpoR(478-582). | VLNWYRMSPSNQTDKLAAFPEDRSQPGQDCRFRVTQLPNGR | |
| IL7Ra(316-459). | DFHMSVVRARRNDSGTYLCGAISLAPKAQIKESLRAELRVTE | |
| IL12Rb2 | RRAEVPTAHPSPSPRPAGQFQTLVVGVVGGLLGSLVLLVWV | |
| (775-825) | LAVICSRAARGTIGARRTGQPGWFLDSPDRPWNPPTFSPALL | |
| | VVTEGDNATFTCSFSNTSESFVLNWYRMSPSNQTDKLAAFPE | |
| | DRSQPGQDCRFRVTQLPNGRDFHMSVVRARRNDSGTYLCG | |
| | AISLAPKAQIKESLRAELRVTERRAEVPTAHPSPSPRPAGQFQ | |
| | TLVVGVVGGLLGSLVLLVWVLAVICSRAARGTIGARRTGQP | |
| | GWFLDSPDRPWNPPTFSPALLVVTEGDNATFTCSFSNTSESF | |
| | VLNWYRMSPSNQTDKLAAFPEDRSQPGQDCRFRVTQLPNGR | |
| | DFHMSVVRARRNDSGTYLCGAISLAPKAQIKESLRAELRVTE | |
| | RRAEVPTAHPSPSPRPAGQFQTLVVGVVGGLLGSLVLLVWV | |
| | LAVICSRAARGTIGARRTGQ | |
| | SDPTRVETATETAWISLVTALHLVLGLSAVLGLLLLRWQFPA | |
| | HYRRLRHALWPSLPDLHRVLGQYLRDTAALSPPKATVSDTC | |
| | EEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 93 |
| (478-582). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETAWISLVTALHLVLGLSAVLGLLLLRWQFPA | |
| | HYRRLRHALWPSLPDLHRVLGQYLRDTAALSPPKATVSDTC | |
| | EEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR(N-1). | PGWFLDSPDRPWNPPTFSPALLVVTEGDNATFTCSFSNTSESF | 94 |
| IL7Ra(316-459). | HVIWHRESPSGQTDTLAAFPEDRSQPGQDCRFRVTQLPNGRD | |
| IL12Rb2 | FHMSVVRARRNDSGTYVCGVISLAPKIQIKESLRAELRVTER | |
| (775-825) | RAEVPTAHPSPSPRPAGQFQTLVVGVVGGLLGSLVLLVWVL | |
| | AVICSRAARGTIGARRTGQ | |
| | SDPTRVETATETWISLVTALHLVLGLSAVLGLLLLRWQFPAH | |
| | YRRLRHALWPSLPDLHRVLGQYLRDTAALSPPKATVSDTCE | |
| | EVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR(N-2). | PGWFLDSPDRPWNPPTFSPALLVVTEGDNATFTCSFSNTSESF | 95 |
| IL7Ra(316-459). | HVIWHRESPSGQTDTLAAFPEDRSQPGQDCRFRVTQLPNGRD | |
| IL12Rb2 | FHMSVVRARRNDSGTYVCGVISLAPKIQIKESLRAELRVTER | |
| (775-825) | RAEVPTAHPSPSPRPAGQFQTLVVGVVGGLLGSLVLLVWVL | |
| | AVICSRAARGTIGARRTGQ | |
| | SDPTRVETATETISLVTALHLVLGLSAVLGLLLLRWQFPAHY | |
| | RRLRHALWPSLPDLHRVLGQYLRDTAALSPPKATVSDTCEE | |
| | VEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 96 |
| (N-2 + 1). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETLISLVTALHLVLGLSAVLGLLLLRWQFPAH | |
| | YRRLRHALWPSLPDLHRVLGQYLRDTAALSPPKATVSDTCE | |
| | EVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 97 |
| (N-3). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETSLVTALHLVLGLSAVLGLLLLRWQFPAHYR | |
| | RLRHALWPSLPDLHRVLGQYLRDTAALSPPKATVSDTCEEV | |
| | EPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 98 |
| (N-4). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETLVTALHLVLGLSAVLGLLLLRWQFPAHYR | |
| | RLRHALWPSLPDLHRVLGQYLRDTAALSPPKATVSDTCEEV | |
| | EPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 99 |
| (N-4 + 1). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETILVTALHLVLGLSAVLGLLLLRWQFPAHYR | |
| | RLRHALWPSLPDLHRVLGQYLRDTAALSPPKATVSDTCEEV | |
| | EPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 100 |
| (N-5). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETVTALHLVLGLSAVLGLLLLRWQFPAHYRR | |
| | LRHALWPSLPDLHRVLGQYLRDTAALSPPKATVSDTCEEVEP | |
| | SLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 101 |
| (N-6). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETTALHLVLGLSAVLGLLLLRWQFPAHYRRLR | |
| | HALWPSLPDLHRVLGQYLRDTAALSPPKATVSDTCEEVEPSL | |
| | LEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 102 |
| (N-7). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETALHLVLGLSAVLGLLLLRWQFPAHYRRLR | |
| | HALWPSLPDLHRVLGQYLRDTAALSPPKATVSDTCEEVEPSL | |
| | LEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 103 |
| (N-8). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETLHLVLGLSAVLGLLLLRWQFPAHYRRLRH | |
| | ALWPSLPDLHRVLGQYLRDTAALSPPKATVSDTCEEVEPSLL | |
| | EILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 104 |
| (N-9). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETHLVLGLSAVLGLLLLRWQFPAHYRRLRHA | |
| | LWPSLPDLHRVLGQYLRDTAALSPPKATVSDTCEEVEPSLLEI | |
| | LPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 105 |
| (N-10). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETLVLGLSAVLGLLLLRWQFPAHYRRLRHAL | |
| | WPSLPDLHRVLGQYLRDTAALSPPKATVSDTCEEVEPSLLEIL | |
| | PKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 106 |
| (N-11). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETVLGLSAVLGLLLLRWQFPAHYRRLRHALW | |
| | PSLPDLHRVLGQYLRDTAALSPPKATVSDTCEEVEPSLLEILP | |
| | KSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 107 |
| (N-12). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETLGLSAVLGLLLLRWQFPAHYRRLRHALWP | |
| | SLPDLHRVLGQYLRDTAALSPPKATVSDTCEEVEPSLLEILPK | |
| | SSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 108 |
| (N-13). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETGLSAVLGLLLLRWQFPAHYRRLRHALWPS | |
| | LPDLHRVLGQYLRDTAALSPPKATVSDTCEEVEPSLLEILPKS | |
| | SERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 109 |
| (N-14). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETLSAVLGLLLLRWQFPAHYRRLRHALWPSLP | |
| | DLHRVLGQYLRDTAALSPPKATVSDTCEEVEPSLLEILPKSSE | |
| | RTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 110 |
| (N-15). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETSAVLGLLLLRWQFPAHYRRLRHALWPSLP | |
| | DLHRVLGQYLRDTAALSPPKATVSDTCEEVEPSLLEILPKSSE | |
| | RTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 111 |
| (N-16). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETAVLGLLLLRWQFPAHYRRLRHALWPSLPD | |
| | LHRVLGQYLRDTAALSPPKATVSDTCEEVEPSLLEILPKSSER | |
| | TPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 112 |
| (N-17). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETVLGLLLLRWQFPAHYRRLRHALWPSLPDL | |
| | HRVLGQYLRDTAALSPPKATVSDTCEEVEPSLLEILPKSSERT | |
| | PLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 113 |
| (N-18). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETLGLLLLRWQFPAHYRRLRHALWPSLPDLH | |
| | RVLGQYLRDTAALSPPKATVSDTCEEVEPSLLEILPKSSERTP | |
| | LPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 114 |
| (N + 1). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETAWLISLVTALHLVLGLSAVLGLLLLRWQFP | |
| | AHYRRLRHALWPSLPDLHRVLGQYLRDTAALSPPKATVSDT | |
| | CEEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 115 |
| (N + 2). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETAWVLISLVTALHLVLGLSAVLGLLLLRWQF | |
| | PAHYRRLRHALWPSLPDLHRVLGQYLRDTAALSPPKATVSD | |
| | TCEEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.Tpo | PGWFLDSPDRPWNPPTFSPALLVVTEGDNATFTCSFSNTSESF | 116 |
| R(N + 3). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETAWLVLISLVTALHLVLGLSAVLGLLLLRWQ | |
| | FPAHYRRLRHALWPSLPDLHRVLGQYLRDTAALSPPKATVS | |
| | DTCEEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 117 |
| (N + 4). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETAWILVLISLVTALHLVLGLSAVLGLLLLRW | |
| | QFPAHYRRLRHALWPSLPDLHRVLGQYLRDTAALSPPKATV | |
| | SDTCEEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 118 |
| (N + 5). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETAWLILVLISLVTALHLVLGLSAVLGLLLLR | |
| | WQFPAHYRRLRHALWPSLPDLHRVLGQYLRDTAALSPPKAT | |
| | VSDTCEEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 119 |
| (N + 6). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETAWLLILVLISLVTALHLVLGLSAVLGLLLLR | |
| | WQFPAHYRRLRHALWPSLPDLHRVLGQYLRDTAALSPPKAT | |
| | VSDTCEEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 120 |
| (N + 7). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETAWVLLILVLISLVTALHLVLGLSAVLGLLLL | |
| | RWQFPAHYRRLRHALWPSLPDLHRVLGQYLRDTAALSPPKA | |
| | TVSDTCEEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1.TpoR | PGWFLDSPDRPWNPPTFSPALLVVTEGDNATFTCSFSNTSESF | 121 |
| (N + 8). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVVGVVGGLLGSLVLLVWVL | |
| (775-825) | AVICSRAARGTIGARRTGQ | |
| | SDPTRVETATETAWLVLLILVLISLVTALHLVLGLSAVLGLLL | |
| | LRWQFPAHYRRLRHALWPSLPDLHRVLGQYLRDTAALSPPK | |
| | ATVSDTCEEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| | SDPKPENPACPWTVLPAGDLPTHDGYLPSNIDDLPSHEAPLA | |
| | DSLEELEPQ | |
| HAPD1(21-170). | PGWFLDSPDRPWNPPTFSPALLVVTEGDNATFTCSFSNTSESF | 136 |
| TPOR/MPLR(478- | HVIWHRESPSGQTDTLAAFPEDRSQPGQDCRFRVTQLPNGRD | |
| 582; H499L, | FHMSVVRARRNDSGTYVCGVISLAPKIQIKESLRAELRVTER | |
| S505N, W515K). | RAEVPTAHPSPSPRPAGQFQTLV | |
| IL7Ra(316-459) | SDPTRVETATETAWISLVTALLLVLGLNAVLGLLLLRKQFPA | |
| | HYRRLRHALWPSLPDLHRVLGQYLRDTAALSPPKATVSDTC | |
| | EEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| HAPD1(21-170). | PGWFLDSPDRPWNPPTFSPALLVVTEGDNATFTCSFSNTSESF | 137 |
| TpoR(478-582; | HVIWHRESPSGQTDTLAAFPEDRSQPGQDCRFRVTQLPNGRD | |
| S505N, W515K). | FHMSVVRARRNDSGTYVCGVISLAPKIQIKESLRAELRVTER | |
| IL2small(393- | RAEVPTAHPSPSPRPAGQFQTLV | |
| 433, 518-551) | SDPTRVETATETAWISLVTALHLVLGLNAVLGLLLLRKQFPA | |
| | HYRRLRHALWPSLPDLHRVLGQYLRDTAALSPPKATVSDTC | |
| | EEVEPSLLEILPKSSERTPLPL | |
| | DEGVAGAPTGSSPQPLQPLSGEDDAYCTFPSRDDLLLFSPSG | |
| | QGEFRALNARLPLNTDAYLSLQELQGQDPTHLV | |
| HAPDIDN | PGWFLDSPDRPWNPPTFSPALLVVTEGDNATFTCSFSNTSESF | 138 |
| (HAPD1 | HVIWHRESPSGQTDTLAAFPEDRSQPGQDCRFRVTQLPNGRD | |
| Dominant | FHMSVVRARRNDSGTYVCGVISLAPKIQIKESLRAELRVTER | |
| Negative) | RAEVPTAHPSPSPRPAGQFQTLVVGVVGGLLGSLVLLVWVL | |
| | AVICSRAARGTIGARRTGQ | |
| HAPD1(21-170). | PGWFLDSPDRPWNPPTFSPALLVVTEGDNATFTCSFSNTSESF | 139 |
| TpoR(478-582). | HVIWHRESPSGQTDTLAAFPEDRSQPGQDCRFRVTQLPNGRD | |
| IL7Ra(316-459) | FHMSVVRARRNDSGTYVCGVISLAPKIQIKESLRAELRVTER | |
| | RAEVPTAHPSPSPRPAGQFQTLV | |
| | SDPTRVETATETAWISLVTALHLVLGLSAVLGLLLLRWQFPA | |
| | HYRRLRHALWPSLPDLHRVLGQYLRDTAALSPPKATVSDTC | |
| | EEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| HAPD1(21-170). | PGWFLDSPDRPWNPPTFSPALLVVTEGDNATFTCSFSNTSESF | 140 |
| TpoR(478-582). | HVIWHRESPSGQTDTLAAFPEDRSQPGQDCRFRVTQLPNGRD | |
| IL2small | FHMSVVRARRNDSGTYVCGVISLAPKIQIKESLRAELRVTER | |
| (393- | RAEVPTAHPSPSPRPAGQFQTLV | |
| 433, 518-551) | SDPTRVETATETAWISLVTALHLVLGLSAVLGLLLLRWQFPA | |
| | HYRRLRHALWPSLPDLHRVLGQYLRDTAALSPPKATVSDTC | |
| | EEVEPSLLEILPKSSERTPLPL | |
| | DEGVAGAPTGSSPQPLQPLSGEDDAYCTFPSRDDLLLFSPSG | |
| | QGEFRALNARLPLNTDAYLSLQELQGQDPTHLV | |
| WTPD1(21-170). | PGWFLDSPDRPWNPPTFSPALLVVTEGDNATFTCSFSNTSESF | 172 |
| TpoR(478-582). | VLNWYRMSPSNQTDKLAAFPEDRSQPGQDCRFRVTQLPNGR | |
| IL7Ra(316-459). | DFHMSVVRARRNDSGTYLCGAISLAPKAQIKESLRAELRVTE | |
| IL12Rb2 | RRAEVPTAHPSPSPRPAGQFQTLVSDPTRVETATETAWISLVT | |
| (775-825) | ALHLVLGLSAVLGLLLLRWQFPAHYRRLRHALWPSLPDLHR | |
| | VLGQYLRDTAALSPPKATVSDTCEEVEPSLLEILPKSSERTPL | |
| | PLLEARDEVEGFLQDTFPQQLEESEKQRLGGDVQSPNCPSED | |
| | VVITPESFGRDSSLTCLAGNVSACDAPILSSSRSLDCRESGKN | |
| | GPHVYQDLLLSLGTTNSTLPPPFSLQSGILTLNPVAQGQPILTS | |
| | LGSNQEEAYVTMSSFYQNQGSGSRSDPKPENPACPWTVLPA | |
| | GDLPTHDGYLPSNIDDLPSHEAPLADSLEELEPQ | |
| WTPD1(21-170). | PGWFLDSPDRPWNPPTFSPALLVVTEGDNATFTCSFSNTSESF | 173 |
| GCSFR(614-710). | VLNWYRMSPSNQTDKLAAFPEDRSQPGQDCRFRVTQLPNGR | |
| IL7Ra(316-459). | DFHMSVVRARRNDSGTYLCGAISLAPKAQIKESLRAELRVTE | |
| IL12Rb2 | RRAEVPTAHPSPSPRPAGQFQTLVLTLMTLTPEGSELHIILGLF | |
| (775-825) | GLLLLLTCLCGTAWLCCSPNRKNPLWPSVPDPAHSSLGSWV | |
| | PTIMEEDAFQLPGLGTPPITKLTVLEEDEKKPVPWELEARDEV | |
| | EGFLQDTFPQQLEESEKQRLGGDVQSPNCPSEDVVITPESFGR | |
| | DSSLTCLAGNVSACDAPILSSSRSLDCRESGKNGPHVYQDLL | |
| | LSLGTTNSTLPPPFSLQSGILTLNPVAQGQPILTSLGSNQEEAY | |
| | VTMSSFYQNQGSGSRSDPKPENPACPWTVLPAGDLPTHDGY | |
| | LPSNIDDLPSHEAPLADSLEELEPQ | |
| WTPD1(21-170). | PGWFLDSPDRPWNPPTFSPALLVVTEGDNATFTCSFSNTSESF | 174 |
| GP130(609-700). | VLNWYRMSPSNQTDKLAAFPEDRSQPGQDCRFRVTQLPNGR | |
| IL7Ra(316-459). | DFHMSVVRARRNDSGTYLCGAISLAPKAQIKESLRAELRVTE | |
| IL12Rb2 | RRAEVPTAHPSPSPRPAGQFQTLVTTPKFAQGEIEAIVVPVCL | |
| (775-825) | AFLLTTLLGVLFCFNKRDLIKKHIWPNVPDPSKSHIAQWSPHT | |
| | PPRHNFNSKDQMYSDGNFTDVSVVEIEANDLEARDEVEGFL | |
| | QDTFPQQLEESEKQRLGGDVQSPNCPSEDVVITPESFGRDSSL | |
| | TCLAGNVSACDAPILSSSRSLDCRESGKNGPHVYQDLLLSLG | |
| | TTNSTLPPPFSLQSGILTLNPVAQGQPILTSLGSNQEEAYVTM | |
| | SSFYQNQGSGSRSDPKPENPACPWTVLPAGDLPTHDGYLPSN | |
| | IDDLPSHEAPLADSLEELEPQ | |
| 2xWTPD1(21-170). | PGWFLDSPDRPWNPPTFSPALLVVTEGDNATFTCSFSNTSESF | 175 |
| TpoR(478-582). | VLNWYRMSPSNQTDKLAAFPEDRSQPGQDCRFRVTQLPNGR | |
| IL7Ra(316-459). | DFHMSVVRARRNDSGTYLCGAISLAPKAQIKESLRAELRVTE | |
| IL12Rb2 | RRAEVPTAHPSPSPRPAGQFQTLVGPGWFLDSPDRPWNPPTF | |
| (775-825) | SPALLVVTEGDNATFTCSFSNTSESFVLNWYRMSPSNQTDKL | |
| | AAFPEDRSQPGQDCRFRVTQLPNGRDFHMSVVRARRNDSGT | |
| | YLCGAISLAPKAQIKESLRAELRVTERRAEVPTAHPSPSPRPA | |
| | GQFQTLVSDPTRVETATETAWISLVTALHLVLGLSAVLGLLL | |
| | LRWQFPAHYRRLRHALWPSLPDLHRVLGQYLRDTAALSPPK | |
| | ATVSDTCEEVEPSLLEILPKSSERTPLPLLEARDEVEGFLQDTF | |
| | PQQLEESEKQRLGGDVQSPNCPSEDVVITPESFGRDSSLTCLA | |
| | GNVSACDAPILSSSRSLDCRESGKNGPHVYQDLLLSLGTTNS | |
| | TLPPPFSLQSGILTLNPVAQGQPILTSLGSNQEEAYVTMSSFY | |
| | QNQGSGSRSDPKPENPACPWTVLPAGDLPTHDGYLPSNIDDL | |
| | PSHEAPLADSLEELEPQ | |
| 3xWTPD1(21-170). | PGWFLDSPDRPWNPPTFSPALLVVTEGDNATFTCSFSNTSESF | 176 |
| TpoR(478-582). | VLNWYRMSPSNQTDKLAAFPEDRSQPGQDCRFRVTQLPNGR | |
| IL7Ra(316-459). | DFHMSVVRARRNDSGTYLCGAISLAPKAQIKESLRAELRVTE | |
| IL12Rb2 | RRAEVPTAHPSPSPRPAGQFQTLVGPGWFLDSPDRPWNPPTF | |
| (775-825) | SPALLVVTEGDNATFTCSFSNTSESFVLNWYRMSPSNQTDKL | |
| | AAFPEDRSQPGQDCRFRVTQLPNGRDFHMSVVRARRNDSGT | |
| | YLCGAISLAPKAQIKESLRAELRVTERRAEVPTAHPSPSPRPA | |
| | GQFQTLVGPGWFLDSPDRPWNPPTFSPALLVVTEGDNATFTC | |
| | SFSNTSESFVLNWYRMSPSNQTDKLAAFPEDRSQPGQDCRFR | |
| | VTQLPNGRDFHMSVVRARRNDSGTYLCGAISLAPKAQIKESL | |
| | RAELRVTERRAEVPTAHPSPSPRPAGQFQTLVSDPTRVETAT | |
| | ETAWISLVTALHLVLGLSAVLGLLLLRWQFPAHYRRLRHAL | |
| | WPSLPDLHRVLGQYLRDTAALSPPKATVSDTCEEVEPSLLEIL | |
| | PKSSERTPLPLLEARDEVEGFLQDTFPQQLEESEKQRLGGDV | |
| | QSPNCPSEDVVITPESFGRDSSLTCLAGNVSACDAPILSSSRSL | |
| | DCRESGKNGPHVYQDLLLSLGTTNSTLPPPFSLQSGILTLNPV | |
| | AQGQPILTSLGSNQEEAYVTMSSFYQNQGSGSRSDPKPENPA | |
| | CPWTVLPAGDLPTHDGYLPSNIDDLPSHEAPLADSLEELEPQ | |
| HAPD1(21-170). | PGWFLDSPDRPWNPPTFSPALLVVTEGDNATFTCSFSNTSESF | 177 |
| TpoR(478-582). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETAWISLVTA | |
| (775-825) | LHLVLGLSAVLGLLLLRWQFPAHYRRLRHALWPSLPDLHRV | |
| | LGQYLRDTAALSPPKATVSDTCEEVEPSLLEILPKSSERTPLPL | |
| | LEARDEVEGFLQDTFPQQLEESEKQRLGGDVQSPNCPSEDVV | |
| | ITPESFGRDSSLTCLAGNVSACDAPILSSSRSLDCRESGKNGP | |
| | HVYQDLLLSLGTTNSTLPPPFSLQSGILTLNPVAQGQPILTSLG | |
| | SNQEEAYVTMSSFYQNQGSGSRSDPKPENPACPWTVLPAGD | |
| | LPTHDGYLPSNIDDLPSHEAPLADSLEELEPQ | |
| HAPD1(21-170). | PGWFLDSPDRPWNPPTFSPALLVVTEGDNATFTCSFSNTSESF | 178 |
| TpoR(N-1). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETWISLVTAL | |
| (775-825) | HLVLGLSAVLGLLLLRWQFPAHYRRLRHALWPSLPDLHRVL | |
| | GQYLRDTAALSPPKATVSDTCEEVEPSLLEILPKSSERTPLPLL | |
| | EARDEVEGFLQDTFPQQLEESEKQRLGGDVQSPNCPSEDVVI | |
| | TPESFGRDSSLTCLAGNVSACDAPILSSSRSLDCRESGKNGPH | |
| | VYQDLLLSLGTTNSTLPPPFSLQSGILTLNPVAQGQPILTSLGS | |
| | NQEEAYVTMSSFYQNQGSGSRSDPKPENPACPWTVLPAGDL | |
| | PTHDGYLPSNIDDLPSHEAPLADSLEELEPQ | |
| HAPD1(21-170). | PGWFLDSPDRPWNPPTFSPALLVVTEGDNATFTCSFSNTSESF | 179 |
| TpoR(N-2). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETISLVTALH | |
| (775-825) | LVLGLSAVLGLLLLRWQFPAHYRRLRHALWPSLPDLHRVLG | |
| | QYLRDTAALSPPKATVSDTCEEVEPSLLEILPKSSERTPLPLLE | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQGSGSRSDPKPENPACPWTVLPAGDLP | |
| | THDGYLPSNIDDLPSHEAPLADSLEELEPQ | |
| HAPD1(21-170). | PGWFLDSPDRPWNPPTFSPALLVVTEGDNATFTCSFSNTSESF | 180 |
| TpoR(N2 + 1). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETLISLVTAL | |
| (775-825) | HLVLGLSAVLGLLLLRWQFPAHYRRLRHALWPSLPDLHRVL | |
| | GQYLRDTAALSPPKATVSDTCEEVEPSLLEILPKSSERTPLPLL | |
| | EARDEVEGFLQDTFPQQLEESEKQRLGGDVQSPNCPSEDVVI | |
| | TPESFGRDSSLTCLAGNVSACDAPILSSSRSLDCRESGKNGPH | |
| | VYQDLLLSLGTTNSTLPPPFSLQSGILTLNPVAQGQPILTSLGS | |
| | NQEEAYVTMSSFYQNQGSGSRSDPKPENPACPWTVLPAGDL | |
| | PTHDGYLPSNIDDLPSHEAPLADSLEELEPQ | |
| HAPD1(21-170). | PGWFLDSPDRPWNPPTFSPALLVVTEGDNATFTCSFSNTSESF | 181 |
| TpoR(N-3). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETSLVTALHL | |
| (775-825) | VLGLSAVLGLLLLRWQFPAHYRRLRHALWPSLPDLHRVLGQ | |
| | YLRDTAALSPPKATVSDTCEEVEPSLLEILPKSSERTPLPLLEA | |
| | RDEVEGFLQDTFPQQLEESEKQRLGGDVQSPNCPSEDVVITP | |
| | ESFGRDSSLTCLAGNVSACDAPILSSSRSLDCRESGKNGPHVY | |
| | QDLLLSLGTTNSTLPPPFSLQSGILTLNPVAQGQPILTSLGSNQ | |
| | EEAYVTMSSFYQNQGSGSRSDPKPENPACPWTVLPAGDLPT | |
| | HDGYLPSNIDDLPSHEAPLADSLEELEPQ | |
| HAPD1(21-170). | PGWFLDSPDRPWNPPTFSPALLVVTEGDNATFTCSFSNTSESF | 182 |
| TpoR(N-4). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETLVTALHL | |
| (775-825) | VLGLSAVLGLLLLRWQFPAHYRRLRHALWPSLPDLHRVLGQ | |
| | YLRDTAALSPPKATVSDTCEEVEPSLLEILPKSSERTPLPLLEA | |
| | RDEVEGFLQDTFPQQLEESEKQRLGGDVQSPNCPSEDVVITP | |
| | ESFGRDSSLTCLAGNVSACDAPILSSSRSLDCRESGKNGPHVY | |
| | QDLLLSLGTTNSTLPPPFSLQSGILTLNPVAQGQPILTSLGSNQ | |
| | EEAYVTMSSFYQNQGSGSRSDPKPENPACPWTVLPAGDLPT | |
| | HDGYLPSNIDDLPSHEAPLADSLEELEPQ | |
| HAPD1(21-170). | PGWFLDSPDRPWNPPTFSPALLVVTEGDNATFTCSFSNTSESF | 183 |
| TpoR(N-4 + 1). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETILVTALHL | |
| (775-825) | VLGLSAVLGLLLLRWQFPAHYRRLRHALWPSLPDLHRVLGQ | |
| | YLRDTAALSPPKATVSDTCEEVEPSLLEILPKSSERTPLPLLEA | |
| A | RDEVEGFLQDTFPQQLEESEKQRLGGDVQSPNCPSEDVVITP | |
| | ESFGRDSSLTCLAGNVSACDAPILSSSRSLDCRESGKNGPHVY | |
| | QDLLLSLGTTNSTLPPPFSLQSGILTLNPVAQGQPILTSLGSNQ | |
| | EEAYVTMSSFYQNQGSGSRSDPKPENPACPWTVLPAGDLPT | |
| | HDGYLPSNIDDLPSHEAPLADSLEELEPQ | |
| HAPD1(21-170). | PGWFLDSPDRPWNPPTFSPALLVVTEGDNATFTCSFSNTSESF | 184 |
| TpoR(N-5). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETVTALHLV | |
| (775-825) | LGLSAVLGLLLLRWQFPAHYRRLRHALWPSLPDLHRVLGQY | |
| | LRDTAALSPPKATVSDTCEEVEPSLLEILPKSSERTPLPLLEAR | |
| | DEVEGFLQDTFPQQLEESEKQRLGGDVQSPNCPSEDVVITPES | |
| | FGRDSSLTCLAGNVSACDAPILSSSRSLDCRESGKNGPHVYQ | |
| | DLLLSLGTTNSTLPPPFSLQSGILTLNPVAQGQPILTSLGSNQE | |
| | EAYVTMSSFYQNQGSGSRSDPKPENPACPWTVLPAGDLPTH | |
| | DGYLPSNIDDLPSHEAPLADSLEELEPQ | |
| HAPD1(21-170). | PGWFLDSPDRPWNPPTFSPALLVVTEGDNATFTCSFSNTSESF | 185 |
| TpoR(N-6). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETTALHLVL | |
| (775-825) | GLSAVLGLLLLRWQFPAHYRRLRHALWPSLPDLHRVLGQYL | |
| | RDTAALSPPKATVSDTCEEVEPSLLEILPKSSERTPLPLLEARD | |
| | EVEGFLQDTFPQQLEESEKQRLGGDVQSPNCPSEDVVITPESF | |
| | GRDSSLTCLAGNVSACDAPILSSSRSLDCRESGKNGPHVYQD | |
| | LLLSLGTTNSTLPPPFSLQSGILTLNPVAQGQPILTSLGSNQEE | |
| | AYVTMSSFYQNQGSGSRSDPKPENPACPWTVLPAGDLPTHD | |
| | GYLPSNIDDLPSHEAPLADSLEELEPQ | |
| HAPD1(21-170). | PGWFLDSPDRPWNPPTFSPALLVVTEGDNATFTCSFSNTSESF | 186 |
| TpoR(N-7). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETALHLVLG | |
| (775-825) | LSAVLGLLLLRWQFPAHYRRLRHALWPSLPDLHRVLGQYLR | |
| | DTAALSPPKATVSDTCEEVEPSLLEILPKSSERTPLPLLEARDE | |
| | VEGFLQDTFPQQLEESEKQRLGGDVQSPNCPSEDVVITPESFG | |
| | RDSSLTCLAGNVSACDAPILSSSRSLDCRESGKNGPHVYQDL | |
| | LLSLGTTNSTLPPPFSLQSGILTLNPVAQGQPILTSLGSNQEEA | |
| | YVTMSSFYQNQGSGSRSDPKPENPACPWTVLPAGDLPTHDG | |
| | YLPSNIDDLPSHEAPLADSLEELEPQ | |
| HAPD1(21-170). | PGWFLDSPDRPWNPPTFSPALLVVTEGDNATFTCSFSNTSESF | 187 |
| TpoR(N-8). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETLHLVLGLS | |
| (775-825) | AVLGLLLLRWQFPAHYRRLRHALWPSLPDLHRVLGQYLRDT | |
| | AALSPPKATVSDTCEEVEPSLLEILPKSSERTPLPLLEARDEVE | |
| | GFLQDTFPQQLEESEKQRLGGDVQSPNCPSEDVVITPESFGRD | |
| | SSLTCLAGNVSACDAPILSSSRSLDCRESGKNGPHVYQDLLLS | |
| | LGTTNSTLPPPFSLQSGILTLNPVAQGQPILTSLGSNQEEAYVT | |
| | MSSFYQNQGSGSRSDPKPENPACPWTVLPAGDLPTHDGYLP | |
| | SNIDDLPSHEAPLADSLEELEPQ | |
| HAPD1(21-170). | PGWFLDSPDRPWNPPTFSPALLVVTEGDNATFTCSFSNTSESF | 188 |
| TpoR(N9). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETHLVLGLS | |
| (775-825) | AVLGLLLLRWQFPAHYRRLRHALWPSLPDLHRVLGQYLRDT | |
| | AALSPPKATVSDTCEEVEPSLLEILPKSSERTPLPLLEARDEVE | |
| | GFLQDTFPQQLEESEKQRLGGDVQSPNCPSEDVVITPESFGRD | |
| | SSLTCLAGNVSACDAPILSSSRSLDCRESGKNGPHVYQDLLLS | |
| | LGTTNSTLPPPFSLQSGILTLNPVAQGQPILTSLGSNQEEAYVT | |
| | MSSFYQNQGSGSRSDPKPENPACPWTVLPAGDLPTHDGYLP | |
| | SNIDDLPSHEAPLADSLEELEPQ | |
| HAPD1(21-170). | PGWFLDSPDRPWNPPTFSPALLVVTEGDNATFTCSFSNTSESF | 189 |
| TpoR(N10). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETLVLGLSA | |
| (775-825) | VLGLLLLRWQFPAHYRRLRHALWPSLPDLHRVLGQYLRDTA | |
| | ALSPPKATVSDTCEEVEPSLLEILPKSSERTPLPLLEARDEVEG | |
| | FLQDTFPQQLEESEKQRLGGDVQSPNCPSEDVVITPESFGRDS | |
| | SLTCLAGNVSACDAPILSSSRSLDCRESGKNGPHVYQDLLLS | |
| | LGTTNSTLPPPFSLQSGILTLNPVAQGQPILTSLGSNQEEAYVT | |
| | MSSFYQNQGSGSRSDPKPENPACPWTVLPAGDLPTHDGYLP | |
| | SNIDDLPSHEAPLADSLEELEPQ | |
| HAPD1(21-170). | PGWFLDSPDRPWNPPTFSPALLVVTEGDNATFTCSFSNTSESF | 190 |
| TpoR(N-11). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETVLGLSAV | |
| (775-825) | LGLLLLRWQFPAHYRRLRHALWPSLPDLHRVLGQYLRDTAA | |
| | LSPPKATVSDTCEEVEPSLLEILPKSSERTPLPLLEARDEVEGF | |
| | LQDTFPQQLEESEKQRLGGDVQSPNCPSEDVVITPESFGRDSS | |
| | LTCLAGNVSACDAPILSSSRSLDCRESGKNGPHVYQDLLLSL | |
| | GTTNSTLPPPFSLQSGILTLNPVAQGQPILTSLGSNQEEAYVT | |
| | MSSFYQNQGSGSRSDPKPENPACPWTVLPAGDLPTHDGYLP | |
| | SNIDDLPSHEAPLADSLEELEPQ | |
| HAPD1(21-170). | PGWFLDSPDRPWNPPTFSPALLVVTEGDNATFTCSFSNTSESF | 191 |
| TpoR(N-12). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETLGLSAVL | |
| (775-825) | GLLLLRWQFPAHYRRLRHALWPSLPDLHRVLGQYLRDTAAL | |
| | SPPKATVSDTCEEVEPSLLEILPKSSERTPLPLLEARDEVEGFL | |
| | QDTFPQQLEESEKQRLGGDVQSPNCPSEDVVITPESFGRDSSL | |
| | TCLAGNVSACDAPILSSSRSLDCRESGKNGPHVYQDLLLSLG | |
| | TTNSTLPPPFSLQSGILTLNPVAQGQPILTSLGSNQEEAYVTM | |
| | SSFYQNQGSGSRSDPKPENPACPWTVLPAGDLPTHDGYLPSN | |
| | IDDLPSHEAPLADSLEELEPQ | |
| HAPD1(21-170). | PGWFLDSPDRPWNPPTFSPALLVVTEGDNATFTCSFSNTSESF | 192 |
| TpoR(N-13). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETGLSAVLG | |
| (775-825) | LLLLRWQFPAHYRRLRHALWPSLPDLHRVLGQYLRDTAALS | |
| | PPKATVSDTCEEVEPSLLEILPKSSERTPLPLLEARDEVEGFLQ | |
| | DTFPQQLEESEKQRLGGDVQSPNCPSEDVVITPESFGRDSSLT | |
| | CLAGNVSACDAPILSSSRSLDCRESGKNGPHVYQDLLLSLGT | |
| | TNSTLPPPFSLQSGILTLNPVAQGQPILTSLGSNQEEAYVTMSS | |
| | FYQNQGSGSRSDPKPENPACPWTVLPAGDLPTHDGYLPSNID | |
| | DLPSHEAPLADSLEELEPQ | |
| HAPD1(21-170). | PGWFLDSPDRPWNPPTFSPALLVVTEGDNATFTCSFSNTSESF | 193 |
| TpoR(N14). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETLSAVLGLL | |
| (775-825) | LLRWQFPAHYRRLRHALWPSLPDLHRVLGQYLRDTAALSPP | |
| | KATVSDTCEEVEPSLLEILPKSSERTPLPLLEARDEVEGFLQD | |
| | TFPQQLEESEKQRLGGDVQSPNCPSEDVVITPESFGRDSSLTC | |
| | LAGNVSACDAPILSSSRSLDCRESGKNGPHVYQDLLLSLGTT | |
| | NSTLPPPFSLQSGILTLNPVAQGQPILTSLGSNQEEAYVTMSSF | |
| | YQNQGSGSRSDPKPENPACPWTVLPAGDLPTHDGYLPSNIDD | |
| | LPSHEAPLADSLEELEPQ | |
| HAPD1(21-170). | PGWFLDSPDRPWNPPTFSPALLVVTEGDNATFTCSFSNTSESF | 194 |
| TpoR(N-15). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETSAVLGLLL | |
| (775-825) | LRWQFPAHYRRLRHALWPSLPDLHRVLGQYLRDTAALSPPK | |
| | ATVSDTCEEVEPSLLEILPKSSERTPLPLLEARDEVEGFLQDTF | |
| | PQQLEESEKQRLGGDVQSPNCPSEDVVITPESFGRDSSLTCLA | |
| | GNVSACDAPILSSSRSLDCRESGKNGPHVYQDLLLSLGTTNS | |
| | TLPPPFSLQSGILTLNPVAQGQPILTSLGSNQEEAYVTMSSFY | |
| | QNQGSGSRSDPKPENPACPWTVLPAGDLPTHDGYLPSNIDDL | |
| | PSHEAPLADSLEELEPQ | |
| HAPD1(21-170). | PGWFLDSPDRPWNPPTFSPALLVVTEGDNATFTCSFSNTSESF | 195 |
| TpoR(N16). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETAVLGLLLL | |
| (775-825) | RWQFPAHYRRLRHALWPSLPDLHRVLGQYLRDTAALSPPKA | |
| | TVSDTCEEVEPSLLEILPKSSERTPLPLLEARDEVEGFLQDTFP | |
| | QQLEESEKQRLGGDVQSPNCPSEDVVITPESFGRDSSLTCLAG | |
| | NVSACDAPILSSSRSLDCRESGKNGPHVYQDLLLSLGTTNSTL | |
| | PPPFSLQSGILTLNPVAQGQPILTSLGSNQEEAYVTMSSFYQN | |
| | QGSGSRSDPKPENPACPWTVLPAGDLPTHDGYLPSNIDDLPS | |
| | HEAPLADSLEELEPQ | |
| HAPD1(21-170). | PGWFLDSPDRPWNPPTFSPALLVVTEGDNATFTCSFSNTSESF | 196 |
| TpoR(N-17). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETVLGLLLLR | |
| (775-825) | WQFPAHYRRLRHALWPSLPDLHRVLGQYLRDTAALSPPKAT | |
| | VSDTCEEVEPSLLEILPKSSERTPLPLLEARDEVEGFLQDTFPQ | |
| | QLEESEKQRLGGDVQSPNCPSEDVVITPESFGRDSSLTCLAGN | |
| | VSACDAPILSSSRSLDCRESGKNGPHVYQDLLLSLGTTNSTLP | |
| | PPFSLQSGILTLNPVAQGQPILTSLGSNQEEAYVTMSSFYQNQ | |
| | GSGSRSDPKPENPACPWTVLPAGDLPTHDGYLPSNIDDLPSH | |
| | EAPLADSLEELEPQ | |
| HAPD1(21-170). | PGWFLDSPDRPWNPPTFSPALLVVTEGDNATFTCSFSNTSESF | 197 |
| TpoR(N-18). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETLGLLLLR | |
| (775-825) | WQFPAHYRRLRHALWPSLPDLHRVLGQYLRDTAALSPPKAT | |
| | VSDTCEEVEPSLLEILPKSSERTPLPLLEARDEVEGFLQDTFPQ | |
| | QLEESEKQRLGGDVQSPNCPSEDVVITPESFGRDSSLTCLAGN | |
| | VSACDAPILSSSRSLDCRESGKNGPHVYQDLLLSLGTTNSTLP | |
| | PPFSLQSGILTLNPVAQGQPILTSLGSNQEEAYVTMSSFYQNQ | |
| | GSGSRSDPKPENPACPWTVLPAGDLPTHDGYLPSNIDDLPSH | |
| | EAPLADSLEELEPQ | |
| HAPD1(21-170). | PGWFLDSPDRPWNPPTFSPALLVVTEGDNATFTCSFSNTSESF | 198 |
| TpoR(N + 1). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETAWLISLVT | |
| (775-825) | ALHLVLGLSAVLGLLLLRWQFPAHYRRLRHALWPSLPDLHR | |
| | VLGQYLRDTAALSPPKATVSDTCEEVEPSLLEILPKSSERTPL | |
| | PLLEARDEVEGFLQDTFPQQLEESEKQRLGGDVQSPNCPSED | |
| | VVITPESFGRDSSLTCLAGNVSACDAPILSSSRSLDCRESGKN | |
| | GPHVYQDLLLSLGTTNSTLPPPFSLQSGILTLNPVAQGQPILTS | |
| | LGSNQEEAYVTMSSFYQNQGSGSRSDPKPENPACPWTVLPA | |
| | GDLPTHDGYLPSNIDDLPSHEAPLADSLEELEPQ | |
| HAPD1(21-170). | PGWFLDSPDRPWNPPTFSPALLVVTEGDNATFTCSFSNTSESF | 199 |
| TpoR(N + 2). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETAWVLISLV | |
| (775-825) | TALHLVLGLSAVLGLLLLRWQFPAHYRRLRHALWPSLPDLH | |
| | RVLGQYLRDTAALSPPKATVSDTCEEVEPSLLEILPKSSERTP | |
| | LPLLEARDEVEGFLQDTFPQQLEESEKQRLGGDVQSPNCPSE | |
| | DVVITPESFGRDSSLTCLAGNVSACDAPILSSSRSLDCRESGK | |
| | NGPHVYQDLLLSLGTTNSTLPPPFSLQSGILTLNPVAQGQPIL | |
| | TSLGSNQEEAYVTMSSFYQNQGSGSRSDPKPENPACPWTVLP | |
| | AGDLPTHDGYLPSNIDDLPSHEAPLADSLEELEPQ | |
| HAPD1(21-170). | PGWFLDSPDRPWNPPTFSPALLVVTEGDNATFTCSFSNTSESF | 200 |
| TpoR(N + 3). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETAWLVLISL | |
| (775-825) | VTALHLVLGLSAVLGLLLLRWQFPAHYRRLRHALWPSLPDL | |
| | HRVLGQYLRDTAALSPPKATVSDTCEEVEPSLLEILPKSSERT | |
| | PLPLLEARDEVEGFLQDTFPQQLEESEKQRLGGDVQSPNCPS | |
| | EDVVITPESFGRDSSLTCLAGNVSACDAPILSSSRSLDCRESG | |
| | KNGPHVYQDLLLSLGTTNSTLPPPFSLQSGILTLNPVAQGQPI | |
| | LTSLGSNQEEAYVTMSSFYQNQGSGSRSDPKPENPACPWTV | |
| | LPAGDLPTHDGYLPSNIDDLPSHEAPLADSLEELEPQ | |
| HAPD1(21-170). | PGWFLDSPDRPWNPPTFSPALLVVTEGDNATFTCSFSNTSESF | 201 |
| TpoR(N + 4). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETAWILVLIS | |
| (775-825) | LVTALHLVLGLSAVLGLLLLRWQFPAHYRRLRHALWPSLPD | |
| | LHRVLGQYLRDTAALSPPKATVSDTCEEVEPSLLEILPKSSER | |
| | TPLPLLEARDEVEGFLQDTFPQQLEESEKQRLGGDVQSPNCP | |
| | SEDVVITPESFGRDSSLTCLAGNVSACDAPILSSSRSLDCRESG | |
| | KNGPHVYQDLLLSLGTTNSTLPPPFSLQSGILTLNPVAQGQPI | |
| | LTSLGSNQEEAYVTMSSFYQNQGSGSRSDPKPENPACPWTV | |
| | LPAGDLPTHDGYLPSNIDDLPSHEAPLADSLEELEPQ | |
| HAPD1(21-170). | PGWFLDSPDRPWNPPTFSPALLVVTEGDNATFTCSFSNTSESF | 202 |
| TpoR(N + 5). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETAWLILVLI | |
| (775-825) | SLVTALHLVLGLSAVLGLLLLRWQFPAHYRRLRHALWPSLP | |
| | DLHRVLGQYLRDTAALSPPKATVSDTCEEVEPSLLEILPKSSE | |
| | RTPLPLLEARDEVEGFLQDTFPQQLEESEKQRLGGDVQSPNC | |
| | PSEDVVITPESFGRDSSLTCLAGNVSACDAPILSSSRSLDCRES | |
| | GKNGPHVYQDLLLSLGTTNSTLPPPFSLQSGILTLNPVAQGQP | |
| | ILTSLGSNQEEAYVTMSSFYQNQGSGSRSDPKPENPACPWTV | |
| | LPAGDLPTHDGYLPSNIDDLPSHEAPLADSLEELEPQ | |
| HAPD1(21-170). | PGWFLDSPDRPWNPPTFSPALLVVTEGDNATFTCSFSNTSESF | 203 |
| TpoR(N + 6). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETAWLLILVL | |
| (775-825) | ISLVTALHLVLGLSAVLGLLLLRWQFPAHYRRLRHALWPSLP | |
| | DLHRVLGQYLRDTAALSPPKATVSDTCEEVEPSLLEILPKSSE | |
| | RTPLPLLEARDEVEGFLQDTFPQQLEESEKQRLGGDVQSPNC | |
| | PSEDVVITPESFGRDSSLTCLAGNVSACDAPILSSSRSLDCRES | |
| | GKNGPHVYQDLLLSLGTTNSTLPPPFSLQSGILTLNPVAQGQP | |
| | ILTSLGSNQEEAYVTMSSFYQNQGSGSRSDPKPENPACPWTV | |
| | LPAGDLPTHDGYLPSNIDDLPSHEAPLADSLEELEPQ | |
| HAPD1(21-170). | PGWFLDSPDRPWNPPTFSPALLVVTEGDNATFTCSFSNTSESF | 204 |
| TpoR(N + 7). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETAWVLLIL | |
| (775-825) | VLISLVTALHLVLGLSAVLGLLLLRWQFPAHYRRLRHALWP | |
| | SLPDLHRVLGQYLRDTAALSPPKATVSDTCEEVEPSLLEILPK | |
| | SSERTPLPLLEARDEVEGFLQDTFPQQLEESEKQRLGGDVQSP | |
| | NCPSEDVVITPESFGRDSSLTCLAGNVSACDAPILSSSRSLDCR | |
| | ESGKNGPHVYQDLLLSLGTTNSTLPPPFSLQSGILTLNPVAQG | |
| | QPILTSLGSNQEEAYVTMSSFYQNQGSGSRSDPKPENPACPW | |
| | TVLPAGDLPTHDGYLPSNIDDLPSHEAPLADSLEELEPQ | |
| HAPD1(21-170). | PGWFLDSPDRPWNPPTFSPALLVVTEGDNATFTCSFSNTSESF | 205 |
| TpoR(N + 8). | HVIWHRESPSGQTDTLAAFPEDRSQPGQDCRFRVTQLPNGRD | |
| IL7Ra(316-459). | FHMSVVRARRNDSGTYVCGVISLAPKIQIKESLRAELRVTER | |
| IL12Rb2 | RAEVPTAHPSPSPRPAGQFQTLVSDPTRVETATETAWLVLLIL | |
| (775-825) | VLISLVTALHLVLGLSAVLGLLLLRWQFPAHYRRLRHALWP | |
| | SLPDLHRVLGQYLRDTAALSPPKATVSDTCEEVEPSLLEILPK | |
| | SSERTPLPLLEARDEVEGFLQDTFPQQLEESEKQRLGGDVQSP | |
| | NCPSEDVVITPESFGRDSSLTCLAGNVSACDAPILSSSRSLDCR | |
| | ESGKNGPHVYQDLLLSLGTTNSTLPPPFSLQSGILTLNPVAQG | |
| | QPILTSLGSNQEEAYVTMSSFYQNQGSGSRSDPKPENPACPW | |
| | TVLPAGDLPTHDGYLPSNIDDLPSHEAPLADSLEELEPQ | |
| HAPD1(21-107). | PGWFLDSPDRPWNPPTFSPALLVVTEGDNATFTCSFSNTSESF | 206 |
| TPOR/ | HVIWHRESPSGQTDTLAAFPEDRSQPGQDCRFRVTQLPNGRD | |
| MPLR(478-582; | FHMSVVRARRNDSGTYVCGVISLAPKIQIKESLRAELRVTER | |
| H499L, S505N, | RAEVPTAHPSPSPRPAGQFQTLVSDPTRVETATETAWISLVTA | |
| W515K). | LLLVLGLNAVLGLLLLRKQFPAHYRRLRHALWPSLPDLHRV | |
| IL7Ra(316-459) | LGQYLRDTAALSPPKATVSDTCEEVEPSLLEILPKSSERTPLPL | |
| | LEARDEVEGFLQDTFPQQLEESEKQRLGGDVQSPNCPSEDVV | |
| | ITPESFGRDSSLTCLAGNVSACDAPILSSSRSLDCRESGKNGP | |
| | HVYQDLLLSLGTTNSTLPPPFSLQSGILTLNPVAQGQPILTSLG | |
| | SNQEEAYVTMSSFYQNQ | |
| HAPD1.TpoR | PGWFLDSPDRPWNPPTFSPALLVVTEGDNATFTCSFSNTSESF | 207 |
| (478-582; S505N, | HVIWHRESPSGQTDTLAAFPEDRSQPGQDCRFRVTQLPNGRD | |
| W515K). | FHMSVVRARRNDSGTYVCGVISLAPKIQIKESLRAELRVTER | |
| IL2Rbsmall | RAEVPTAHPSPSPRPAGQFQTLVSDPTRVETATETAWISLVTA | |
| (393-433, 518- | LHLVLGLNAVLGLLLLRKQFPAHYRRLRHALWPSLPDLHRV | |
| 551) | LGQYLRDTAALSPPKATVSDTCEEVEPSLLEILPKSSERTPLPL | |
| | LEDEGVAGAPTGSSPQPLQPLSGEDDAYCTFPSRDDLLLFSPS | |
| | GQGEFRALNARLPLNTDAYLSLQELQGQDPTHLV | |
| HAPD1(21-107). | PGWFLDSPDRPWNPPTFSPALLVVTEGDNATFTCSFSNTSESF | 208 |
| TpoR(478-582). | HVIWHRESPSGQTDTLAAFPEDRSQPGQDCRFRVTQLPNGRD | |
| IL7Ra(316-459) | FHMSVVRARRNDSGTYVCGVISLAPKIQIKESLRAELRVTER | |
| | RAEVPTAHPSPSPRPAGQFQTLVSDPTRVETATETAWISLVTA | |
| | LHLVLGLSAVLGLLLLRWQFPAHYRRLRHALWPSLPDLHRV | |
| | LGQYLRDTAALSPPKATVSDTCEEVEPSLLEILPKSSERTPLPL | |
| | LEARDEVEGFLQDTFPQQLEESEKQRLGGDVQSPNCPSEDVV | |
| | ITPESFGRDSSLTCLAGNVSACDAPILSSSRSLDCRESGKNGP | |
| | HVYQDLLLSLGTTNSTLPPPFSLQSGILTLNPVAQGQPILTSLG | |
| | SNQEEAYVTMSSFYQNQ | |
| HAPD1.TpoR | PGWFLDSPDRPWNPPTFSPALLVVTEGDNATFTCSFSNTSESF | 209 |
| (478-582). | HVIWHRESPSGQTDTLAAFPEDRSQPGQDCRFRVTQLPNGRD | |
| IL2Rbsmall(393- | FHMSVVRARRNDSGTYVCGVISLAPKIQIKESLRAELRVTER | |
| 433, 518-551) | RAEVPTAHPSPSPRPAGQFQTLVSDPTRVETATETAWISLVTA | |
| | LHLVLGLSAVLGLLLLRWQFPAHYRRLRHALWPSLPDLHRV | |
| | LGQYLRDTAALSPPKATVSDTCEEVEPSLLEILPKSSERTPLPL | |
| | LEDEGVAGAPTGSSPQPLQPLSGEDDAYCTFPSRDDLLLFSPS | |
| | GQGEFRALNARLPLNTDAYLSLQELQGQDPTHLV | |
| HAPD1(21-107) | PGWFLDSPDRPWNPPTFSPALLVVTEGDNATFTCSFSNTSESF | 214 |
| TpoR(478-582; | HVIWHRESPSGQTDTLAAFPEDRSQPGQDCRFRVTQLPNGRD | |
| S505N, W515K). | FHMSVVRARRNDSGTYVCGVISLAPKIQIKESLRAELRVTER | |
| IL2Rbsmall | RAEVPTAHPSPSPRPAGQFQTLVSDPTRVETATETAWISLVTA | |
| (339-379, 393- | LHLVLGLNAVLGLLLLRKQFPAHYRRLRHALWPSLPDLHRV | |
| 433, 518-551) | LGQYLRDTAALSPPKATVSDTCEEVEPSLLEILPKSSERTPLPL | |
| | LEQQDKVPEPASLSSNHSLTSCFTNQGYFFFHLPDALEIEACQ | |
| | DEGVAGAPTGSSPQPLQPLSGEDDAYCTFPSRDDLLLFSPSG | |
| | QGEFRALNARLPLNTDAYLSLQELQGQDPTHLV | |
| HAPD1(21-107). | PGWFLDSPDRPWNPPTFSPALLVVTEGDNATFTCSFSNTSESF | 215 |
| TpoR(478-582; | HVIWHRESPSGQTDTLAAFPEDRSQPGQDCRFRVTQLPNGRD | |
| H499L, S505N, | FHMSVVRARRNDSGTYVCGVISLAPKIQIKESLRAELRVTER | |
| W515K). | RAEVPTAHPSPSPRPAGQFQTLVSDPTRVETATETAWISLVTA | |
| IL2Rbsmall | LLLVLGLNAVLGLLLLRKQFPAHYRRLRHALWPSLPDLHRV | |
| (339-379, 393- | LGQYLRDTAALSPPKATVSDTCEEVEPSLLEILPKSSERTPLPL | |
| 433, 518-551) | LEQQDKVPEPASLSSNHSLTSCFTNQGYFFFHLPDALEIEACQ | |
| | DEGVAGAPTGSSPQPLQPLSGEDDAYCTFPSRDDLLLFSPSG | |
| | QGEFRALNARLPLNTDAYLSLQELQGQDPTHLV | |
| HAPD1(21-170). | PGWFLDSPDRPWNPPTFSPALLVVTEGDNATFTCSFSNTSESF | 216 |
| TpoR(478-582). | HVIWHRESPSGQTDTLAAFPEDRSQPGQDCRFRVTQLPNGRD | |
| IL2Rbsmall | FHMSVVRARRNDSGTYVCGVISLAPKIQIKESLRAELRVTER | |
| (339-379, 393- | RAEVPTAHPSPSPRPAGQFQTLVSDPTRVETATETAWISLVTA | |
| 433, 518-551) | LHLVLGLSAVLGLLLLRWQFPAHYRRLRHALWPSLPDLHRV | |
| | LGQYLRDTAALSPPKATVSDTCEEVEPSLLEILPKSSERTPLPL | |
| | LEQQDKVPEPASLSSNHSLTSCFTNQGYFFFHLPDALEIEACQ | |
| | DEGVAGAPTGSSPQPLQPLSGEDDAYCTFPSRDDLLLFSPSG | |
| | QGEFRALNARLPLNTDAYLSLQELQGQDPTHLV | |
| HAPD1(21-107). | PGWFLDSPDRPWNPPTFSPALLVVTEGDNATFTCSFSNTSESF | 217 |
| TpoR(N-7). | HVIWHRESPSGQTDTLAAFPEDRSQPGQDCRFRVTQLPNGRD | |
| IL2Rbsmall | FHMSVVRARRNDSGTYVCGVISLAPKIQIKESLRAELRVTER | |
| (393-433, | RAEVPTAHPSPSPRPAGQFQTLVSDPTRVETATETALHLVLG | |
| 518-551) | LSAVLGLLLLRWQFPAHYRRLRHALWPSLPDLHRVLGQYLR | |
| | DTAALSPPKATVSDTCEEVEPSLLEILPKSSERTPLPLLEDEGV | |
| | AGAPTGSSPQPLQPLSGEDDAYCTFPSRDDLLLFSPSGQGEFR | |
| | ALNARLPLNTDAYLSLQELQGQDPTHLV | |
| HAPD1(21-107). | PGWFLDSPDRPWNPPTFSPALLVVTEGDNATFTCSFSNTSESF | 218 |
| TpoR(N-8). | HVIWHRESPSGQTDTLAAFPEDRSQPGQDCRFRVTQLPNGRD | |
| IL2Rbsmall | FHMSVVRARRNDSGTYVCGVISLAPKIQIKESLRAELRVTER | |
| (393- | RAEVPTAHPSPSPRPAGQFQTLVSDPTRVETATETLHLVLGLS | |
| 433, 518-551) | AVLGLLLLRWQFPAHYRRLRHALWPSLPDLHRVLGQYLRDT | |
| | AALSPPKATVSDTCEEVEPSLLEILPKSSERTPLPLLEDEGVAG | |
| | APTGSSPQPLQPLSGEDDAYCTFPSRDDLLLFSPSGQGEFRAL | |
| | NARLPLNTDAYLSLQELQGQDPTHLV | |
| HAPD1(21-107). | PGWFLDSPDRPWNPPTFSPALLVVTEGDNATFTCSFSNTSESF | 219 |
| TpoR(N-9). | HVIWHRESPSGQTDTLAAFPEDRSQPGQDCRFRVTQLPNGRD | |
| IL2Rbsmall | FHMSVVRARRNDSGTYVCGVISLAPKIQIKESLRAELRVTER | |
| (393- | RAEVPTAHPSPSPRPAGQFQTLVSDPTRVETATETHLVLGLS | |
| 433, 518-551) | AVLGLLLLRWQFPAHYRRLRHALWPSLPDLHRVLGQYLRDT | |
| | AALSPPKATVSDTCEEVEPSLLEILPKSSERTPLPLLEDEGVAG | |
| | APTGSSPQPLQPLSGEDDAYCTFPSRDDLLLFSPSGQGEFRAL | |
| | NARLPLNTDAYLSLQELQGQDPTHLV | |
| HAPD1(21-107). | PGWFLDSPDRPWNPPTFSPALLVVTEGDNATFTCSFSNTSESF | 220 |
| TpoR(N-7). | HVIWHRESPSGQTDTLAAFPEDRSQPGQDCRFRVTQLPNGRD | |
| IL2Rbsmall | FHMSVVRARRNDSGTYVCGVISLAPKIQIKESLRAELRVTER | |
| (339-379, 393- | RAEVPTAHPSPSPRPAGQFQTLVSDPTRVETATETALHLVLG | |
| 433, 518-551) | LSAVLGLLLLRWQFPAHYRRLRHALWPSLPDLHRVLGQYLR | |
| | DTAALSPPKATVSDTCEEVEPSLLEILPKSSERTPLPLLEQQDK | |
| | VPEPASLSSNHSLTSCFTNQGYFFFHLPDALEIEACQDEGVAG | |
| | APTGSSPQPLQPLSGEDDAYCTFPSRDDLLLFSPSGQGEFRAL | |
| | NARLPLNTDAYLSLQELQGQDPTHLV | |
| HAPD1(21-107). | PGWFLDSPDRPWNPPTFSPALLVVTEGDNATFTCSFSNTSESF | 221 |
| TpoR(N-8). | HVIWHRESPSGQTDTLAAFPEDRSQPGQDCRFRVTQLPNGRD | |
| IL2Rbsmall | FHMSVVRARRNDSGTYVCGVISLAPKIQIKESLRAELRVTER | |
| (339-379, 393- | RAEVPTAHPSPSPRPAGQFQTLVSDPTRVETATETLHLVLGLS | |
| 433, 518-551) | AVLGLLLLRWQFPAHYRRLRHALWPSLPDLHRVLGQYLRDT | |
| | AALSPPKATVSDTCEEVEPSLLEILPKSSERTPLPLLEQQDKVP | |
| | EPASLSSNHSLTSCFTNQGYFFFHLPDALEIEACQDEGVAGAP | |
| | TGSSPQPLQPLSGEDDAYCTFPSRDDLLLFSPSGQGEFRALNA | |
| | RLPLNTDAYLSLQELQGQDPTHLV | |
| HAPD1(21-107). | PGWFLDSPDRPWNPPTFSPALLVVTEGDNATFTCSFSNTSESF | 222 |
| TpoR(N-19). | HVIWHRESPSGQTDTLAAFPEDRSQPGQDCRFRVTQLPNGRD | |
| IL2Rbsmall | FHMSVVRARRNDSGTYVCGVISLAPKIQIKESLRAELRVTER | |
| (339-379, 393- | RAEVPTAHPSPSPRPAGQFQTLVSDPTRVETATETHLVLGLS | |
| 433, 518-551) | AVLGLLLLRWQFPAHYRRLRHALWPSLPDLHRVLGQYLRDT | |
| | AALSPPKATVSDTCEEVEPSLLEILPKSSERTPLPLLEQQDKVP | |
| | EPASLSSNHSLTSCFTNQGYFFFHLPDALEIEACQDEGVAGAP | |
| | TGSSPQPLQPLSGEDDAYCTFPSRDDLLLFSPSGQGEFRALNA | |
| | RLPLNTDAYLSLQELQGQDPTHLV | |
| HAPD1(21-170). | PGWFLDSPDRPWNPPTFSPALLVVTEGDNATFTCSFSNTSESF | 223 |
| TpoR(478-582; | HVIWHRESPSGQTDTLAAFPEDRSQPGQDCRFRVTQLPNGRD | |
| H499L, S505N, | FHMSVVRARRNDSGTYVCGVISLAPKIQIKESLRAELRVTER | |
| W515K). | RAEVPTAHPSPSPRPAGQFQTLVSDPTRVETATETAWISLVTA | |
| IL2Rbsmall | LLLVLGLNAVLGLLLLRKQFPAHYRRLRHALWPSLPDLHRV | |
| (393- | LGQYLRDTAALSPPKATVSDTCEEVEPSLLEILPKSSERTPLPL | |
| 433, 518-551) | LEDEGVAGAPTGSSPQPLQPLSGEDDAYCTFPSRDDLLLFSPS | |
| | GQGEFRALNARLPLNTDAYLSLQELQGQDPTHLV | |
| HAPD1(21-170). | PGWFLDSPDRPWNPPTFSPALLVVTEGDNATFTCSFSNTSESF | 224 |
| TpoR(478-582; | HVIWHRESPSGQTDTLAAFPEDRSQPGQDCRFRVTQLPNGRD | |
| S505N, W515K). | FHMSVVRARRNDSGTYVCGVISLAPKIQIKESLRAELRVTER | |
| IL7Ra(316-459) | RAEVPTAHPSPSPRPAGQFQTLVSDPTRVETATETAWISLVTA | |
| | LHLVLGLNAVLGLLLLRKQFPAHYRRLRHALWPSLPDLHRV | |
| | LGQYLRDTAALSPPKATVSDTCEEVEPSLLEILPKSSERTPLPL | |
| | LEARDEVEGFLQDTFPQQLEESEKQRLGGDVQSPNCPSEDVV | |
| | ITPESFGRDSSLTCLAGNVSACDAPILSSSRSLDCRESGKNGP | |
| | HVYQDLLLSLGTTNSTLPPPFSLQSGILTLNPVAQGQPILTSLG | |
| | SNQEEAYVTMSSFYQNQ | |

### F. Expression of PD-1 Chimeric Cytokine Receptors

Provided herein are polynucleotides encoding any one of the chimeric cytokine receptors provided herein. Likewise, provided herein are expression vectors comprising such polynucleotides. In some embodiments, the vector is a viral vector. In some embodiments, the vector is not a viral vector.

In some embodiments, the vector comprises a polynucleotide encoding a PD-1 chimeric cytokine receptor, and a polynucleotide expressing a chimeric antigen receptor (CAR).

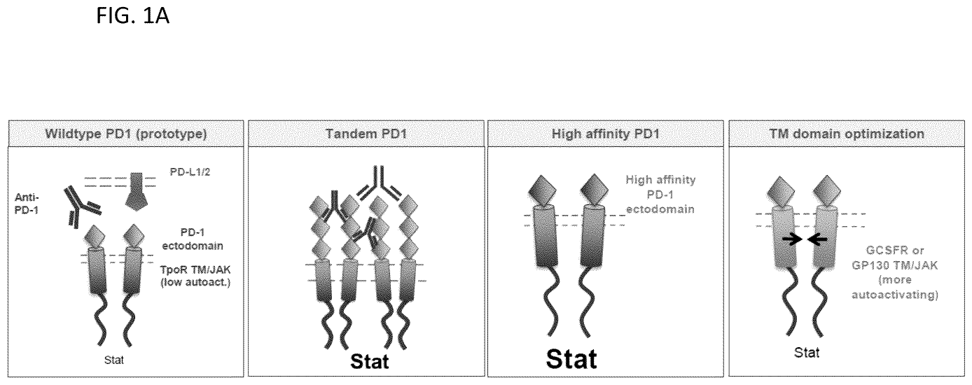

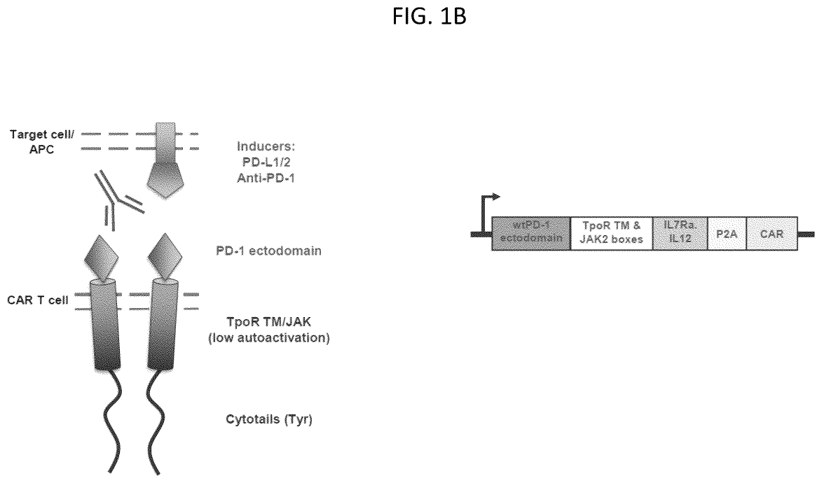

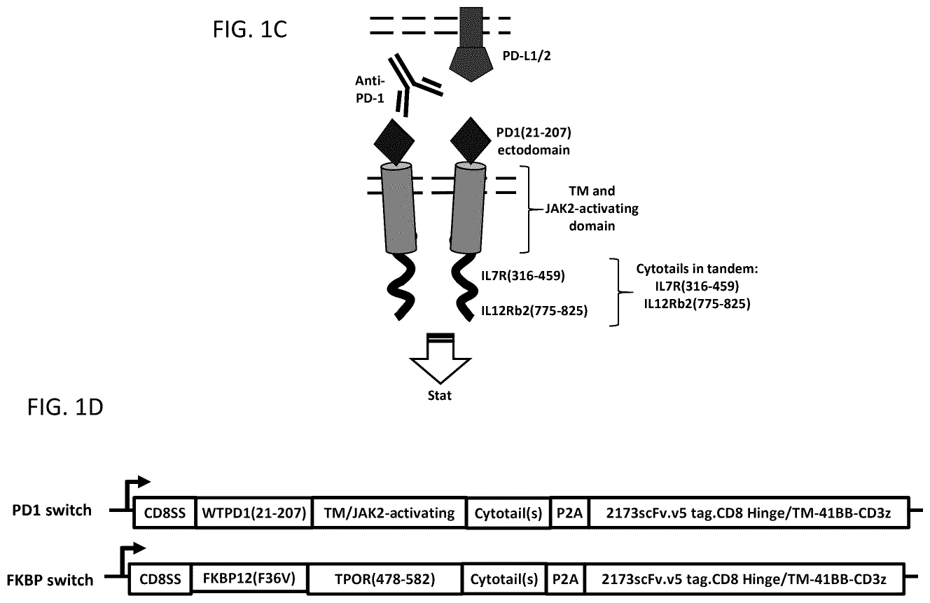

In some embodiments, expression of the chimeric cytokine receptor and the CAR are expressed as a single polypeptide chain, separated by a linker. FIGS. 1B and 1D show schematics of a vector that can be used to co-express the chimeric cytokine receptor and CAR of the disclosure. One or more STAT-recruiting domains may be joined in tandem to mimic signaling from one or more cytokines.

### II. CAR-Bearing Immune Cells

Provided herein are engineered immune cells comprising a polynucleotide encoding a chimeric antigen receptor and a PD-1 chimeric cytokine receptor of the disclosure; and provided herein are engineered immune cells expressing a chimeric antigen receptor (CAR-I cell) and a PD-1 chimeric cytokine receptor of the disclosure. Examples of immune cells include T-cells, e.g., alpha/beta T-cells and gamma/delta T-cells, B cells, natural killer (NK) cells, natural killer T (NKT) cells, invariant NKT cells, mast cells, myeloic-derived phagocytes, dendritic cells, killer dendritic cells, macrophages, and monocytes. Immune cells also refer to cells derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells.

Accordingly in some embodiments, provided herein are CAR-T-cells comprising a PD-1 chimeric cytokine receptor of the disclosure.

In some embodiments, a CAR can comprise an extracellular ligand-binding domain (e.g., a single chain variable fragment (scFv)), a transmembrane domain, and an intracellular signaling domain. In some embodiments, the extracellular ligand-binding domain, transmembrane domain, and intracellular signaling domain are in one polypeptide, i.e., in a single chain. Multichain CARs and polypeptides are also provided herein. In some embodiments, the multichain CARs comprise: a first polypeptide comprising a transmembrane domain and at least one extracellular ligand-binding domain, and a second polypeptide comprising a transmembrane domain and at least one intracellular signaling domain, wherein the polypeptides assemble together to form a multichain CAR.

The extracellular ligand-binding domain of a CAR specifically binds to a target of interest. The target of interest can be any molecule of interest, including, for example, without limitation any one or more of those presented in Table 11.

TABLE 11
List of targets of interest

BCMA
EGFRvIII
Flt-3
WT-1
CD20
CD23
CD30
CD38
CD70
CD33
CD133
MHC- WT1
TSPAN10
MHC-PRAME
Liv1
ADAM10
CHRNA2
LeY
NKG2D
CS1
CD44v6
ROR1
CD19
Claudin-18.2 (Claudin-18A2 or Claudin18 isoform 2)
DLL3 (Delta-like protein 3, Drosophila Delta homolog 3, Delta3)
Muc17 (Mucin17, Muc3, Muc3)
FAP alpha (Fibroblast Activation Protein alpha)
Ly6G6D (Lymphocyte antigen 6 complex locus protein G6d
c6orf23
G6D
MEGT1
NG25)
RNF43 (E3 ubiquitin-protein ligase RNF43, RING finger protein 43)
ErbB2 (HER2/neu)
carcinoembryonic antigen (CEA)
epithelial cell adhesion molecule (EpCAM)
epidermal growth factor receptor (EGFR)
CD40
disialoganglioside GD2
GD3
C-type lectin-like molecule-1 (CLL-1)
ductal-epithelial mucine
gp36
TAG-72
glycosphingolipids
glioma-associated antigen
β-human chorionic gonadotropin
alphafetoprotein (AFP)
lectin-reactive AFP
thyroglobulin
RAGE-1
MN-CA IX
human telomerase reverse transcriptase
RU1
RU2 (AS)
intestinal carboxyl esterase
mut hsp70-2
M-CSF
prostase
prostase specific antigen (PSA)
PAP
NY-ESO-1
LAGA-la
p53
prostein
PSMA
survivin and telomerase
prostate-carcinoma tumor antigen-1 (PCTA-1)
MAGE
ELF2M
neutrophil elastase
ephrin B2
CD22
insulin growth factor (IGFl)-l
IGF-II
IGFI receptor
mesothelin
a major histocompatibility complex (MHC) molecule presenting a
tumor-specific peptide epitope
5T4
O 1
Nkp30
tumor stromal antigens
the extra domain A (EDA) and extra domain B (EDB) of fibronectin and
the AI domain of tenascin-C (TnC AI) and fibroblast associated protein
(fap)
LRP6
melamona-associated Chondroitin Sulfate Proteoglycan (MCSP)
MARTI
MUC1
LMP2
Idiotype
NY-ESO-1
Ras mutant
gp100
proteinase 3
bcr-abl
tyrosinase
hTERT
EphA2
ML-TAP
ERG
NA17
PAX3
ALK
Androgen receptor
a lineage-specific or tissue specific antigen such as CD3
CD4
CD8
CD24
CD25
CD34
CD79
CD116
CD117
CD135
CD123
CD138
CTLA-4
B7-1 (CD80)
B7-2 (CD86)
endoglin
a major histocompatibility complex (MHC) molecule
MUC16
PSCA
Trop2
CD171 (L1CAM)
CA9
STEAP1
VEGFR2

In some embodiments, the extracellular ligand-binding domain of a CAR comprises an scFv comprising the light chain variable (VL) region and the heavy chain variable (VH) region of a target antigen specific monoclonal antibody joined by a flexible linker. Single chain variable region fragments are made by linking light and/or heavy chain variable regions by using a short linking peptide (Bird et al., Science 242:423-426, 1988) (e.g. glycine-serine containing linkers). In general, linkers can be short, flexible polypeptides and are generally comprised of about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The intracellular signaling domain of a CAR according to the invention is responsible for intracellular signaling following the binding of extracellular ligand-binding domain to the target resulting in the activation of the immune cell and immune response (Signals 1 and/or 2). The intracellular signaling domain has the ability to activate at least one of the normal effector functions of the immune cell in which the CAR is expressed. For example, the effector function of a T cell can be a cytolytic activity or helper activity including the secretion of cytokines.

In some embodiments, an intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability. Intracellular signaling domains comprise two distinct classes of cytoplasmic signaling sequences: those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non-limiting examples those derived from TCRζ, FcRγ, FcRβ, FcRε, CD3γ, CD3δ, CD3ε, CD5, CD22, CD79a, CD79b and CD66d. In some embodiments, the intracellular signaling domain of the CAR can comprise the CD3ζ signaling domain. In some embodiments the intracellular signaling domain of the CAR of the invention comprises a domain of a co-stimulatory molecule.

In some embodiments, the intracellular signaling domain of a CAR of the invention comprises a part of co-stimulatory molecule selected from the group consisting of fragment of 41BB (GenBank: AAA53133.) and CD28 (NP_006130.1).

CARs are expressed on the surface membrane of the cell. Thus, the CAR comprises a transmembrane domain. Suitable transmembrane domains for a CAR disclosed herein have the ability to (a) be expressed at the surface of a cell, preferably an immune cell such as, for example without limitation, lymphocyte cells or Natural killer (NK) cells, and (b) interact with the ligand-binding domain and intracellular signaling domain for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T cell receptor such as α, β, γ or δ, polypeptide constituting CD3 complex, IL-2 receptor p55 (a chain), p75 (β chain) or γ chain, subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1). The transmembrane domain can further comprise a stalk domain between the extracellular ligand-binding domain and said transmembrane domain. A stalk domain may comprise up to 300 amino acids, preferably 10 to 100 amino acids and most preferably 25 to 50 amino acids. Stalk region may be derived from all or part of naturally occurring molecules, such as from all or part of the extracellular region of CD8, CD4, or CD28, or from all or part of an antibody constant region. Alternatively the stalk domain may be a synthetic sequence that corresponds to a naturally occurring stalk sequence, or may be an entirely synthetic stalk sequence. In some embodiments said stalk domain is a part of human CD8α chain (e.g., NP_001139345.1). In another particular embodiment, said transmembrane and hinge domains comprise a part of human CD8α chain. In some embodiments, CARs disclosed herein can comprise an extracellular ligand-binding domain that specifically binds BCMA, CD8α human hinge and transmembrane domains, the CD3ζ signaling domain, and 4-1BB signaling domain.

In some embodiments, a CAR can be introduced into an immune cell as a transgene via a plasmid vector. In some embodiments, the plasmid vector can also contain, for example, a selection marker which provides for identification and/or selection of cells which received the vector.

Table 8 provides exemplary sequences of CAR components that can be used in the CARs disclosed herein.

TABLE 8
Exemplary Sequences

| | | SEQ |
|---|---|---|
| Domain | Amino acid sequence | ID NO: |
| V5 epitope tag | IPNPLLGLDST | 122 |
| 2173 scFv | EIQLVQSGAEVKKPGESLRISCKGSGFNIEDYYIH | 123 |
| | WVRQMPGKGLEWMGRIDPENDETKYGPIFQGH | |
| | VTISADTSINTVYLQWSSLKASDTAMYYCAFRG | |
| | GVYWGQGTTVTVSSGGGGSGGGGSGGGGSGGG | |
| | GSDVVMTQSPDSLAVSLGERATINCKSSQSLLDS | |
| | DGKTYLNWLQQKPGQPPKRLISLVSKLDSGVPD | |
| | RFSGSGSGTDFTLTISSLQAEDVAVYYCWQGTHF | |
| | PGTFGGGTKVEIK | |
| CD8 hinge and | TTTPAPRPPTPAPTIASQPLSLRPEACRPAAGGAV | 124 |
| transmembrane | HTRGLDFACDIYIWAPLAGTCGVLLLSLVITLYC | |
| 4-1BB intracellular | KRGRKKLLYIFKQPFMRPVQTTQEEDGCSCRFPE | 125 |
| signaling | EEEGGCEL | |
| CD3z intracellular | RVKFSRSADAPAYQQGQNQLYNELNLGRREEYD | 126 |
| signaling | VLDKRRGRDPEMGGKPRRKNPQEGLYNELQKD | |
| | KMAEAYSEIGMKGERRRGKGHDGLYQGLSTAT | |
| | KDTYDALHMQALPPR | |
| BFP (blue fluorescent | MSELIKENMHMKLYMEGTVDNHHFKCTSEGEG | 127 |
| protein) | KPYEGTQTMRIKVVEGGPLPFAFDILATSFLYGS | |
| | KTFINHTQGIPDFFKQSFPEGFTWERVTTYEDGG | |
| | VLTATQDTSLQDGCLIYNVKIRGVNFTSNGPVM | |
| | QKKTLGWEAFTETLYPADGGLEGRNDMALKLV | |
| | GGSHLIANIKTTYRSKKPAKNLKMPGVYYVDYR | |
| | LERIKEANNETYVEQHEVAVARYCDLPSKLGHK | |
| | LN | |
| P2A | GSGATNFSLLKQAGDVEENPGP | 128 |

In some embodiments, the CAR-immune cell (e.g., CAR-T cell) of the disclosure comprises a polynucleotide encoding a suicide polypeptide, such as for example RQR8. See, e.g., WO2013153391A, which is hereby incorporated by reference in its entirety. In some embodiments, a suicide polypeptide is expressed on the surface of the cell. In some embodiments, a suicide polypeptide is included in the CAR construct. In some embodiments, a suicide polypeptide is not part of the CAR construct.

In some embodiments, the extracellular domain of any one of CARs disclosed herein may comprise one or more epitopes specific for (specifically recognized by) a monoclonal antibody. These epitopes are also referred to herein as mAb-specific epitopes. Exemplary mAb-specific epitopes are disclosed in International Patent Publication No. WO 2016/120216, which is incorporated herein in its entirety. In these embodiments, the extracellular domain of the CARs comprise antigen binding domains that specifically bind to a target of interest and one or more epitopes that bind to one or more monoclonal antibodies (mAbs). CARs comprising the mAb-specific epitopes can be single-chain or multi-chain.

The inclusion of epitopes specific for monoclonal antibodies in the extracellular domain of the CARs described herein allows sorting and depletion of engineered immune cells expressing the CARs. In some embodiments, allowing for depletion provides a safety switch in case of deleterious effects, e.g., upon administration to a subject.

Methods of preparing immune cells for use in immunotherapy are also provided herein. In some embodiments, the methods comprise introducing a PD-1 chimeric cytokine receptor and a CAR into immune cells, and expanding the cells. In some embodiments, the invention relates to a method of engineering an immune cell comprising: providing a cell and expressing a PD-1 chimeric cytokine receptor, and expressing at the surface of the cell at least one CAR. In some embodiments, the method comprises: transfecting the cell with at least one polynucleotide encoding a PD-1 chimeric cytokine receptor, and at least one polynucleotide encoding a CAR, and expressing the polynucleotides in the cell. In some embodiments, the method comprises: transfecting the cell with at least one polynucleotide encoding a PD-1 chimeric cytokine receptor, at least one polynucleotide encoding a CAR, and expressing the polynucleotides in the cell.

In some embodiments, the polynucleotides encoding the PD-1 chimeric cytokine receptor and CAR are present in one or more expression vectors for stable expression in the cells. In some embodiments, the polynucleotides are present in viral vectors for stable expression in the cells. In some embodiments, the viral vectors may be for example, lentiviral vectors or adenoviral vectors.

In some embodiments, polynucleotides encoding polypeptides according to the present disclosure can be mRNA which is introduced directly into the cells, for example by electroporation. In some embodiments, CytoPulse electroporation technology, such as PulseAgile, can be used to transiently permeabilize living cells for delivery of material into the cells (e.g. U.S. Pat. No. 6,078,490; PCT/US2011/000827; and PCT/US2004/005237). Parameters can be modified in order to determine conditions for high transfection efficiency with minimal mortality.

Also provided herein are methods of transfecting an immune cell, e.g a T-cell. In some embodiments, the method comprises: contacting a T-cell with RNA and applying to the T-cell an agile pulse sequence. In some embodiments, a method of transfecting an immune cell (e.g. T-cell) comprising contacting the immune cell with RNA and applying to the cell an agile pulse sequence.

In some embodiments, the method can further comprise a step of genetically modifying a cell by inactivating at least one gene expressing, for example without limitation, a component of the TCR, a target for an immunosuppressive agent, an HLA gene, and/or an immune checkpoint protein such as, for example, PDCD1 or CTLA-4. By inactivating a gene it is intended that the gene of interest is not expressed in a functional protein form. In some embodiments, the gene to be inactivated is selected from the group consisting of, for example without limitation, TCRα, TCRβ, CD52, GR, deoxycytidine kinase (DCK), PD-1, and CTLA-4. In some embodiments the method comprises inactivating one or more genes by introducing into the cells a rare-cutting endonuclease able to selectively inactivate a gene by selective DNA cleavage. In some embodiments the rare-cutting endonuclease can be, for example, a transcription activator-like effector nuclease (TALE-nuclease) or CRISPR-based endonuclease (e.g Cas-9 or Cas12a).

In another aspect, a step of genetically modifying cells can comprise: modifying immune cells (e.g. T-cells) by inactivating at least one gene expressing a target for an immunosuppressive agent, and; expanding the cells, optionally in presence of the immunosuppressive agent.

In some embodiments, the engineered immune cells (e.g. T-cells) provided herein exhibit improved cytotoxicity, increased expansion, and/or increased levels of memory phenotype markers upon contact with a PD-L1 or PD-L2 ligand or PD-1 antibody that binds to the ectodomain of the PD-1 chimeric cytokine receptor relative to engineered immune cells that do not express the PD-1 chimeric cytokine receptor.

In some embodiments, the engineered immune cells (e.g. T-cells) provided herein exhibit (i) increased in vivo persistence, (ii) increased STAT activation, (iii) increased cytotoxicity, (iv) increased levels of memory phenotype markers, (v) increased expansion (proliferation), or combinations of these functional features, upon contact with a PD-L1 or PD-L2 ligand or PD-1 antibody that binds to the ectodomain of the PD-1 chimeric cytokine receptor relative to engineered immune cells that do not express the PD-1 chimeric cytokine receptor. In some embodiments, the improvement in the one or more functional features described herein is dose-dependent, i.e., the functional activity of the immune cell comprising the PD-1 chimeric cytokine receptors increases upon contact with increasing doses of the PD-L1/PD-L2/PD-1 antibody. In some embodiments, STATs activated by the engineered immune cell comprising one or more PD-1 chimeric cytokine receptors disclosed are STAT1, STAT2, STAT3, STAT4, STAT5, STAT6, or combinations thereof. In one embodiment, memory phenotype markers increased or maintained by the immune cell comprising the inducible chimeric cytokine receptor include stem cell memory (Tscm) marker and central memory (Tcm) marker.

In some embodiments, the improvement in one or more functional features exhibited by an engineered immune cell comprising an inducible chimeric cytokine receptor provided herein is at least about 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 25 fold, 30 fold, 40 fold, 50 fold, 60 fold, 70 fold, 80 fold, 90 fold, 100 fold, 125 fold, 150 fold, 200 fold, 250 fold, 300 fold, 350 fold, 400 fold, 450 fold, or even about 10500 fold, including values and ranges therebetween, compared to an immune cell that does not express the PD-1 chimeric cytokine receptor.

In some embodiments, the improvement in one or more functional features exhibited by an engineered immune cell comprising a PD-1 chimeric cytokine receptor provided herein is at least about 10%, 20%, 25%, 30%, 35%, 40%, 45%, 50%, 55%, 60%, 70%, 75%, 80%, 90%, 100%, 125%, 150%, 200%, 250%, 300%, 350%, 400%, or even about 80% 500%, including values and ranges therebetween, compared to an engineered immune cell that does not express the PD-1 chimeric cytokine receptor.

### III. Therapeutic Methods

Provided herein are pharmaceutical compositions comprising cells bearing the chimeric cytokine receptors and CARs of the disclosure.

Engineered PD-1 chimeric cytokine receptor-bearing and CAR-bearing immune cells (e.g. T-cells) obtained by the methods described above, or cell lines derived from such engineered immune cells, can be used as a medicament. In some embodiments, such a medicament can be used for treating a disorder such as for example a viral disease, a bacterial disease, a cancer, an inflammatory disease, an immune disease, or an aging-associated disease. In some embodiments, the cancer is a solid cancer. In some embodiments the cancer is a liquid cancer. The cancer can be selected from the group consisting of gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma. In some embodiments, the subject is a previously treated adult subject with locally advanced or metastatic melanoma, squamous cell head and neck cancer (SCHNC), ovarian carcinoma, sarcoma, or relapsed or refractory classic Hodgkin's Lymphoma (cHL).

In some embodiments, engineered immune cells, or cell line derived from the engineered immune cells, can be used in the manufacture of a medicament for treatment of a disorder in a subject in need thereof. In some embodiments, the disorder can be, for example, a cancer, an autoimmune disorder, or an infection.

Also provided herein are methods for treating subjects in need of such treatment.

As used herein, the term “subject” refers to any vertebrate including, without limitation, humans and other primates (e.g., chimpanzees, cynomolgous monkeys, and other apes and monkey species), farm animals (e.g., cattle, sheep, pigs, goats and horses), domestic mammals (e.g., dogs and cats), laboratory animals (e.g., rabbits, rodents such as mice, rats, and guinea pigs), and birds (e.g., domestic, wild and game birds such as chickens, turkeys and other gallinaceous birds, ducks, geese, and the like). In some embodiments, the subject is a mammal. In exemplary embodiments, the subject is a human.

In some embodiments the method comprises providing immune cells of the disclosure, bearing the PD-1 chimeric cytokine receptors and CARs described herein to a subject in need thereof.

In some embodiments, PD-1 chimeric cytokine receptor and CAR-bearing T-cells of the invention can undergo robust in vivo T-cell expansion and can persist for an extended amount of time.

Methods of treatment of the invention can be ameliorating, curative or prophylactic. The method of the invention may be either part of an autologous immunotherapy or part of an allogenic immunotherapy treatment.

In another aspect, the invention provides a method of inhibiting tumor growth or progression in a subject who has a tumor, comprising administering to the subject an effective amount of PD-1 chimeric cytokine receptor-expressing and CAR-expressing immune cells as described herein. In another aspect, the invention provides a method of inhibiting or preventing metastasis of cancer cells in a subject, comprising administering to the subject in need thereof an effective amount of engineered immune cells as described herein. In another aspect, the invention provides a method of inducing tumor regression in a subject who has a tumor, comprising administering to the subject an effective amount of engineered immune cells as described herein.

In some embodiments, the engineered T-cells herein can be administered parenterally in a subject.

Also provided is the use of any of the engineered T-cells provided herein in the manufacture of a medicament for the treatment of cancer or for inhibiting tumor growth or progression in a subject in need thereof.

In some embodiments, treatment can be administrated into subjects undergoing an immunosuppressive treatment. Indeed, the invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the subject. The administration of the cells or population of cells according to the invention may be carried out in any convenient manner, including by aerosol inhalation, injection, ingestion, transfusion, implantation or transplantation. The compositions described herein may be administered to a subject subcutaneously, intradermally, intratumorally, intranodally, intramedullary, intramuscularly, by intravenous or intralymphatic injection, or intraperitoneally. Cells bearing the CARs of the disclosure or the pharmaceutical compositions thereof may be administered via one or more of the following routes of administration: intravenous, intraocular, intravitreal, intramuscular, subcutaneous, topical, oral, transdermal, intraperitoneal, intraorbital, by implantation, by inhalation, intrathecal, intraventricular, via the ear, or intranasal.

In some embodiments the administration of the cells or population of cells (bearing the chimeric cytokine receptors and CARs of the disclosure) can comprise administration of, for example, about 10⁴ to about 10⁹ cells per kg body weight including all integer values of cell numbers within those ranges. In some embodiments the administration of the cells or population of cells can comprise administration of about 10⁴ to 10⁵ cells per kg body weight, 10⁵ to 10⁶ cells per kg body weight, 10⁶ to 10⁷ cells per kg body weight, 10⁷ to 10⁸ cells per kg body weight, or 10⁸ to 10⁹ cells per kg body weight. The cells or population of cells can be administrated in one or more doses. In some embodiments, said effective amount of cells can be administrated as a single dose. In some embodiments, said effective amount of cells can be administrated as more than one dose over a period time. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection within a tumor.

The methods can further comprise administering one or more agents to a subject prior to administering the engineered immune cells bearing a CAR and a PD-1 chimeric cytokine receptor provided herein. In certain embodiments, the agent is a lymphodepleting (preconditioning) regimen. For example, methods of lymphodepleting a subject in need of such therapy comprise administering to the subject specified beneficial doses of cyclophosphamide (between 200 mg/m²/day and 2000 mg/m²/day, about 100 mg/m²/day and about 2000 mg/m²/day; e.g., about 100 mg/m²/day, about 200 mg/m²/day, about 300 mg/m²/day, about 400 mg/m²/day, about 500 mg/m²/day, about 600 mg/m²/day, about 700 mg/m²/day, about 800 mg/m²/day, about 900 mg/m²/day, about 1000 mg/m²/day, about 1500 mg/m²/day or about 2000 mg/m²/day) and specified doses of fludarabine (between 20 mg/m²/day and 900 mg/m²/day, between about 10 mg/m²/day and about 900 mg/m²/day; e.g., about 10 mg/m²/day, about 20 mg/m²/day, about 30 mg/m²/day, about 40 mg/m²/day, about 40 mg/m²/day, about 50 mg/m²/day, about 60 mg/m²/day, about 70 mg/m²/day, about 80 mg/m²/day, about 90 mg/m²/day, about 100 mg/m²/day, about 500 mg/m²/day or about 900 mg/m²/day). An exemplary dosing regimen involves treating a subject comprising administering daily to the patient about 300 mg/m²/day of cyclophosphamide in combination or before or after administering about 30 mg/m²/day of fludarabine for three days prior to administration of a therapeutically effective amount of engineered immune cells to the patient.

In some embodiments, notably in the case when the engineered cells provided herein have been gene edited to eliminate or minimize surface expression of CD52, lymphodepletion further comprises administration of an anti-CD52 antibody, such as alemtuzumab. In some embodiments, the CD52 antibody is administered at a dose of about 1-20 mg/day IV, e.g., about 13 mg/day IV for 1, 2, 3 or more days. The antibody can be administered in combination with, before, or after administration of other elements of a lymphodepletion regime (e.g., cyclophosphamide and/or fludarabine).

In certain embodiments, compositions comprising CAR-expressing immune effector cells disclosed herein may be administered in conjunction with any number of chemotherapeutic agents.

### IV. Kits and Articles of Manufacture

The present disclosure provides kits comprising any one or more of the PD-1 chimeric cytokine receptors and PD-1 chimeric cytokine receptors-bearing cells described herein, and pharmaceutical compositions thereof. The present disclosure also provides articles of manufacture comprising any one or more of the PD-1 chimeric cytokine receptors and PD-1 chimeric cytokine receptors-bearing CAR-I cells described herein, pharmaceutical compositions thereof, and kits described herein.

The following examples are included for illustrative purposes and are not intend to limit the scope of the disclosure.

All patent and non-patent documents referenced throughout this disclosure are incorporated by reference herein in their entirety for all purposes.

### EXAMPLES

In referring to the chimeric cytokine receptors of the disclosure it is noted that certain figures refer to a “switch.” As used herein, a “switch” is used interchangeably with a “chimeric cytokine receptor”.

The following notations are used throughout the drawings and examples.
FKBP switch=a FKBP chimeric cytokine receptor
WT PD1 switch=a chimeric cytokine receptor with a wild type PD-1 ectodomain
2×PD1 switch=a chimeric cytokine receptor with 2 PD-1 ectodomains in tandem
3×PD1 switch=a chimeric cytokine receptor with 3 PD-1 ectodomains in tandem
HA PD1 switch=a chimeric cytokine receptor with a high affinity PD-1 ectodomain
HA PD1 DN=a chimeric cytokine receptor with a dominant negative high affinity PD-1 domain

### Example 1: Coupling of PD-1 Engagement with Cytokine Signaling

To couple simultaneous PD-1 engagement with cytokine signaling, a PD-1 chimeric cytokine receptor was constructed, composed of the following modules: (i) the wild type (WT) PD-1 ectodomain (21-207), (ii) a transmembrane domain with an intracellular portion having a JAK2-activating domain and (iii) STAT-recruiting domains comprising Stat-recruiting (Stat-activating) domains from cytokine receptor tails (cytotails). With this design, it was determined whether clustering or dimerization of the PD-1 chimeric cytokine receptor, in response to PD-1 ligands or anti-PD-1 antibodies, respectively, could activate the JAK-Stat pathway and mimic cytokine signaling. In order to minimize the transgene payload in the lentiviral vector, JAK2-activating domains derived from natural cytokine receptors known to signal as homodimers, such as TpoR (TPOR/MPLR(478-582)), GCSFR (GCSFR (614-710)) and GP130 (GP130 (609-700)), were selected for initial evaluation.

Two Stat-activating cytoplasmic domains derived from IL7R (316-459) and IL12Rb (775-825) were linked in tandem mimic signaling in response to IL7 and IL12, respectively. To demonstrate the utility of the PD-1 chimeric cytokine receptor in the context of CAR-T-cells, the PD-1 chimeric cytokine receptor was cloned into a lentiviral vector encoding a second generation EGFRvIII-specific CAR (2173scFv; described in Sci Transl Med. 2015 Feb. 18; 7(275): 275ra22.); furthermore, to permit stoichiometric co-expression of the PD-1 chimeric cytokine receptor and the CAR, both genes were linked via a P2A peptide linker. To facilitate the detection of transduced cells, a v5 epitope tag (KPIPNPLLGLDST (SEQ ID NO: 167)) was inserted between the scFv and CD8 hinge domain of the CAR.

A HEK293T-cell reporter assay was used to test the inducibility and magnitude of cytokine signaling. Briefly, 20,000 HEK293T-cells were plated into each well of a poly-L-lysine-coated 96-well flat-bottom plate and allowed to adhere overnight. A PD-1 chimeric cytokine receptor (2.5 ng), a Stat response element that drives Firefly Luciferase (100 ng; Promega) and Renilla Luciferase control reporter vector (1 ng; Promega) were mixed in a final volume of 5 uL in Opti-MEM (Gibco) (“DNA mix”). As a positive control, a FKBP chimeric cytokine receptor was used, in which the WT PD-1 ectodomain was replaced with FKBP12 (F36V), so that cytokine signaling could be inducible by the small molecule, AP1903. 0.3 uL Lipofectamine 2000 (Invitrogen) in 5 uL Opti-MEM was incubated at room temperature for 5 minutes and then added to the DNA mix. The mixture was incubated at room temperature for 20 minutes and the total volume of 10 uL was added to each well containing HEK-293T. 24 hours after transfection, cells were either left untreated, or treated with the indicated concentrations of anti-PD-1 (Nivolumab; Selleck Chemical), recombinant human PD-L1-Fc (Biolegend), anti-Human IgG Fc gamma secondary antibody (Thermo Fisher), or AP1903 (Apex Bio). As negative controls, hIgG4 and hIgG1 isotype controls were added in place of anti-PD-1 and PD-L1-Fc, respectively. All treatments were diluted in serum-free media. 24 hours after treatment, Stat reporter activity was evaluated using the Dual-Glo Luciferase Assay System (Promega). Fold induction of Stat5 reporter activity was normalized to that of HEK293T-cells transfected with all vectors except for the chimeric cytokine receptor and that were left untreated.

FIGS. 1A-C show schematics of the various embodiments of the PD-1 chimeric cytokine receptor.

FIG. 1B shows a schematic of the PD-1 chimeric cytokine receptor CAR vector. FIG. 1D shows a schematic of the PD-1 chimeric cytokine receptor CAR vector, and the positive control FKBP chimeric cytokine receptor CAR vector.

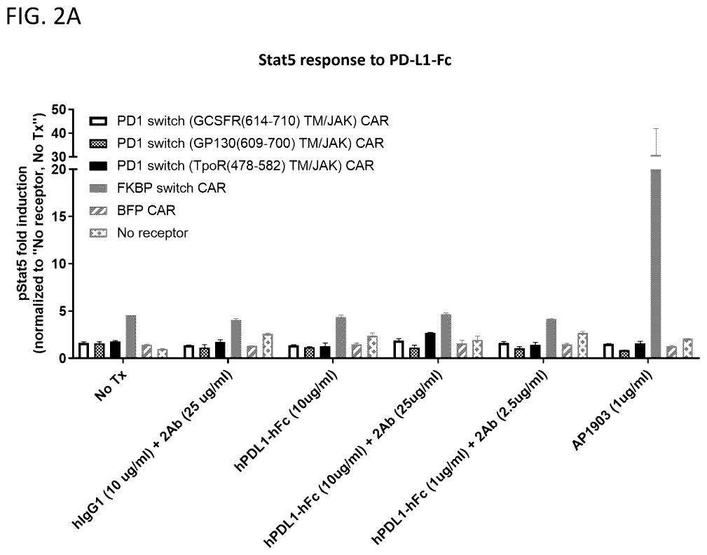

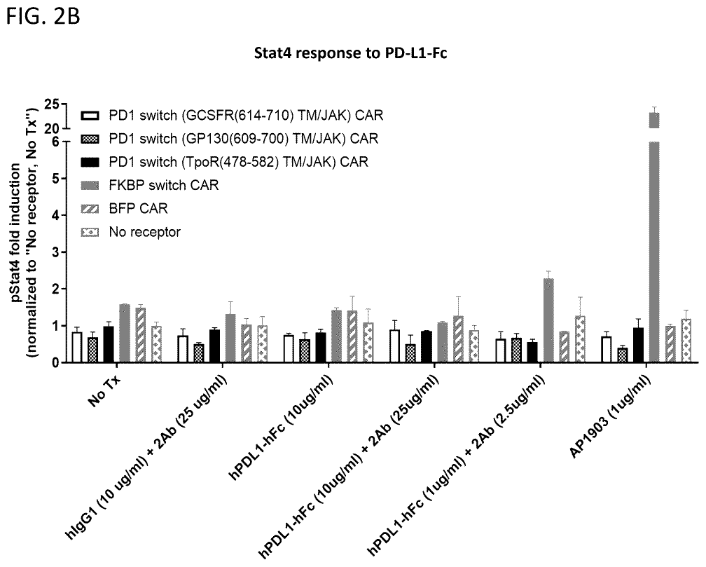

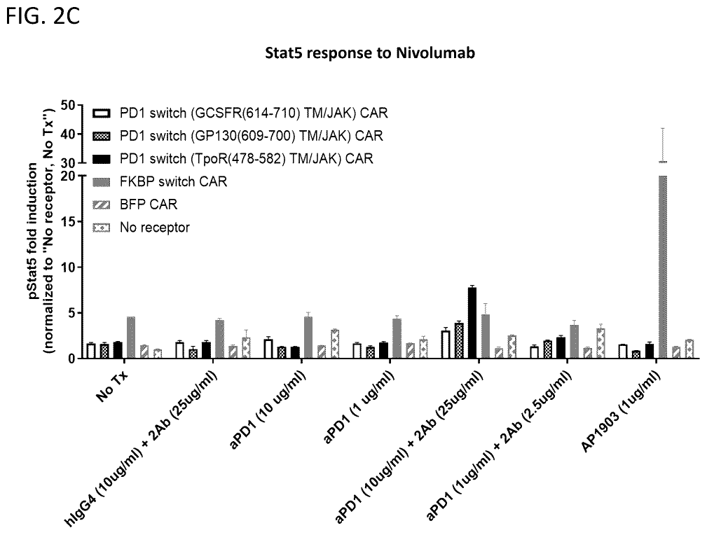

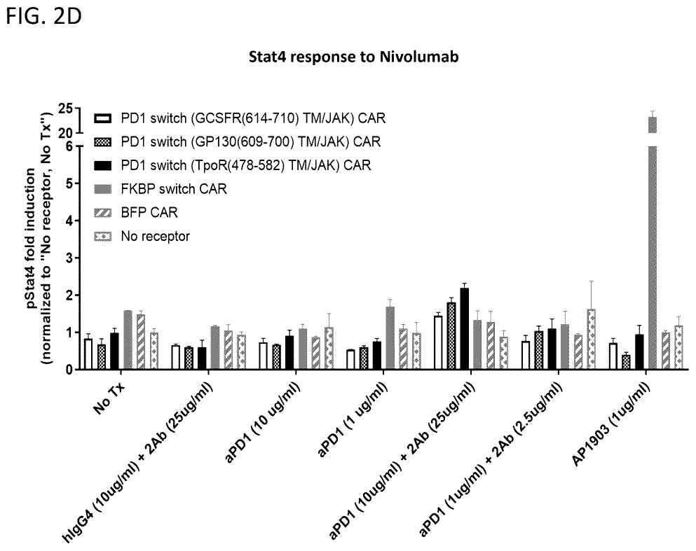

FIGS. 2A-D show Stat reporter activity in response to PD-L1-Fc (FIGS. 2A-B) and anti-PD-1 (FIGS. 2C-D). FIGS. 2A-B show that as compared to their untreated counterparts, PD-1 chimeric cytokine receptors induced weak Stat reporter activity in response to high concentrations of crosslinked PD-L1 (i.e. hPD-L1-Fc (10 ug/ml)+2Ab (25 ug/ml)). FIGS. 2C-D show that as compared to their untreated counterparts, PD-1 chimeric cytokine receptors induced weak Stat reporter activity in response to high concentrations of crosslinked anti-PD-1 (i.e. aPD-1 (10 ug/ml)+2Ab (25 ug/ml)), with the TpoR (478-582) TM/JAK variant showing the best response. Although the FKBP chimeric cytokine receptor positive control showed basal activity in the absence of AP1903, it induced high Stat reporter activity in response to AP1903.

Similar experiments were then carried out in the context of primary human CAR-T-cells. To make lentivirus encoding PD-1 chimeric cytokine receptor CARs, HEK293T-cells were plated at 0.45 million cells per mL in 2 mL of DMEM (Gibco) supplemented with 10% FBS (Hyclone) per well of a 6-well plate on Day −1. On Day 0, the lentivirus was prepared by mixing together lentiviral packaging vectors 1.5 ug psPAX2, 0.5 ug pMD2G, and 0.5 ug of the appropriate transfer CAR vector in 250 uL Opti-MEM (Gibco) per well of the 6-well plate (“DNA mix”). 10 uL Lipofectamine 2000 (Invitrogen) in 250 uL Opti-MEM was incubated at room temperature for 5 minutes and then added to the DNA mix. The mixture was incubated at room temperature for 20 minutes and the total volume of 500 uL was slowly added to the sides of the wells containing HEK293T. On Day 0, purified T-cells were activated in X-Vivo-15 medium (Lonza) supplemented with 100 IU/mL human IL-2 (Miltenyi Biotec), 10% FBS (Hyclone), and human T TransAct (Miltenyi Biotec, Cat #130-111-160, 1:100 dilution) in a Grex-24 plate (Wilson Wolf, cat #80192M). On Day 1, the media from each well of HEK293T-cells in the 6-well plate was replaced with 2 mL per well of T-cell transduction media, i.e., X-Vivo-15 supplemented with 10% FBS. On Day 2, T-cells were resuspended at 0.5 million cells per mL in 1 mL of T-cell transduction media per well of a Grex-24 plate. The lentiviral supernatants from HEK293T-cells were harvested and passed through a 0.45 micron filter (EMD Millipore) to remove cell debris, and then added to the T-cells along with 100 IU/mL human IL-2. On Day 5, 4.5 mL of T-cell expansion media, i.e., X-Vivo-15 supplemented with 5% human AB serum (Gemini Bio) and 100 IU/mL human IL-2 was added to each well of a Grex-24 plate. Cells were expanded into larger G-Rex vessels (Wilson Wolf) as needed using T-cell expansion media. On Day 13 or 14, transduction efficiency and PD-1 chimeric cytokine receptor expression was determined by detecting the percentage of T-cells that bound a FITC-conjugated v5 tag monoclonal antibody (Thermo Fisher) and a PE/Cy7-conjugated anti-human PD-1 (Biolegend) using flow cytometry. On Day 14 or 15, the CAR-T-cell products were cryopreserved and thawed as needed for further assays.

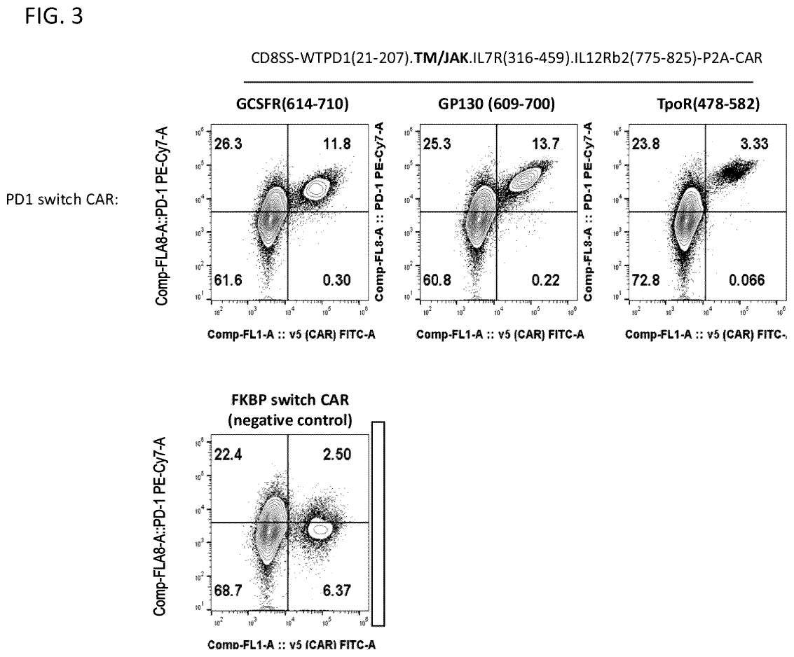

FIG. 3 shows CAR and PD-1 chimeric cytokine receptor expression on the Day 14 CAR-T-cell product. As compared to FKBP chimeric cytokine receptor CAR-T-cells on which little to no PD-1 was detected, high levels of PD-1 was detected on PD-1 chimeric cytokine receptor CAR-T-cells, indicating that the CAR and PD-1 chimeric cytokine receptor were coexpressed.

To determine the inducibility and magnitude of cytokine signaling in PD-1 chimeric cytokine receptor CAR-T-cells, the thawed CAR-T-cell product was serum starved in 100 uL serum-free RPMI (Corning) for 4 hours in humidified incubator at 37° C. with 5% CO₂, then treated with anti-PD-1 (10 ug/mL Nivolumab; Selleck Chemical), recombinant human PD-L1-Fc (10 ug/mL; Biolegend), anti-Human IgG Fc gamma secondary antibody (25 ug/mL; Thermo Fisher) for 1 hour. 40 minutes into the treatment, an antibody cocktail comprising BUV395-conjugated anti-human CD3 (Biolegend) and FITC-conjugated v5 tag monoclonal antibody (Thermo Fisher) were added to the cells and allowed to incubate for the final 20 minutes. After 1 hour of treatment, cells were fixed by the addition of 35 uL of 16% paraformaldehyde was added to each 100 uL sample and allowed to incubate for 15 minutes at 37° C. Cells were then washed three times with PBS, and permeabilized in 100% cold methanol for 1 or 2 nights at −20° C. On the day of FACS analysis, cells were washed three times with PBS, Fc-blocked, and stained with AlexaFluor647-conjugated anti-mouse/human Stat5 (pY694) (BD Biosciences) diluted in PBS+1% BSA. After a 1 hour incubation at room temperature in the dark, cells were washed three times before FACS analysis.

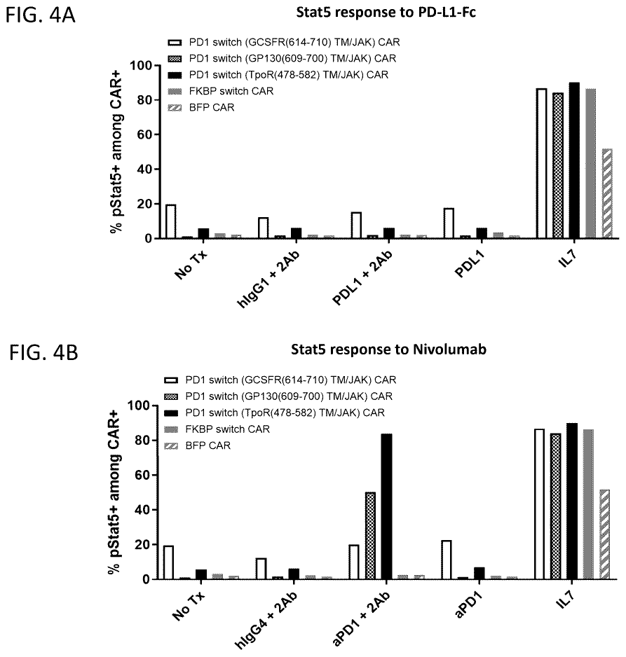

FIGS. 4A-B show phospho-Stat5 staining in human CAR-T-cells following the treatment with PD-L1-Fc (FIG. 4A) or anti-PD-1 (FIG. 4B). PD-1 chimeric cytokine receptor CAR-T-cells bearing the GCSFR (614-710) TM/JAK2-activating domain showed high basal activity in the absence of treatment, and no induction of pStat5 in the presence of treatment. PD-1 chimeric cytokine receptor CAR-T-cells bearing the GP130 (609-700) TM/JAK2-activating domain showed the lowest basal activity in the absence of treatment, no pStat5 induction in response to PD-L1 or anti-PD-1, and moderate pStat5 induction in response to crosslinked anti-PD-1. PD-1 chimeric cytokine receptor CAR-T-cells bearing the TpoR (478-582) TM/JAK2-activating domain showed the moderate basal activity in the absence of treatment, no pStat5 induction in response to PD-L1 or anti-PD-1, and strong pStat5 induction in response to crosslinked anti-PD-1.

### Example 2: Optimization of the PD-1 Chimeric Cytokine Receptor

Compared to the high affinity interaction between Nivolumab and PD-1 (<10 pM), PD-1 binds to PD-L1 and PD-L2 through a low-affinity (1-10 uM), monomeric interaction. The observation that crosslinked anti-PD-1 (Nivolumab) was able to activate the PD-1 chimeric cytokine receptor bearing the TpoR (478-582) TM/JAK2-activating domain indicating that PD-1 chimeric cytokine receptor activation could be made more efficient by enhancing its affinity or avidity of interaction with its inducer (PD-1 ligands or anti-PD-1). To increase the avidity of the PD-1 chimeric cytokine receptor, multiple WT PD-1 ectodomains were engineered in tandem. To increase the affinity of the PD-1 chimeric cytokine receptor, mutations to the PD-1 ectodomains were introduced, aimed at increasing binding affinity to its ligands (henceforth referred to as “high affinity (HA) PD-1”).

To demonstrate the utility of these PD-1 chimeric cytokine receptors in the context of CAR-T-cells, each PD-1 chimeric cytokine receptor was cloned into a lentiviral vector encoding a second generation EGFRvIII-specific CAR (2173scFv; described in Sci Transl Med. 2015 Feb. 18; 7(275): 275ra22.); furthermore, to permit stoichiometric co-expression of the PD-1 chimeric cytokine receptor and the CAR, both genes were linked via a P2A peptide linker. To facilitate the detection of transduced cells, a v5 epitope tag (KPIPNPLLGLDST (SEQ ID NO: 167)) was inserted between the scFv and CD8 hinge domain.

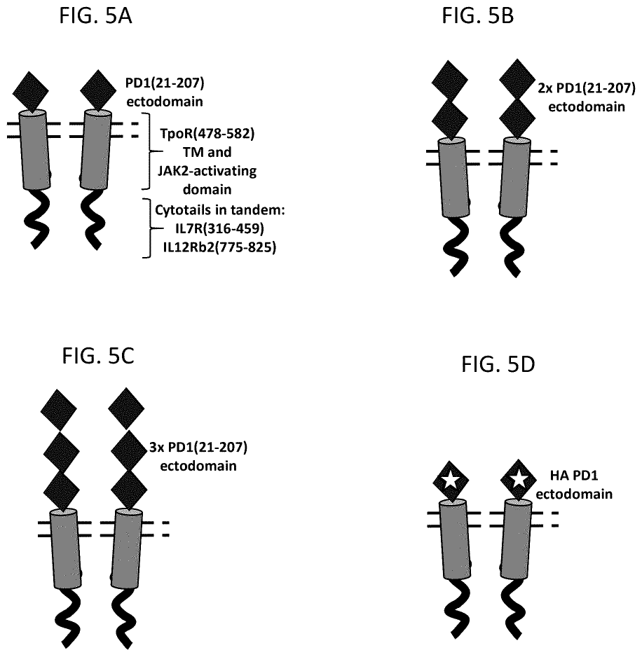

FIGS. 5A-D show a schematic of PD-1 chimeric cytokine receptors with various ectodomains engineered for enhanced avidity or affinity. FIG. 5A shows the prototypic WT PD-1 chimeric cytokine receptor bearing the TpoR (478-582) TM/JAK2-activating domain; FIG. 5B shows the 2×PD-1 chimeric cytokine receptor bearing two WT PD-1 ectodomains in tandem; FIG. 5C shows the 3×PD-1 chimeric cytokine receptor bearing three WT PD-1 ectodomains in tandem; FIG. 5D shows the high affinity (HA) PD-1 chimeric cytokine receptor with ectodomain mutations.

A HEK293T-cell reporter assay was used to test the inducibility and magnitude of cytokine signaling. Briefly, 20,000 HEK293T-cells were plated into each well of a poly-L-lysine-coated 96-well flat-bottom plate and allowed to adhere overnight. A PD-1 chimeric cytokine receptor (2.5 ng), a Stat response element that drives Firefly Luciferase (100 ng; Promega) and Renilla Luciferase control reporter vector (1 ng; Promega) were mixed in a final volume of 5 uL in Opti-MEM (Gibco) (“DNA mix”). As a positive control, a FKBP chimeric cytokine receptor was used, in which the WT PD-1 ectodomain was replaced with FKBP12 (F36V), so that cytokine signaling could be inducible by the small molecule, AP1903. 0.3 uL Lipofectamine 2000 (Invitrogen) in 5 uL Opti-MEM was incubated at room temperature for 5 minutes and then added to the DNA mix. The mixture was incubated at room temperature for 20 minutes and the total volume of 10 uL was added to each well containing HEK-293T. 24 hours after transfection, cells were either left untreated, or treated with the indicated concentrations of anti-PD-1 (Nivolumab; Selleck Chemical), recombinant human PD-L1-Fc (Biolegend), anti-Human IgG Fc gamma secondary antibody (Thermo Fisher), or AP1903 (Apex Bio). As negative controls, hIgG4 and hIgG1 isotype controls were added in place of anti-PD-1 and PD-L1-Fc, respectively. All treatments were diluted in serum-free media. 24 hours after treatment, Stat reporter activity was evaluated using the Dual-Glo Luciferase Assay System (Promega). Fold induction of Stat5 reporter activity was normalized to that of HEK293T-cells transfected with all vectors except for the chimeric cytokine receptor and that were left untreated.

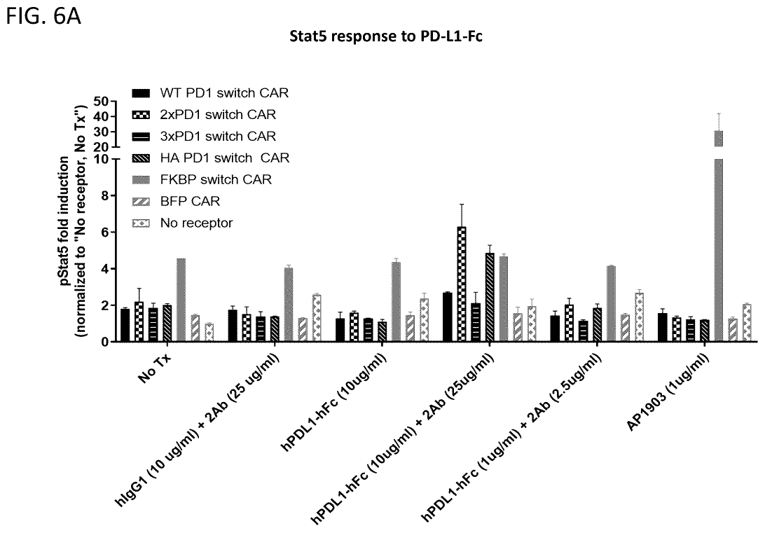

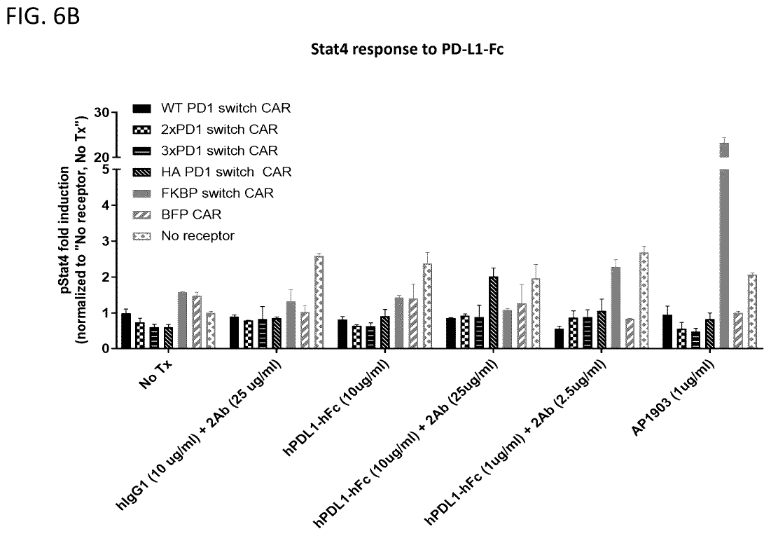

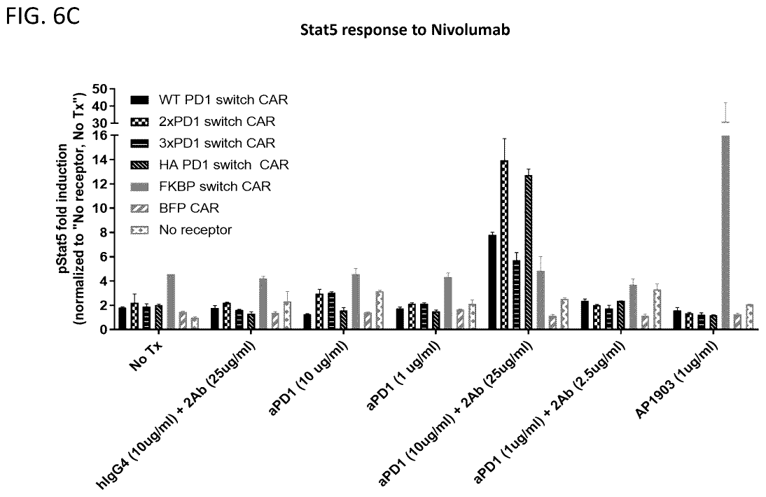

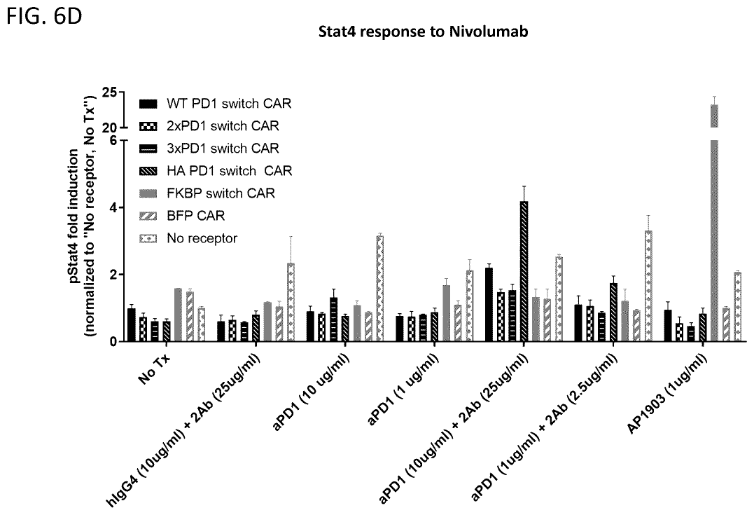

FIGS. 6A-D show the response of PD-1 chimeric cytokine receptors bearing ectodomain modifications following treatment with PD-L1-Fc (FIGS. 6A-B) and anti-PD-1 (FIGS. 6C-D). FIGS. 6A-B show that compared to its untreated counterpart and to the WT PD-1 chimeric cytokine receptor, the HA PD-1 chimeric cytokine receptor permitted enhanced Stat5 (FIG. 6A) and Stat4 (FIG. 6B) reporter activity in response to high concentrations of crosslinked PD-L1-Fc; whereas the 2×PD-1 chimeric cytokine receptor permitted enhanced Stat5, but not Stat4, reporter activity. FIGS. 6C-d show that compared to its untreated counterpart and to the WT PD-1 chimeric cytokine receptor, the HA PD-1 chimeric cytokine receptor permitted enhanced Stat5 (FIG. 6C) and Stat4 (FIG. 6D) reporter activity in response to high concentrations of crosslinked anti-PD-1; whereas the 2×PD-1 chimeric cytokine receptor permitted enhanced Stat5, but not Stat4, reporter activity. These indicated that enhancing the avidity of the ectodomain via tandem PD-1, or enhancing the affinity of the ectodomain via mutations, increase the responsiveness of the PD-1 chimeric cytokine receptor. Furthermore, the fact that the HA PD-1 chimeric cytokine receptor may be activated by anti-PD-1 indicates that the mutant HA ectodomain retains the ability to bind anti-PD-1 (i.e. Nivolumab)

Similar experiments were then carried out in the context of primary human CAR-T-cells. To make lentivirus encoding PD-1 chimeric cytokine receptor CARs, HEK293T-cells were plated at 0.45 million cells per mL in 2 mL of DMEM (Gibco) supplemented with 10% FBS (Hyclone) per well of a 6-well plate on Day −1. On Day 0, the lentivirus was prepared by mixing together lentiviral packaging vectors 1.5 ug psPAX2, 0.5 ug pMD2G, and 0.5 ug of the appropriate transfer CAR vector in 250 uL Opti-MEM (Gibco) per well of the 6-well plate (“DNA mix”). 10 uL Lipofectamine 2000 (Invitrogen) in 250 uL Opti-MEM was incubated at room temperature for 5 minutes and then added to the DNA mix. The mixture was incubated at room temperature for 20 minutes and the total volume of 500 uL was slowly added to the sides of the wells containing HEK293T. On Day 0, purified T-cells were activated in X-Vivo-15 medium (Lonza) supplemented with 100 IU/mL human IL-2 (Miltenyi Biotec), 10% FBS (Hyclone), and human T TransAct (Miltenyi Biotec, Cat #130-111-160, 1:100 dilution) in a Grex-24 plate (Wilson Wolf, cat #80192M). On Day 1, the media from each well of HEK293T-cells in the 6-well plate was replaced with 2 mL per well of T-cell transduction media, i.e., X-Vivo-15 supplemented with 10% FBS. On Day 2, T-cells were resuspended at 0.5 million cells per mL in 1 mL of T-cell transduction media per well of a Grex-24 plate. The lentiviral supernatants from HEK293T-cells were harvested and passed through a 0.45 micron filter (EMD Millipore) to remove cell debris, and then added to the T-cells along with 100 IU/mL human IL-2. On Day 5, 4.5 mL of T-cell expansion media, i.e., X-Vivo-15 supplemented with 5% human AB serum (Gemini Bio) and 100 IU/mL human IL-2 was added to each well of a Grex-24 plate. Cells were expanded into larger G-Rex vessels (Wilson Wolf) as needed using T-cell expansion media. On Day 13 or 14, transduction efficiency and PD-1 chimeric cytokine receptor expression was determined by detecting the percentage of T-cells that bound a FITC-conjugated v5 tag monoclonal antibody (Thermo Fisher) using flow cytometry. On Day 14 or 15, the CAR-T-cell products were cryopreserved and thawed as needed for further assays.

To determine if the HA PD-1 ectodomain indeed enhances binding affinity to PD-L1, while retaining interactions with clinically-approved anti-PD-1 antibodies, the ability of HA PD-1 chimeric cytokine receptor CAR-T-cells to bind Nivolumab and Pembrolizumab was tested in a cell-binding assay. Briefly, WT PD-1 chimeric cytokine receptor CAR-T-cells or HA PD-1 chimeric cytokine receptor CAR-T-cells were Fc-blocked, and then incubated with the indicated concentrations of anti-PD-1 (i.e. Nivolumab or Pembrolizumab) diluted in PBS+1% BSA. After a 20 minute incubation at 4° C., cells were washed in PBS, and incubated with a PE-conjugated anti-human IgG Fc secondary antibody (Biolegend) diluted in PBS+1% BSA. After a 20 minute incubation at 4° C., cells were washed in PBS and analyzed by flow cytometry.

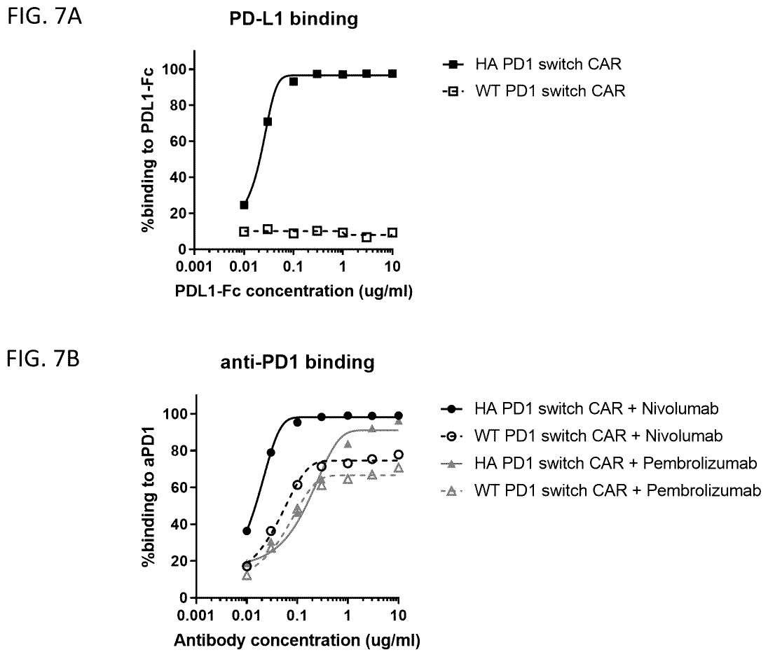

FIGS. 7A-B show results from the cell binding assay between PD-1 chimeric cytokine receptor CAR-T-cells and PD-L1-Fc (FIG. 7A) or clinically-approved anti-PD-1 antibodies (FIG. 7B). FIG. 7A shows that while the WT PD-1 chimeric cytokine receptor did not bind to PD-L1-Fc at the concentrations tested, the HA PD-1 chimeric cytokine receptor dramatically improved binding to PD-L1-Fc. FIG. 7B shows that modification to the HA PD-1 ectodomain improved maximal binding (Cₘₐₓ) to Pembrolizumab, and improved both maximal binding (Cₘₐₓ) and sensitivity (EC₅₀) to Nivolumab. The HA PD-1 ectodomain variant improves responsiveness to both PD-L1 and clinically-approved anti-PD-1 antibodies.

To analyze the inducibility and magnitude of cytokine signaling in CAR-T-cells bearing these PD-1 chimeric cytokine receptor ectodomain variants, the thawed CAR-T-cell product was serum starved in 100 uL serum-free RPMI (Corning) for 4 hours in humidified incubator at 37° C. with 5% CO₂, then treated with anti-PD-1 (10 ug/mL Nivolumab; Selleck Chemical), recombinant human PD-L1-Fc (10 ug/mL; Biolegend), anti-Human IgG Fc gamma secondary antibody (25 ug/mL; Thermo Fisher) for 1 hour. 40 minutes into the treatment, an antibody cocktail comprising BUV395-conjugated anti-human CD3 (Biolegend) and FITC-conjugated v5 tag monoclonal antibody (Thermo Fisher) were added to the cells and allowed to incubate for the final 20 minutes. After 1 hour of treatment, cells were fixed by the addition of 35 uL of 16% paraformaldehyde was added to each 100 uL sample and allowed to incubate for 15 minutes at 37° C. Cells were then washed three times with PBS, and permeabilized in 100% cold methanol for 1 or 2 nights at −20° C. On the day of FACS analysis, cells were washed three times with PBS, Fc-blocked, and stained with AlexaFluor647-conjugated anti-mouse/human Stat5 (pY694) (BD Biosciences) diluted in PBS+1% BSA. After a 1 hour incubation at room temperature in the dark, cells were washed three times before FACS analysis.

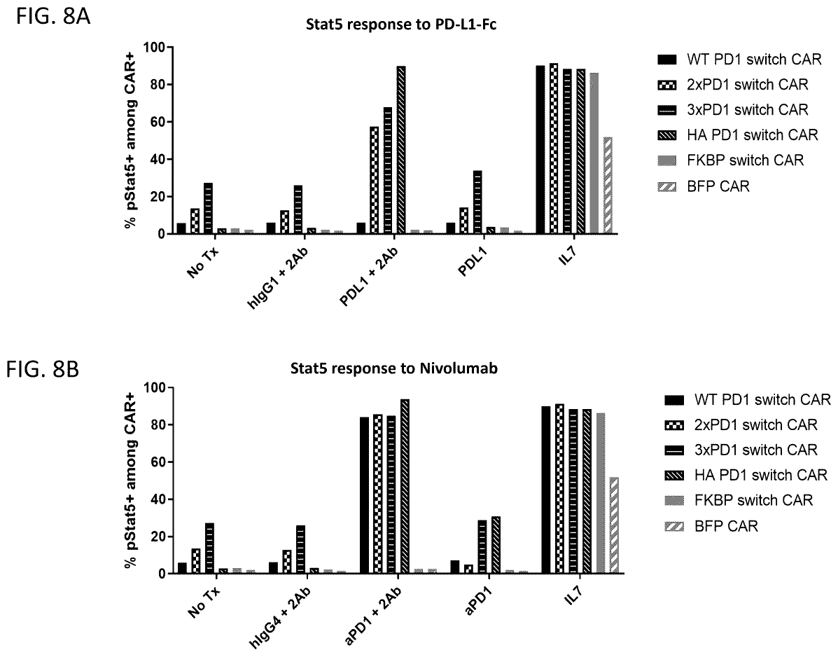

FIGS. 8A-B show the response of CAR-T-cells bearing PD-1 chimeric cytokine receptor ectodomain variants following treatment with PD-L1-Fc (FIG. 8A) or anti-PD-1 (FIG. 8B). 2×PD-1 and 3×PD-1 chimeric cytokine receptor CAR-T-cells showed high levels of basal signaling, even in the absence of PD-L1 or anti-PD-1. Compared to WT PD-1 chimeric cytokine receptor CAR-T-cells, CAR-T-cells bearing the 2×PD-1 chimeric cytokine receptor, 3×PD-1 chimeric cytokine receptor and HA PD-1 chimeric cytokine receptor showed enhanced response to crosslinked PD-L1-Fc, with the HA PD-1 chimeric cytokine receptor providing the greatest response (FIG. 8A). In response to anti-PD-1 alone, the HA PD-1 chimeric cytokine receptor was the only ectodomain variant that induced increased pStat5; neither the 2×PD-1 chimeric cytokine receptor nor the 3×PD-1 chimeric cytokine receptor induced pStat5 beyond their levels of basal activity (FIG. 8B). Based on this analysis, the HA PD-1 chimeric cytokine receptor was the ectodomain variant that provided the lowest basal activity and greatest inducibility.

To evaluate the cytotoxic activity of PD-1 chimeric cytokine receptor CAR-T-cells, the U87KO-EGFRvIII-nucGFP target cell line that endogenously expresses PD-L1 and PD-L2. U87KO-EGFRvIII (gift from Cellectis SA (Paris, France)). U87KO-EGFRvIII was derived from the parental cell line, U87MG (ATCC), by first knocking out endogenous wild type EGFR using Transcription Activator-Like Effector Nucleases (TALEN), and then stably overexpressing full-length human EGFRvIII via lentiviral transduction. To facilitate target cell imaging via the IncuCyte Live Cell Analysis Imaging System, U87KO-EGFRvIII-nucGFP target cells were derived from U87KO-EGFRvIII by a second lentiviral transduction with IncuCyte NucLight Green Lentivirus Reagent (Sartorius). PD-L1 and PD-L2 expression on U87KO-EGFRvIII-nucGFP was determined by staining with an APC-conjugated anti-human PD-L1 (Biolegend) and a PE-conjugated anti-human PD-L2 (Biolegend), followed by flow cytometry analysis. For the in vitro cytotoxicity assay, 5,000 U87KO-EGFRVIII-nucGFP target cells were seeded and allowed to attach in 96-well plates with black walls and flat clear bottom in 50 uL RPMI containing 10% FBS (Hyclone), non-essential amino acids, sodium pyruvate and 20-25 mM HEPES. EGFRVIII CAR (2173 scFv) T-cells bearing the respective PD-1 chimeric cytokine receptor ectodomain variants were thawed and added to plated target cells at an Effector:Target (E:T) ratio of 1:4. FKBP chimeric cytokine receptor CAR-T-cells that do not activate cytokine signaling in response to PD-1 ligands were used as a control. Duplicate wells were set up for each condition. The number of live target cells at each timepoint was determined by enumerating the number of live nucGFP+ target cells using the IncuCyte Live Cell Analysis Imaging System.

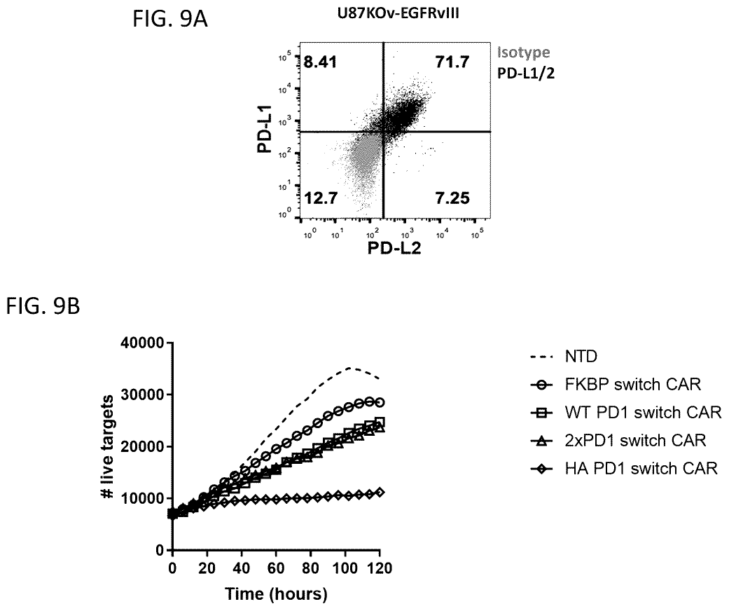

FIGS. 9A-B show the cytotoxic response of the PD-1 chimeric cytokine receptor CAR-T-cells against target cells expressing PD-1 ligands. FIG. 9A shows that U87KO-EGFRvIII-nucGFP cells express high levels of endogenous PD-L1 and PD-L2. FIG. 9B shows the results of the in vitro cytotoxic assay against U87KO-EGFRvIII-nucGFP target cells. Compared to FKBP chimeric cytokine receptor CAR-T-cells, WT PD-1 chimeric cytokine receptor CAR-T-cells showed marginally improved cytotoxic responses. While the 2×PD-1 chimeric cytokine receptor did not enhance cytotoxicity beyond that of the WT PD-1 chimeric cytokine receptor, the HA PD-1 chimeric cytokine receptor significantly enhanced CAR-T-cell cytotoxicity.

### Example 3: Optimization of the HA PD-1 Chimeric Cytokine Receptor

The HA PD-1 ectodomain variant afforded a significant enhancement over the WT PD-1 chimeric cytokine receptor. The helical transmembrane (TM) region of TpoR is critical for ligand-induced receptor signaling by controlling JAK2 activation. It was analyzed whether in a chimeric cytokine receptor where a different ectodomain (e.g. PD-1) is fused with the TpoR TM/JAK2-activating domain, the optimal structural conformation for receptor activation is perturbed. The responsiveness of the HA PD-1 chimeric cytokine receptor was attempted to be increased by modifying the TpoR TM region. To this end, HA PD-1 chimeric cytokine receptor variants with either deletions or insertions in the TpoR TM region were generated and tested in the HEK293T-cell reporter assay.

Briefly, 20,000 HEK293T-cells were plated into each well of a poly-L-lysine-coated 96-well flat-bottom plate and allowed to adhere overnight. A PD-1 chimeric cytokine receptor (2.5 ng), a Stat response element that drives Firefly Luciferase (100 ng; Promega) and Renilla Luciferase control reporter vector (1 ng; Promega) were mixed in a final volume of 5 uL in Opti-MEM (Gibco) (“DNA mix”). As a positive control, a FKBP chimeric cytokine receptor was used, in which the WT PD-1 ectodomain was replaced with FKBP12 (F36V), so that cytokine signaling could be inducible by the small molecule, AP1903. 0.3 uL Lipofectamine 2000 (Invitrogen) in 5 uL Opti-MEM was incubated at room temperature for 5 minutes and then added to the DNA mix. The mixture was incubated at room temperature for 20 minutes and the total volume of 10 uL was added to each well containing HEK-293T. 24 hours after transfection, cells were either left untreated, or treated with the indicated concentrations of anti-PD-1 (Nivolumab; Selleck Chemical), recombinant human PD-L1-Fc (Biolegend), anti-Human IgG Fc gamma secondary antibody (Thermo Fisher), or AP1903 (Apex Bio). As negative controls, hIgG4 and hIgG1 isotype controls were added in place of anti-PD-1 and PD-L1-Fc, respectively. All treatments were diluted in serum-free media. 24 hours after treatment, Stat reporter activity was evaluated using the Dual-Glo Luciferase Assay System (Promega). Fold induction of Stat5 reporter activity was normalized to that of HEK293T-cells transfected with all vectors except for the chimeric cytokine receptor and that were left untreated.

FIGS. 10A-B show the amino acid sequences for the wild type TpoR and the various TM deletion (FIG. 10A) or insertion (FIG. 10B) variants.

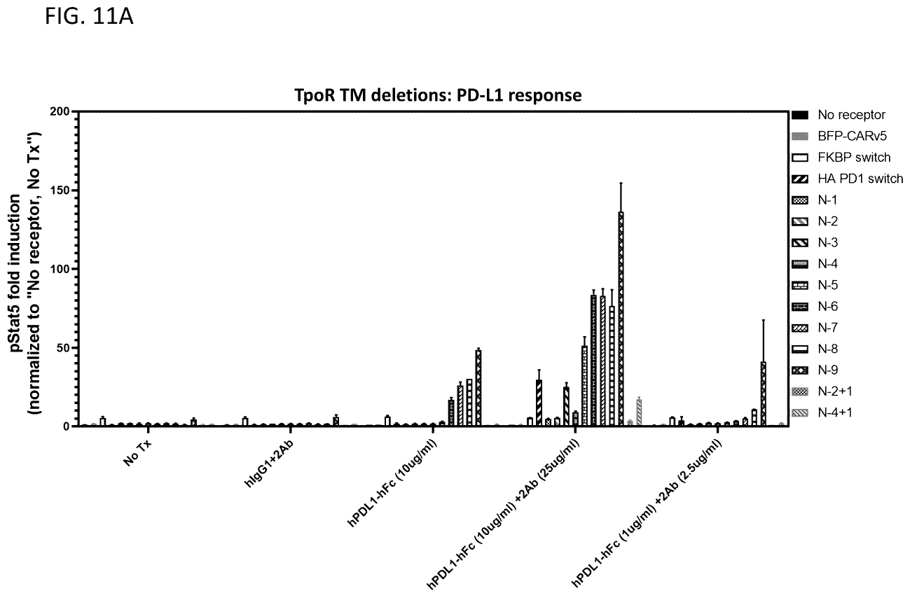

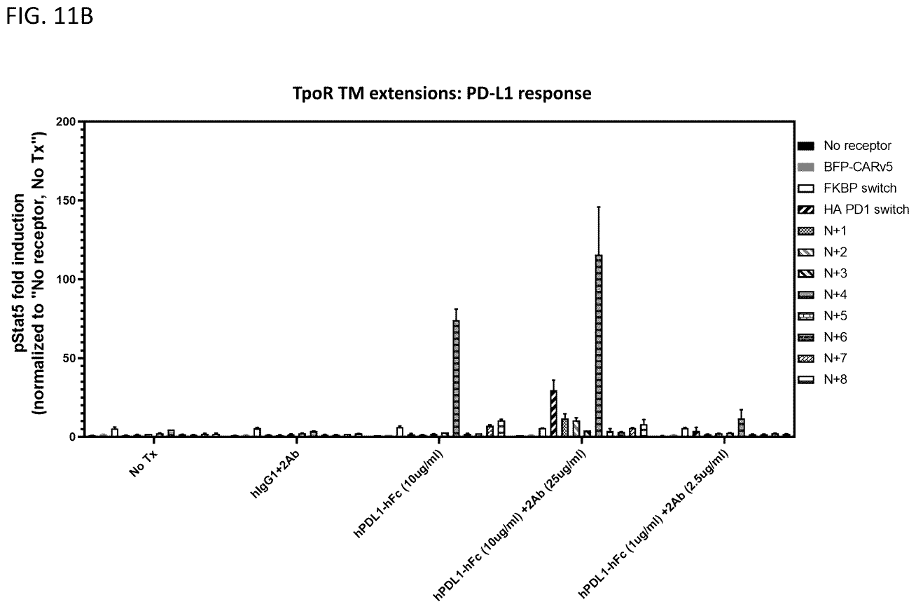

FIGS. 11A-B show the response of a HA PD-1 chimeric cytokine receptor bearing TpoR TM deletions (FIG. 11A) and extensions (FIG. 11B) following treatment with PD-L1-Fc. Unlike the HA PD-1 chimeric cytokine receptor that did not respond to PD-L1-Fc alone, the N−6, N−7, N−8, N−9 and N+4 TpoR TM variants were able to induce Stat5 activity in response to PD-L1 alone. Furthermore, compared to the HA PD-1 chimeric cytokine receptor, the N−5, N−6, N−7, N−8, N−9 and N+4 TpoR TM variants improved Stat5 activity in response to high concentrations of crosslinked PD-L1. The N−9 and N+4 TpoR TM variants retained the ability to respond to low concentrations of crosslinked PD-L1. These demonstrate that modulating the TpoR TM region enhanced the responsiveness and sensitivity of the HA PD-1 chimeric cytokine receptor to PD-1 ligands.

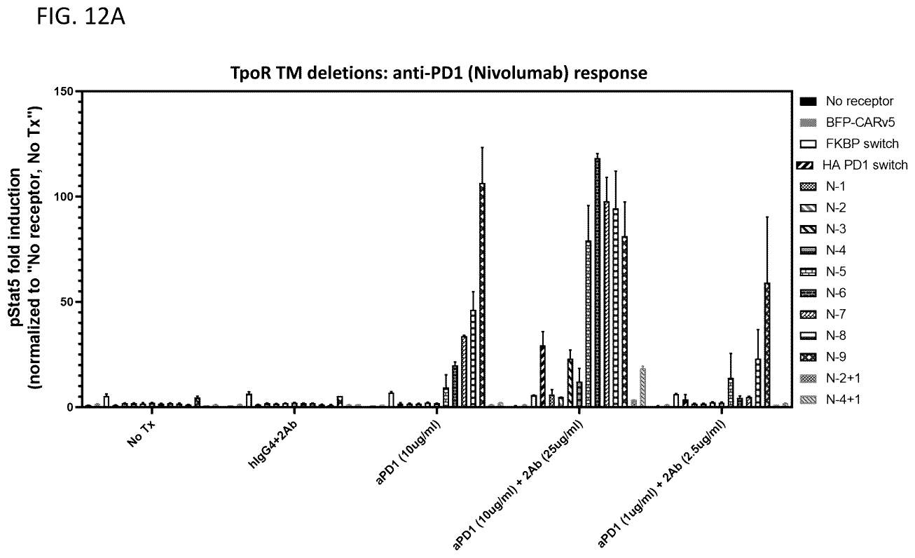

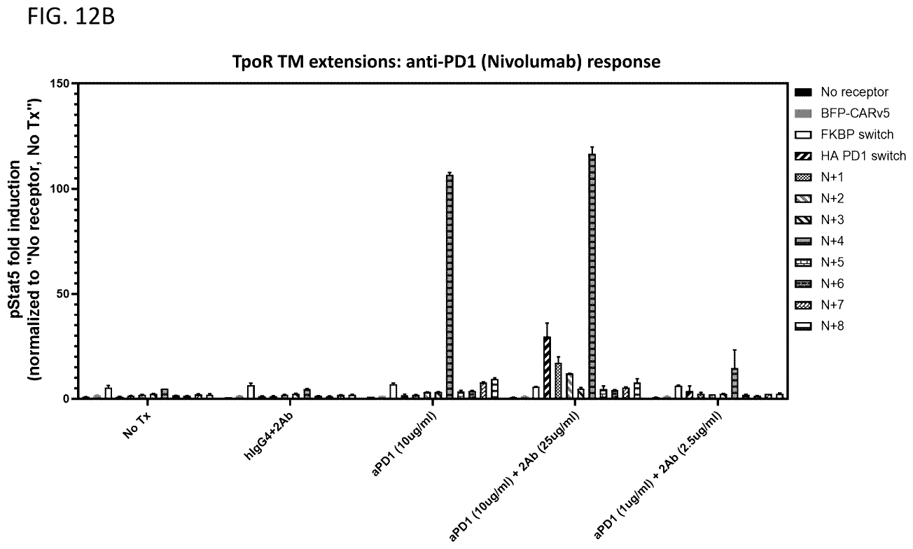

FIGS. 12A-B shows the response of HA PD-1 chimeric cytokine receptor bearing TpoR TM deletions (FIG. 12A) and extensions (FIG. 12B) following treatment with anti-PD-1 (Nivolumab). Unlike the HA PD-1 chimeric cytokine receptor that did not respond to anti-PD-1 alone, the N−5, N−6, N−7, N−8, N−9 and N+4 TpoR TM variants were able to induce Stat5 activity in response to anti-PD-1 alone, without the need for further crosslinking. These demonstrate that modulating the TpoR TM region enhanced the responsiveness and sensitivity of the HA PD-1 chimeric cytokine receptor to anti-PD-1.

### Example 4: Effect of the HA PD-1 Chimeric Cytokine Receptor on CAR-T-Cell Cytotoxic Activity

PD-1 blockade strategies, such as combination with anti-PD-1 or forced overexpression of a dominant-negative (DN) PD-1 decoy receptor, can ameliorate CAR-T-cell exhaustion. While PD-1 blockade prevents a negative signal from being transmitted into CAR-T-cells, it does not actively transmit any positive signals. It was therefore tested whether the HA PD-1 chimeric cytokine receptor, which couples PD-1 inhibition with simultaneous immune-potentiating cytokine signals, is more effective than PD-1 blockade alone. The cytotoxic activity of HA PD-1 chimeric cytokine receptor CAR-T-cells was compared against CAR-T-cells combined with Nivolumab, and against CAR-T-cells expressing a dominant-negative (DN) PD-1. (amino acid sequences in Table 9).

TABLE 9
PD1 dominant negative (DN) sequences:

| | | SEQ |
|---|---|---|
| PD1 DN | Amino acid sequence | ID NO: |
| CD8SS- | MALPVTALLLPLALLLHAARP | 129 |
| PD1(21-207) | PGWFLDSPDRPWNPPTFSPALLVVTEGDNATFTCSFSNTSESF | |
| (Dominant | VLNWYRMSPSNQTDKLAAFPEDRSQPGQDCRFRVTQLPNGR | |
| negative wild | DFHMSVVRARRNDSGTYLCGAISLAPKAQIKESLRAELRVTE | |
| type PD1) | RRAEVPTAHPSPSPRPAGQFQTLVVGVVGGLLGSLVLLVWV | |
| | LAVICSRAARGTIGARRTGQ | |
| CD8SS- | MALPVTALLLPLALLLHAARP | 130 |
| HAPD1 | PGWFLDSPDRPWNPPTFSPALLVVTEGDNATFTCSFSNTSESF | |
| (Dominant | HVIWHRESPSGQTDTLAAFPEDRSQPGQDCRFRVTQLPNGRD | |
| negative high | FHMSVVRARRNDSGTYVCGVISLAPKIQIKESLRAELRVTER | |
| affinity PD1) | RAEVPTAHPSPSPRPAGQFQTLVVGVVGGLLGSLVLLVWVL | |
| | AVICSRAARGTIGARRTGQ | |

To assess in vitro cytotoxicity, 5,000 PD-L1+/PD-L2+U87KO-EGFRvIII-nucGFP target cells were seeded and allowed to attach in 96-well plates with black walls and flat clear bottom in 50 uL RPMI containing 10% FBS (Hyclone), non-essential amino acids, sodium pyruvate and 20-25 mM HEPES. EGFRVIII CAR (2173 scFv) T-cells bearing the respective PD-1 chimeric cytokine receptors or DN PD-1 were thawed and added to plated target cells at an Effector:Target (E:T) ratio of 1:4. FKBP chimeric cytokine receptor CAR-T-cells that do not activate cytokine signaling in response to PD-1 ligands were used as a control. Where indicated, anti-PD-1 (Nivolumab) or hIgG4 isotype control antibodies were added at a concentration of 10 ug/mL. Duplicate wells were set up for each condition. The number of live target cells at each timepoint was determined by enumerating the number of live nucGFP+ target cells using the IncuCyte Live Cell Analysis Imaging System.

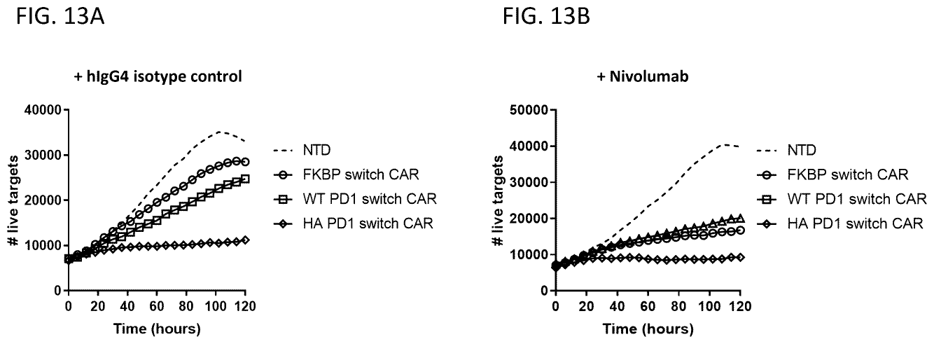

FIGS. 13A-B show the cytotoxic response of PD-1 chimeric cytokine receptor CAR-T-cells in the absence (FIG. 13A) or presence (FIG. 13B) of anti-PD-1. Although combination with anti-PD-1 did improve the activity of FKBP chimeric cytokine receptor CAR-T-cells and WT PD-1 CAR-T-cells, HA PD-1 chimeric cytokine receptor CAR-T-cells were more effective at target cell lysis. Combination with anti-PD-1 did not further improve the activity of HA PD-1 chimeric cytokine receptor CAR-T-cells, indicating that PD-1 ligands expressed on the target cells could bind to and activate the PD-1 chimeric cytokine receptor.

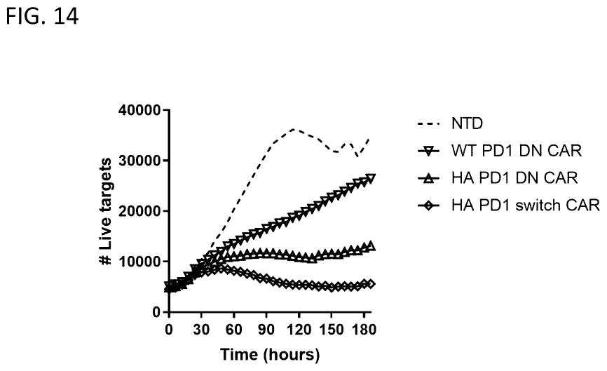

FIG. 14 shows the cytotoxic response of CAR-T-cells coexpressing either a DN WT PD-1, a DN HA PD-1 or the HA PD-1 chimeric cytokine receptor. HA PD-1 DN CAR-T-cells were more effective than WT PD-1 CAR-T-cells. Without being bound by any theory, this could be due to more effective sequestration and blockade of PD-1 ligands. However, HA PD-1 chimeric cytokine receptor CAR-T-cells were more cytotoxic than HA PD-1 DN CAR-T-cells. Without being bound by any theory, this could be due to the coupling of PD-1 blockade with simultaneous cytokine signaling.

### Example 5: PD-1 Chimeric Cytokine Receptor that Delivers Constitutive Cytokine Signals in a Ligand-Independent Fashion

The TM/JAK2-activating domain of PD1 chimeric cytokine receptors in some of the examples above is derived from TpoR, whose signaling activity can be modulated by key residues within the TpoR transmembrane (TM) domain. For instance, single point mutations within the TpoR TM domain, such as at S505 or W515, have been reported to cause constitutive TpoR signaling; on the other hand, mutating H499 in the TpoR TM domain dampens signaling from these activating mutations (Proc Natl Acad Sci USA. 2013 Feb. 12; 110(7):2540-5.; FASEB J. 2011 July; 25(7):2234-44.; J Biol Chem. 2016 Feb. 5; 291(6):2974-87.). It was analyzed whether a PD-1 chimeric cytokine receptor can be constitutively active by introducing the above-described single point mutations into the TpoR TM region of the receptor. To this end, HA PD-1 chimeric cytokine receptor variants with constitutively active single point mutations (S505 and/or W515) of the TpoR TM region and a single point mutation that reduces the activity of the TpoR TM region were generated. To mimic signaling from either IL-7 or IL-2/IL15, recruiting domains derived from the IL-7Ra and IL-2/IL15Rb receptors, respectively, were fused downstream of the TpoR TM/JAK2-activating domain. Exemplary amino acid sequences of the constructs are in Table 10.

TABLE 10
PD1 chimeric cytokine receptor sequences:

| | | SEQ |
|---|---|---|
| Receptor | Amino acid sequence | ID NO: |
| CD8SS- | MALPVTALLLPLALLLHAARP | 141 |
| HAPD1.TPOR/ | PGWFLDSPDRPWNPPTFSPALLVVTEGDNATFTCSFSNTSESF | |
| MPLR(478-582; | HVIWHRESPSGQTDTLAAFPEDRSQPGQDCRFRVTQLPNGRD | |
| H499L, S505N, | FHMSVVRARRNDSGTYVCGVISLAPKIQIKESLRAELRVTER | |
| W515K). | RAEVPTAHPSPSPRPAGQFQTLV | |
| IL7Ra(316-459) | SDPTRVETATETAWISLVTALLLVLGLNAVLGLLLLRKQFPA | |
| | HYRRLRHALWPSLPDLHRVLGQYLRDTAALSPPKATVSDTC | |
| | EEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| CD8SS- | MALPVTALLLPLALLLHAARP | 142 |
| HAPD1.TpoR | PGWFLDSPDRPWNPPTFSPALLVVTEGDNATFTCSFSNTSESF | |
| (478-582; | HVIWHRESPSGQTDTLAAFPEDRSQPGQDCRFRVTQLPNGRD | |
| S505N, W515K). | FHMSVVRARRNDSGTYVCGVISLAPKIQIKESLRAELRVTER | |
| IL2Rbsmall | RAEVPTAHPSPSPRPAGQFQTLV | |
| (393-433, 518- | SDPTRVETATETAWISLVTALHLVLGLNAVLGLLLLRKQFPA | |
| 551) | HYRRLRHALWPSLPDLHRVLGQYLRDTAALSPPKATVSDTC | |
| | EEVEPSLLEILPKSSERTPLPL | |
| | DEGVAGAPTGSSPQPLQPLSGEDDAYCTFPSRDDLLLFSPSG | |
| | QGEFRALNARLPLNTDAYLSLQELQGQDPTHLV | |
| CD8SS- | MALPVTALLLPLALLLHAARP | 143 |
| HAPDIDN | PGWFLDSPDRPWNPPTFSPALLVVTEGDNATFTCSFSNTSESF | |
| | HVIWHRESPSGQTDTLAAFPEDRSQPGQDCRFRVTQLPNGRD | |
| | FHMSVVRARRNDSGTYVCGVISLAPKIQIKESLRAELRVTER | |
| | RAEVPTAHPSPSPRPAGQFQTLVVGVVGGLLGSLVLLVWVL | |
| | AVICSRAARGTIGARRTGQ | |
| CD8SS- | MALPVTALLLPLALLLHAARP | 144 |
| HAPD1.TpoR | PGWFLDSPDRPWNPPTFSPALLVVTEGDNATFTCSFSNTSESF | |
| (478-582). | HVIWHRESPSGQTDTLAAFPEDRSQPGQDCRFRVTQLPNGRD | |
| IL7Ra(316-459) | FHMSVVRARRNDSGTYVCGVISLAPKIQIKESLRAELRVTER | |
| | RAEVPTAHPSPSPRPAGQFQTLV | |
| | SDPTRVETATETAWISLVTALHLVLGLSAVLGLLLLRWQFPA | |
| | HYRRLRHALWPSLPDLHRVLGQYLRDTAALSPPKATVSDTC | |
| | EEVEPSLLEILPKSSERTPLPL | |
| | ARDEVEGFLQDTFPQQLEESEKQRLGGDVQSPNCPSEDVVIT | |
| | PESFGRDSSLTCLAGNVSACDAPILSSSRSLDCRESGKNGPHV | |
| | YQDLLLSLGTTNSTLPPPFSLQSGILTLNPVAQGQPILTSLGSN | |
| | QEEAYVTMSSFYQNQ | |
| CD8SS- | MALPVTALLLPLALLLHAARP | 145 |
| HAPD1.TpoR | | |
| (478-582). | PGWFLDSPDRPWNPPTFSPALLVVTEGDNATFTCSFSNTSESF | |
| IL2Rbsmall | HVIWHRESPSGQTDTLAAFPEDRSQPGQDCRFRVTQLPNGRD | |
| (393- | FHMSVVRARRNDSGTYVCGVISLAPKIQIKESLRAELRVTER | |
| 433, 518-551) | RAEVPTAHPSPSPRPAGQFQTLV | |
| | SDPTRVETATETAWISLVTALHLVLGLSAVLGLLLLRWQFPA | |
| | HYRRLRHALWPSLPDLHRVLGQYLRDTAALSPPKATVSDTC | |
| | EEVEPSLLEILPKSSERTPLPL | |
| | DEGVAGAPTGSSPQPLQPLSGEDDAYCTFPSRDDLLLFSPSG | |
| | QGEFRALNARLPLNTDAYLSLQELQGQDPTHLV | |
| CD8SS- | MALPVTALLLPLALLLHAARPPGWFLDSPDRPWNPPTFSPAL | 210 |
| HAPD1.TPOR/ | LVVTEGDNATFTCSFSNTSESFHVIWHRESPSGQTDTLAAFPE | |
| MPLR(478-582; | DRSQPGQDCRFRVTQLPNGRDFHMSVVRARRNDSGTYVCG | |
| H499L, S505N, | VISLAPKIQIKESLRAELRVTERRAEVPTAHPSPSPRPAGQFQT | |
| W515K). | LVSDPTRVETATETAWISLVTALLLVLGLNAVLGLLLLRKQF | |
| IL7Ra(316-459) | PAHYRRLRHALWPSLPDLHRVLGQYLRDTAALSPPKATVSD | |
| | TCEEVEPSLLEILPKSSERTPLPLLEARDEVEGFLQDTFPQQLE | |
| | ESEKQRLGGDVQSPNCPSEDVVITPESFGRDSSLTCLAGNVS | |
| | ACDAPILSSSRSLDCRESGKNGPHVYQDLLLSLGTTNSTLPPP | |
| | FSLQSGILTLNPVAQGQPILTSLGSNQEEAYVTMSSFYQNQ | |
| CD8SS- | MALPVTALLLPLALLLHAARPPGWFLDSPDRPWNPPTFSPAL | 211 |
| HAPD1.TpoR | LVVTEGDNATFTCSFSNTSESFHVIWHRESPSGQTDTLAAFPE | |
| (478-582; | DRSQPGQDCRFRVTQLPNGRDFHMSVVRARRNDSGTYVCG | |
| S505N, W515K). | VISLAPKIQIKESLRAELRVTERRAEVPTAHPSPSPRPAGQFQT | |
| IL2Rbsmall | LVSDPTRVETATETAWISLVTALHLVLGLNAVLGLLLLRKQF | |
| (393-433, 518- | PAHYRRLRHALWPSLPDLHRVLGQYLRDTAALSPPKATVSD | |
| 551) | TCEEVEPSLLEILPKSSERTPLPLLEDEGVAGAPTGSSPQPLQP | |
| | LSGEDDAYCTFPSRDDLLLFSPSGQGEFRALNARLPLNTDAY | |
| | LSLQELQGQDPTHLV | |
| CD8SS- | MALPVTALLLPLALLLHAARPPGWFLDSPDRPWNPPTFSPAL | 212 |
| HAPD1.TpoR | LVVTEGDNATFTCSFSNTSESFHVIWHRESPSGQTDTLAAFPE | |
| (478-582). | DRSQPGQDCRFRVTQLPNGRDFHMSVVRARRNDSGTYVCG | |
| IL7Ra(316-459) | VISLAPKIQIKESLRAELRVTERRAEVPTAHPSPSPRPAGQFQT | |
| | LVSDPTRVETATETAWISLVTALHLVLGLSAVLGLLLLRWQF | |
| | PAHYRRLRHALWPSLPDLHRVLGQYLRDTAALSPPKATVSD | |
| | TCEEVEPSLLEILPKSSERTPLPLLEARDEVEGFLQDTFPQQLE | |
| | ESEKQRLGGDVQSPNCPSEDVVITPESFGRDSSLTCLAGNVS | |
| | ACDAPILSSSRSLDCRESGKNGPHVYQDLLLSLGTTNSTLPPP | |
| | FSLQSGILTLNPVAQGQPILTSLGSNQEEAYVTMSSFYQNQ | |
| CD8SS- | MALPVTALLLPLALLLHAARPPGWFLDSPDRPWNPPTFSPAL | 213 |
| HAPD1.TpoR | LVVTEGDNATFTCSFSNTSESFHVIWHRESPSGQTDTLAAFPE | |
| (478-582). | DRSQPGQDCRFRVTQLPNGRDFHMSVVRARRNDSGTYVCG | |
| IL2Rbsmall | VISLAPKIQIKESLRAELRVTERRAEVPTAHPSPSPRPAGQFQT | |
| (393- | LVSDPTRVETATETAWISLVTALHLVLGLSAVLGLLLLRWQF | |
| 433, 518-551) | PAHYRRLRHALWPSLPDLHRVLGQYLRDTAALSPPKATVSD | |
| | TCEEVEPSLLEILPKSSERTPLPLLEDEGVAGAPTGSSPQPLQP | |
| | LSGEDDAYCTFPSRDDLLLFSPSGQGEFRALNARLPLNTDAY | |
| | LSLQELQGQDPTHLV | |

A HEK293T cell reporter assay was used to test for constitutive cytokine signaling by PD1 chimeric cytokine receptors. Briefly, 20,000 HEK293T cells were plated into each well of a poly-L-lysine-coated 96-well flat-bottom plate and allowed to adhere overnight. A PD1 chimeric cytokine receptor-CAR construct (2.5 ng), a Stat5 response element that drives Firefly Luciferase (100 ng; Promega) and Renilla Luciferase control reporter vector (1 ng; Promega) were mixed in a final volume of 5 μL in Opti-MEM (Gibco) (“DNA mix”). As negative controls, cells were transfected with either a BFP CAR construct that expresses BFP instead of a PD1 chimeric cytokine receptor, or a dominant negative HA PD1 ectodomain CAR construct. As a comparison, cells were transfected with a vector encoding an inducible PD1 chimeric cytokine receptor (SEQ ID NOs: 213-214). As a positive control, cells were transfected with a vector encoding full-length human EpoR (in place of the cytokine receptor-CAR construct) so that Stat5 signaling could be induced by the addition of exogeneous recombinant human Epo. 0.3 μL Lipofectamine 2000 (Invitrogen) in 5 μL Opti-MEM was incubated at room temperature for 5 minutes and then added to the DNA mix. The mixture was incubated at room temperature for 20 minutes and the total volume of 10 μL was added to each well containing HEK-293T. 24 hours after transfection, human PD-L1 Fc, a secondary crosslinking antibody, or an isotype control antibody were added at the indicated final concentrations. 48 hours after transfection, Stat5 reporter activity was evaluated using the Dual-Glo Luciferase Assay System (Promega). Fold induction of Stat5 reporter activity was normalized to that of HEK293T cells transfected with all vectors except for the cytokine receptor and that were left untreated.

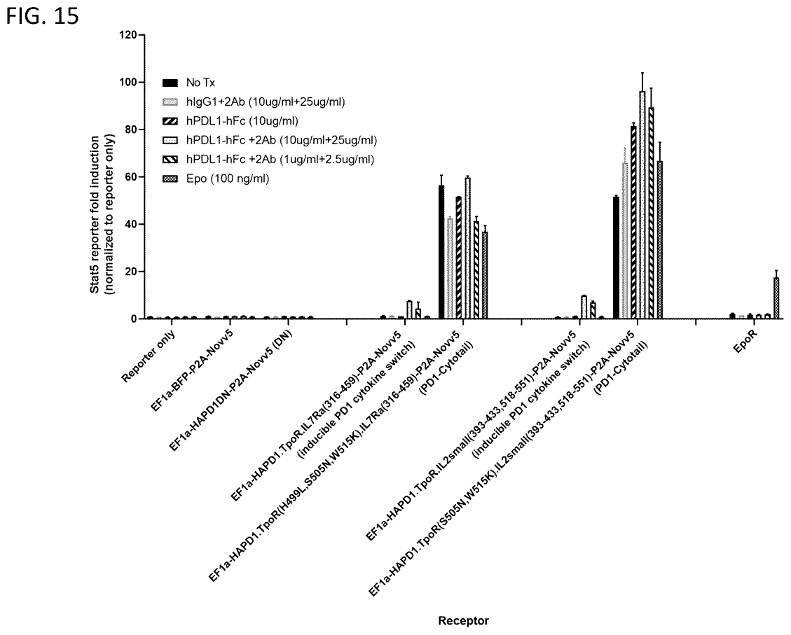

FIG. 15 shows Stat5 reporter activity in the absence and presence of PD-L1. As expected, negative controls (i.e. BFP CAR and dominant negative HA PD1 ectodomain CAR constructs) did not induce Stat5 reporter activity. The inducible PD1 chimeric cytokine receptors bearing a TM/JAK2-activating domain derived from wild type TpoR ((SEQ ID NOs: 213-214) was active in the presence of crosslinked PD-L1, but was inactive in the absence of ligand. In contrast, strong, constitutive signaling was generated from PD1 chimeric cytokine receptors comprising a TM/JAK2-activating domain derived from a TpoR transmembrane domain with constitutively active point mutations (SEQ ID NOs.: 210-211) in a ligand-independent fashion. This ligand-independence is in contrast to an inducible PD1 chimeric cytokine receptor—bearing the wild type TpoR transmembrane domain—where the presence of PD1 ligands is required for cytokine signaling to occur. The constitutive activity of the PD1 chimeric cytokine receptor comprising the TM/JAK2-activating domain derived from a TpoR transmembrane domain with constitutively active point mutations (SEQ ID NO.: 2211) was increased in the presence of PD-L1 and crosslinked PD-L1. The activity of the PD1 chimeric cytokine receptor that comprises the constitutively active TpoR TM mutations and the inhibitor mutation (H499) (SEQ ID NO.: 210) showed a slight dampening of the constitutive activity in the presence of PD-L1 and crosslinked PD-L1.

### Example 6 Increasing the Sensitivity and Responsiveness of the HA PD1 Switch Further Improves CAR T Cell Cytotoxicity

As demonstrated above, the HA PD1 switch could be engineered for increased sensitivity and responsiveness towards PD-1 ligands and anti-PD-1 by modifying the TpoR TM helix. Specifically, in the HEK293T cell reporter assay, the N−5, N−6, N−7, N−8, N−9 and N+4 TpoR TM variants enhanced STAT5 activation in response to crosslinked PD-L1 (FIG. 11) and anti-PD-1 (FIG. 12). To evaluate if this translated into improved CAR T cell cytotoxicity in the presence of PD-1 ligands and anti-PD-1, we compared the killing activity of HA PD1 switch CAR T cells bearing either the wildtype (WT) or variant TpoR TM domains.

Briefly, 5,000 PD-L1+/PD-L2+ U87KO-EGFRvIII-nucGFP target cells were seeded and allowed to attach in 96-well plates with black walls and flat clear bottom in 50 uL RPMI containing 10% FBS (Hyclone), non-essential amino acids, sodium pyruvate and 20-25 mM HEPES. EGFRVIII CAR (2173 scFv) T cells coexpressing a HA PD1 switch with the indicated TpoR TM domain were thawed and added to plated target cells at an Effector:Target (E:T) ratio of 1:4 or 1:8. As a control, CAR T cells coexpressing BFP in place of a HA PD1 switch, and that do not activate cytokine signaling in response to PD-1 ligands, were used. Where indicated, anti-PD-1 (nivolumab) or hIgG4 isotype control antibodies were added at a concentration of 10 ug/mL. Duplicate wells were set up for each condition. The number of live target cells at each timepoint was determined by enumerating the number of live nucGFP+ target cells using the IncuCyte Live Cell Analysis Imaging System.

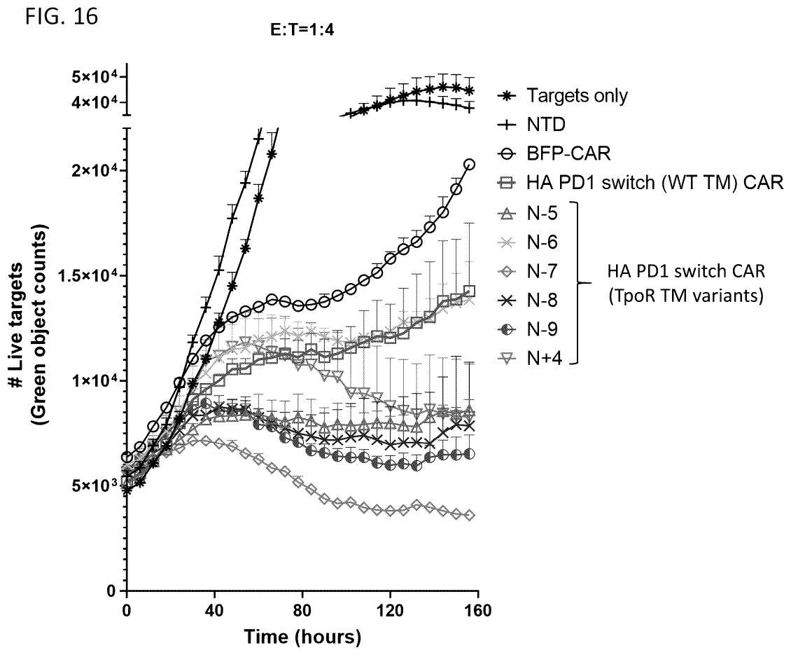

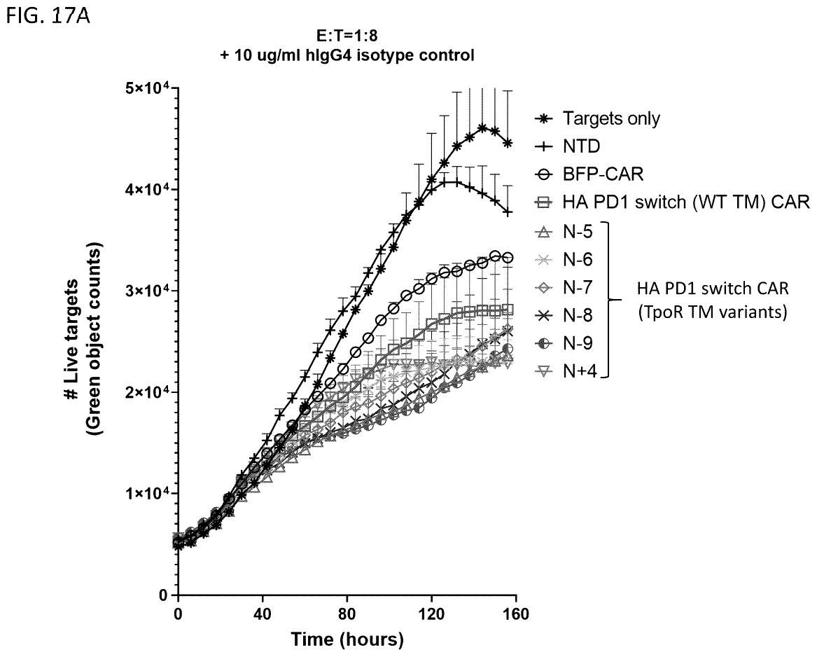

FIG. 16 shows the cytotoxic activity of HA PD1 switch CAR T cells with the indicated TpoR TM domains at an E:T ratio of 1:4 and in the absence of anti-PD-1. As expected, compared to the control BFP-CAR T cells, HA PD1 switch CAR T cells with the WT TpoR TM domain showed improved target cell lysis in vitro. However, cytotoxic activity was further enhanced in HA PD1 switch CAR T cell variants bearing the N−5, N−7, N−8, N−9 or N+4 TpoR TM domains. Concordant with FIGS. 11 and 12, these data indicate that TpoR TM domain variants allow more effective cytotail activation in response to PD-1 ligands, thereby further enhancing the cytotoxic potency of HA PD1 switch CAR T cells.

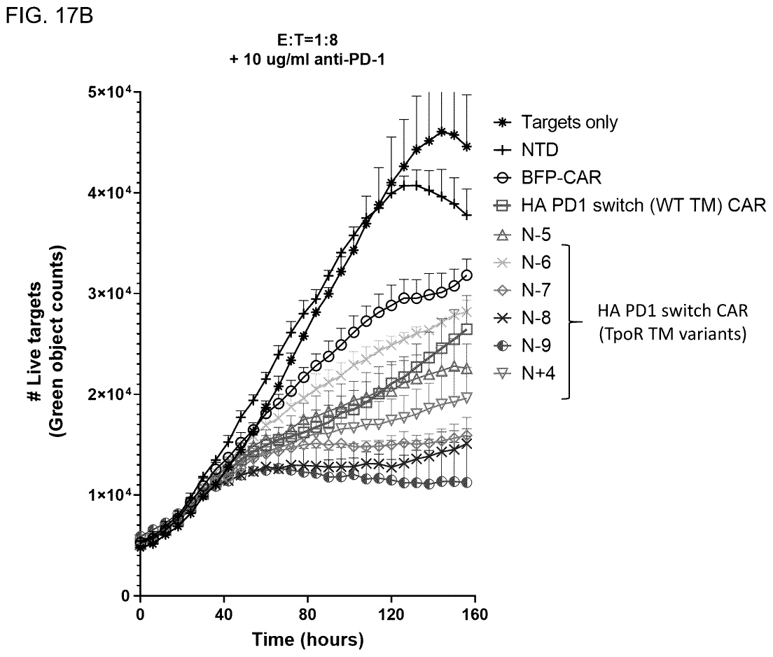

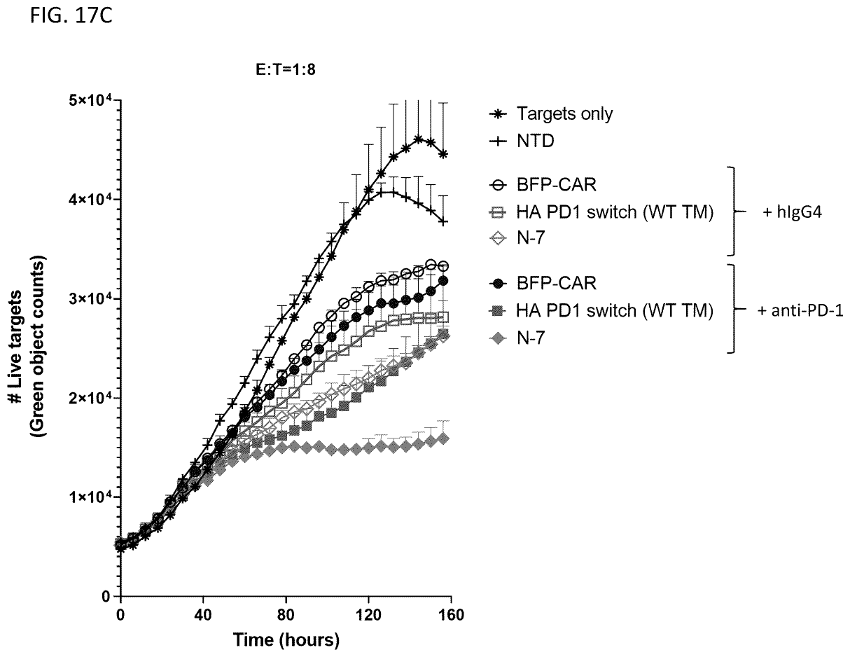

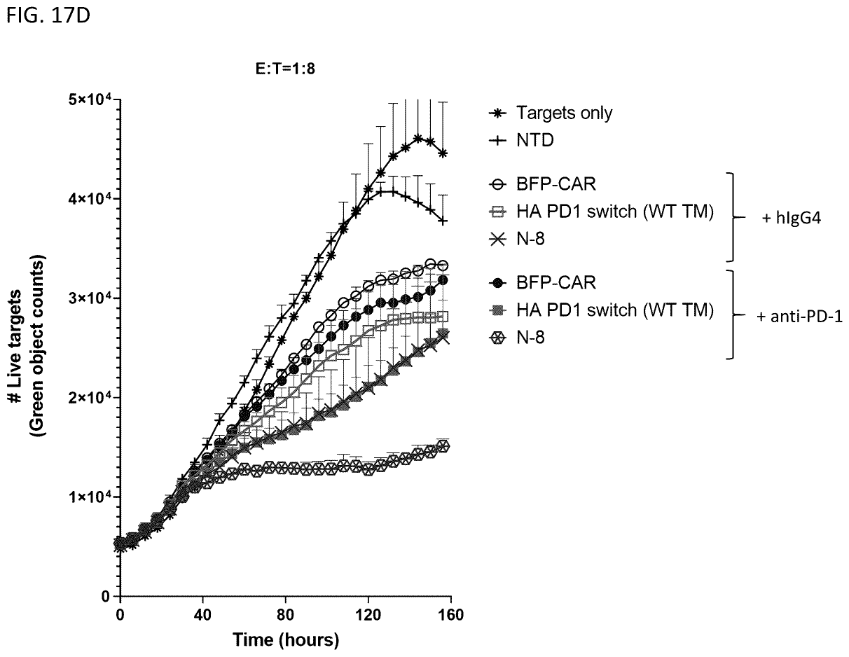

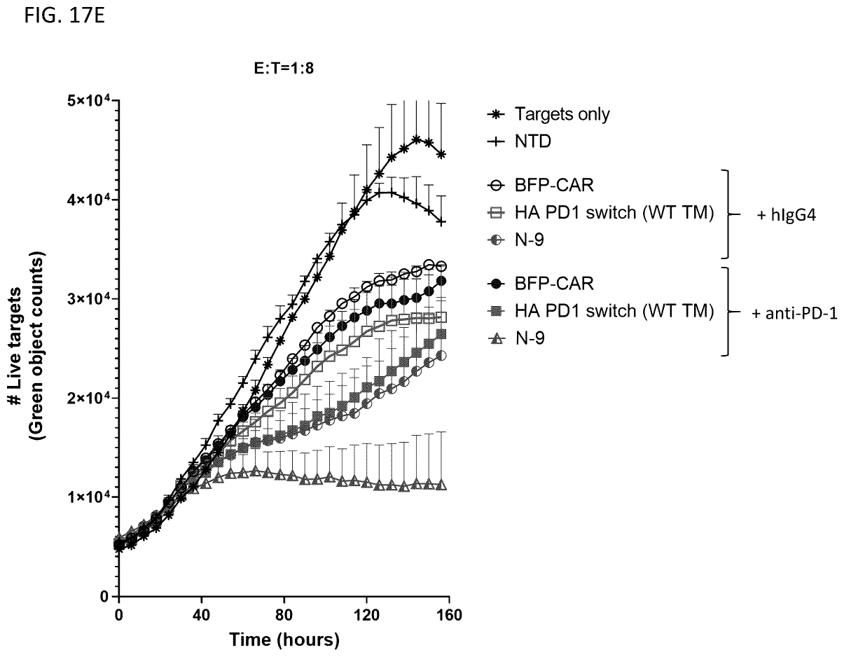

FIGS. 17A-B show the cytotoxic activity of HA PD1 switch CAR T cells with the indicated TpoR TM domains at an E:T ratio of 1:8, and in the absence or presence of anti-PD-1. FIG. 17A shows HA PD1 switch CAR T cell cytotoxicity in the absence of anti-PD-1. As expected, target cell lysis was less efficient at this lower E:T ratio as compared to the 1:4 ratio. Compared to the control BFP-CAR T cells, co-expression of a HA PD1 switch improved CAR T cell cytotoxicity, with the TpoR TM variants showing better activity than their WT TpoR TM counterpart. FIG. 17B shows HA PD1 switch CAR T cell cytotoxicity in the presence of anti-PD-1. Cytotoxic activity of control BFP-CAR T cells was not markedly improved even when combined with anti-PD-1. Additional dimerization and activation by anti-PD-1 improved the activity of HA PD1 switch with WT TpoR TM domain, and this was further enhanced in the N−7, N−8 and N−9 TpoR TM variants. FIGS. 17C-E show a comparison of the N−7 (FIG. 17C), N−8 (FIG. 17D) and N−9 (FIG. 17E) TpoR TM variants in the absence or presence of anti-PD-1. Without being limited to any specific mechanisms, the engagement of the PD-1 ectodomain with monomeric PD-L1/2 expressed on tumor cells or with a dimeric anti-PD-1 antibody may affect the conformation of the TpoR TM helix and different strength of signal transduction. Taken together, increasing the sensitivity and responsiveness of the HA PD1 switch via TpoR TM modifications can markedly improve the efficiency of cytotail signaling and enhance CAR T cell cytotoxic potency.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A PD-1 chimeric cytokine receptor comprising:
a. a PD-1 ectodomain or a PD-L1 antigen binding domain of an anti-PD-L1 or an anti-PD-L2 antibody;
b. a transmembrane domain and a Janus Kinase (JAK)-binding domain comprising SEQ ID NO: 40, SEQ ID NO: 23, SEQ ID NO: 24, SEQ ID NO: 25, SEQ ID NO: 26, or SEQ ID NO: 27; and
c. a STAT recruiting domain comprising a STAT-recruiting domain from a receptor selected from the group consisting of BLNK, IL2RG, EGFR, EpoR, GHR, IFNAR1, IFNAR2, IFNAR1/2, IFNLR1, IL10R1, IL12Rb1, IL12Rb2, IL21R, IL2small, IL7R, IL7Ra, IL9R, IL15R, and IL21R.

2. The chimeric cytokine receptor of claim 1, wherein the STAT-recruiting domain comprises the sequence selected from SEQ ID NOs: 45-55, 57-71 and 78-87.

3. The PD-1 chimeric cytokine receptor of claim 1, wherein the PD-1 ectodomain comprises a wild type PD-1 ectodomain sequence.

4. The PD-1 chimeric cytokine receptor of claim 1, wherein the PD-1 ectodomain comprises one or more mutations to the wild type PD-1 ectodomain sequence.

5. The chimeric cytokine receptor of claim 4, wherein the PD-1 ectodomain comprising one or more mutations has a higher binding affinity for its ligand than a wild type PD-1 ectodomain.

6. The PD-1 chimeric cytokine receptor of claim, wherein the PD-1 ectodomain comprises an amino acid sequence selected from SEQ ID NOs: 2-5, 132-133, and 168-169.

7. The chimeric cytokine receptor of claim 1, wherein the PD-L1 antigen binding domain is an scFv.

8. The chimeric cytokine receptor of claim 1, wherein the chimeric cytokine receptor is activated when the PD-1 ectodomain is bound to PD-L1 or PD-L2.

9. The chimeric cytokine receptor of claim 1, wherein the chimeric cytokine receptor is activated when the PD-1 ectodomain is bound to an anti-PD-1 antibody.

10. The chimeric cytokine receptor of claim 9, wherein the anti-PD-1 antibody is nivolumab or pembrolizumab.

11. The chimeric cytokine receptor of claim 1, wherein the receptor is constitutively active and can be further induced.


SEQUENCE LISTING (224 sequences)

SEQ ID NO: 1 - protein, 21 aa, synthetic construct
  REGION 1..21: note = Description of Artificial Sequence: Synthetic peptide
MALPVTALLLPLALLLHAARP

SEQ ID NO: 2 - protein, 187 aa, unidentified
  REGION 1..187: note = Description of Unknown: PD1 ectodomain sequence
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQ

SEQ ID NO: 3 - protein, 374 aa, synthetic construct
  REGION 1..374: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQ

SEQ ID NO: 4 - protein, 561 aa, synthetic construct
  REGION 1..561: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQ

SEQ ID NO: 5 - protein, 187 aa, unidentified
  REGION 1..187: note = Description of Unknown: High-affinity (HA) PD1 ectodomain sequence
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQ

SEQ ID NO: 6 - protein, 108 aa, synthetic construct
  REGION 1..108: note = Description of Artificial Sequence: Synthetic polypeptide
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 7 - protein, 187 aa, unidentified; identical to SEQ ID NO: 2
  REGION 1..187: note = Description of Unknown: Dominant negative PD1 (21-207) sequence

SEQ ID NO: 8 - protein, 187 aa, unidentified; identical to SEQ ID NO: 5
  REGION 1..187: note = Description of Unknown: Dominant negative high affinity PD1 sequence

SEQ ID NO: 9 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 10 - protein, 918 aa, Homo sapiens [sequence omitted]

SEQ ID NO: 11 - protein, 622 aa, Homo sapiens
MKENVASATVFTLLLFLNTCLLNGQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDTTVWISVAVLSAVICLIIVWAVALKGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 12 - protein, 638 aa, Homo sapiens
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 13 - protein, 863 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEELPGPRQGQWLGQTSEMSRALTPHPCVQDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 14 - protein, 97 aa, unidentified
  REGION 1..97: note = Description of Unknown: GCSFR(614-710) sequence
LTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWE

SEQ ID NO: 15 - protein, 92 aa, Homo sapiens
TTPKFAQGEIEAIVVPVCLAFLLTTLLGVLFCFNKRDLIKKHIWPNVPDPSKSHIAQWSPHTPPRHNFNSKDQMYSDGNFTDVSVVEIEAND

SEQ ID NO: 16 - protein, 105 aa, Homo sapiens
SDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 17 - protein, 104 aa, synthetic construct
  REGION 1..104: note = Description of Artificial Sequence: Synthetic polypeptide
SDPTRVETATETWISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 18 - protein, 103 aa, synthetic construct
  REGION 1..103: note = Description of Artificial Sequence: Synthetic polypeptide
SDPTRVETATETISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 19 - protein, 104 aa, synthetic construct
  REGION 1..104: note = Description of Artificial Sequence: Synthetic polypeptide
SDPTRVETATETLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 20 - protein, 102 aa, synthetic construct
  REGION 1..102: note = Description of Artificial Sequence: Synthetic polypeptide
SDPTRVETATETSLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 21 - protein, 101 aa, synthetic construct
  REGION 1..101: note = Description of Artificial Sequence: Synthetic polypeptide
SDPTRVETATETLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 22 - protein, 102 aa, synthetic construct
  REGION 1..102: note = Description of Artificial Sequence: Synthetic polypeptide
SDPTRVETATETILVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 23 - protein, 100 aa, synthetic construct
  REGION 1..100: note = Description of Artificial Sequence: Synthetic polypeptide
SDPTRVETATETVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 24 - protein, 99 aa, synthetic construct
  REGION 1..99: note = Description of Artificial Sequence: Synthetic polypeptide
SDPTRVETATETTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 25 - protein, 98 aa, synthetic construct
  REGION 1..98: note = Description of Artificial Sequence: Synthetic polypeptide
SDPTRVETATETALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 26 - protein, 97 aa, synthetic construct
  REGION 1..97: note = Description of Artificial Sequence: Synthetic polypeptide
SDPTRVETATETLHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 27 - protein, 96 aa, synthetic construct
  REGION 1..96: note = Description of Artificial Sequence: Synthetic polypeptide
SDPTRVETATETHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 28 - protein, 95 aa, synthetic construct
  REGION 1..95: note = Description of Artificial Sequence: Synthetic polypeptide
SDPTRVETATETLVLGLSAVLGLLLLRWQFPAHYRRLRHALWPSLPDLHRVLGQYLRDTAALSPPKATVSDTCEEVEPSLLEILPKSSERTPLPL

SEQ ID NO: 29 - protein, 94 aa, synthetic construct
  REGION 1..94: note = Description of Artificial Sequence: Synthetic polypeptide
SDPTRVETATETVLGLSAVLGLLLLRWQFPAHYRRLRHALWPSLPDLHRVLGQYLRDTAALSPPKATVSDTCEEVEPSLLEILPKSSERTPLPL

SEQ ID NO: 30 - protein, 93 aa, synthetic construct
  REGION 1..93: note = Description of Artificial Sequence: Synthetic polypeptide
SDPTRVETATETLGLSAVLGLLLLRWQFPAHYRRLRHALWPSLPDLHRVLGQYLRDTAALSPPKATVSDTCEEVEPSLLEILPKSSERTPLPL

SEQ ID NO: 31 - protein, 92 aa, synthetic construct
  REGION 1..92: note = Description of Artificial Sequence: Synthetic polypeptide
SDPTRVETATETGLSAVLGLLLLRWQFPAHYRRLRHALWPSLPDLHRVLGQYLRDTAALSPPKATVSDTCEEVEPSLLEILPKSSERTPLPL

SEQ ID NO: 32 - protein, 91 aa, synthetic construct
  REGION 1..91: note = Description of Artificial Sequence: Synthetic polypeptide
SDPTRVETATETLSAVLGLLLLRWQFPAHYRRLRHALWPSLPDLHRVLGQYLRDTAALSPPKATVSDTCEEVEPSLLEILPKSSERTPLPL

SEQ ID NO: 33 - protein, 90 aa, synthetic construct
  REGION 1..90: note = Description of Artificial Sequence: Synthetic polypeptide
SDPTRVETATETSAVLGLLLLRWQFPAHYRRLRHALWPSLPDLHRVLGQYLRDTAALSPPKATVSDTCEEVEPSLLEILPKSSERTPLPL

SEQ ID NO: 34 - protein, 89 aa, synthetic construct
  REGION 1..89: note = Description of Artificial Sequence: Synthetic polypeptide
SDPTRVETATETAVLGLLLLRWQFPAHYRRLRHALWPSLPDLHRVLGQYLRDTAALSPPKATVSDTCEEVEPSLLEILPKSSERTPLPL

SEQ ID NO: 35 - protein, 88 aa, synthetic construct
  REGION 1..88: note = Description of Artificial Sequence: Synthetic polypeptide
SDPTRVETATETVLGLLLLRWQFPAHYRRLRHALWPSLPDLHRVLGQYLRDTAALSPPKATVSDTCEEVEPSLLEILPKSSERTPLPL

SEQ ID NO: 36 - protein, 87 aa, synthetic construct
  REGION 1..87: note = Description of Artificial Sequence: Synthetic polypeptide
SDPTRVETATETLGLLLLRWQFPAHYRRLRHALWPSLPDLHRVLGQYLRDTAALSPPKATVSDTCEEVEPSLLEILPKSSERTPLPL

SEQ ID NO: 37 - protein, 106 aa, synthetic construct
  REGION 1..106: note = Description of Artificial Sequence: Synthetic polypeptide
SDPTRVETATETAWLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 38 - protein, 107 aa, synthetic construct
  REGION 1..107: note = Description of Artificial Sequence: Synthetic polypeptide
SDPTRVETATETAWVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 39 - protein, 108 aa, synthetic construct
  REGION 1..108: note = Description of Artificial Sequence: Synthetic polypeptide
SDPTRVETATETAWLVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 40 - protein, 109 aa, synthetic construct
  REGION 1..109: note = Description of Artificial Sequence: Synthetic polypeptide
SDPTRVETATETAWILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 41 - protein, 110 aa, synthetic construct
  REGION 1..110: note = Description of Artificial Sequence: Synthetic polypeptide
SDPTRVETATETAWLILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 42 - protein, 111 aa, synthetic construct
  REGION 1..111: note = Description of Artificial Sequence: Synthetic polypeptide
SDPTRVETATETAWLLILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 43 - protein, 112 aa, synthetic construct
  REGION 1..112: note = Description of Artificial Sequence: Synthetic polypeptide
SDPTRVETATETAWVLLILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 44 - protein, 113 aa, synthetic construct
  REGION 1..113: note = Description of Artificial Sequence: Synthetic polypeptide
SDPTRVETATETAWLVLLILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 45 - protein, 144 aa, unidentified
  REGION 1..144: note = Description of Unknown: IL7R(316-459) sequence
ARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 46 - protein, 219 aa, unidentified
  REGION 1..219: note = Description of Unknown: IL2Rb(333-551) sequence
VTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 47 - protein, 50 aa, unidentified
  REGION 1..50: note = Description of Unknown: IFNAR1(508-557) sequence
ISTIATVEETNQTDEDHKKYSSQTSQDSGNYSNEDESESKTSEELQQDFV

SEQ ID NO: 48 - protein, 206 aa, unidentified
  REGION 1..206: note = Description of Unknown: IFNAR2(310-515) sequence
KKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 49 - protein, 256 aa, synthetic construct
  REGION 1..256: note = Description of Artificial Sequence: Synthetic polypeptide
ISTIATVEETNQTDEDHKKYSSQTSQDSGNYSNEDESESKTSEELQQDFVKKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 50 - protein, 221 aa, unidentified
  REGION 1..221: note = Description of Unknown: IFNLR1(300-520) sequence
RGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 51 - protein, 35 aa, unidentified
  REGION 1..35: note = Description of Unknown: Common Gamma Chain(335-369) sequence
IPPKGGALGEGPGASPCNQHSPYWAPPCYTLKPET

SEQ ID NO: 52 - protein, 166 aa, unidentified
  REGION 1..166: note = Description of Unknown: IL9R(356-521) sequence
TALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 53 - protein, 217 aa, unidentified
  REGION 1..217: note = Description of Unknown: IL21R(322-538) sequence
PRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 54 - protein, 286 aa, Homo sapiens
PDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 55 - protein, 170 aa, Homo sapiens
WGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 56 - protein, 203 aa, Mus musculus
AVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 57 - protein, 144 aa, Mus musculus
ARDEVESFLPNDLPAQPEELETQGHRAAVHSANRSPETSVSPPETVRRESPLRCLARNLSTCNAPPLLSSRSPDYRDGDRNRPPVYQDLLPNSGNTNVPVPVPQPLPFQSGILIPVSQRQPISTSSVLNQEEAYVTMSSFYQNK

SEQ ID NO: 58 - protein, 231 aa, unidentified
  REGION 1..231: note = Description of Unknown: EGFR(955-1186) sequence
VIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 59 - protein, 218 aa, synthetic construct
  REGION 1..218: note = Description of Artificial Sequence: Synthetic polypeptide
VIQGDERMHLPSPTDSNFFRALMDEEDMDDVVDADEYLIPQQGFFSSPSTSRTPLLSSLSATSNNSTVACIDRNGLQSCPIKEDSFLQRIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 60 - protein, 54 aa, synthetic construct
  REGION 1..54: note = Description of Artificial Sequence: Synthetic polypeptide
VIQGDERMHLPSPTDSNFFRALMDEEDMDDVVDADEYLIPQQGFFSSPSTSRTP

SEQ ID NO: 61 - protein, 54 aa, unidentified
  REGION 1..54: note = Description of Unknown: EGFR(1019-1085) sequence
NNSTVACIDRNGLQSCPIKEDSFLQRIDDTFLPVPEYINQSVPKRPAGSVQNPV

SEQ ID NO: 62 - protein, 54 aa, synthetic construct
  REGION 1..54: note = Description of Artificial Sequence: Synthetic polypeptide
KEDSFLQRIDDTFLPVPEFINQSVPKRPAGSVQNPVYHNQPLNPAPSRDPHFQD

SEQ ID NO: 63 - protein, 54 aa, synthetic construct
  REGION 1..54: note = Description of Artificial Sequence: Synthetic polypeptide
VPEFINQSVPKRPAGSVQNPVFHNQPLNPAPSRDPHYQDPHSTAVGNPEYLNTV

SEQ ID NO: 64 - protein, 54 aa, unidentified
  REGION 1..54: note = Description of Unknown: EGFR(1122-1165) sequence
PEYLNTVQPTCVNSTFDSPAHWAQKGSHQISLDNPDYQQDFFPKEAKPNGIFKG

SEQ ID NO: 65 - protein, 54 aa, synthetic construct
  REGION 1..54: note = Description of Artificial Sequence: Synthetic polypeptide
WAQKGSHQISLDNPDFQQDFFPKEAKPNGIFKGSTAENAEYLRVAPQSSEFIGA

SEQ ID NO: 66 - protein, 51 aa, unidentified
  REGION 1..51: note = Description of Unknown: IL12Rb2(775-825) sequence
SDPKPENPACPWTVLPAGDLPTHDGYLPSNIDDLPSHEAPLADSLEELEPQ

SEQ ID NO: 67 - protein, 41 aa, unidentified
  REGION 1..41: note = Description of Unknown: IL7R(376-416) sequence
ACDAPILSSSRSLDCRESGKNGPHVYQDLLLSLGTTNSTLP

SEQ ID NO: 68 - protein, 36 aa, unidentified
  REGION 1..36: note = Description of Unknown: IL7R(424-459) sequence
GILTLNPVAQGQPILTSLGSNQEEAYVTMSSFYQNQ

SEQ ID NO: 69 - protein, 68 aa, synthetic construct
  REGION 1..68: note = Description of Artificial Sequence: Synthetic polypeptide
ACDAPILSSSRSLDCRESGKNGPHVYQDLLLSLGTTNSTLPQGQPILTSLGSNQEEAYVTMSSFYQNQ

SEQ ID NO: 70 - protein, 36 aa, synthetic construct
  REGION 1..36: note = Description of Artificial Sequence: Synthetic polypeptide
GILTLNPVAQGQPILTSLGSNQEEAYVTMSSFFQNQ

SEQ ID NO: 71 - protein, 68 aa, synthetic construct
  REGION 1..68: note = Description of Artificial Sequence: Synthetic polypeptide
ACDAPILSSSRSLDCRESGKNGPHVYQDLLLSLGTTNSTLPQGQPILTSLGSNQEEAYVTMSSFFQNQ

SEQ ID NO: 72 - protein, 75 aa, unidentified
  REGION 1..75: note = Description of Unknown: IL2small(393-433) sequence
DEGVAGAPTGSSPQPLQPLSGEDDAYCTFPSRDDLLLFSPSGQGEFRALNARLPLNTDAYLSLQELQGQDPTHLV

SEQ ID NO: 73 - protein, 34 aa, unidentified
  REGION 1..34: note = Description of Unknown: IL2small(518-551) sequence
GQGEFRALNARLPLNTDAYLSLQELQGQDPTHLV

SEQ ID NO: 74 - protein, 82 aa, synthetic construct
  REGION 1..82: note = Description of Artificial Sequence: Synthetic polypeptide
QQDKVPEPASLSSNHSLTSCFTNQGYFFFHLPDALEIEACQDEGVAGAPTGSSPQPLQPLSGEDDAYCTFPSRDDLLLFSPS

SEQ ID NO: 75 - protein, 75 aa, synthetic construct
  REGION 1..75: note = Description of Artificial Sequence: Synthetic polypeptide
QQDKVPEPASLSSNHSLTSCFTNQGYFFFHLPDALEIEACQGQGEFRALNARLPLNTDAYLSLQELQGQDPTHLV

SEQ ID NO: 76 - protein, 75 aa, synthetic construct; identical to SEQ ID NO: 72
  REGION 1..75: note = Description of Artificial Sequence: Synthetic polypeptide

SEQ ID NO: 77 - protein, 116 aa, synthetic construct
  REGION 1..116: note = Description of Artificial Sequence: Synthetic polypeptide
QQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 78 - protein, 43 aa, unidentified
  REGION 1..43: note = Description of Unknown: IFNAR2small(310-352) sequence
KKKVWDYNYDDESDSDTEAAPRTSGGGYTMHGLTVRPLGQASA

SEQ ID NO: 79 - protein, 29 aa, unidentified
  REGION 1..29: note = Description of Unknown: IFNAR2small(486-515) sequence
EGLWSEDAPSDQSDTSESDVDLGDGYIMR

SEQ ID NO: 80 - protein, 72 aa, synthetic construct
  REGION 1..72: note = Description of Artificial Sequence: Synthetic polypeptide
KKKVWDYNYDDESDSDTEAAPRTSGGGYTMHGLTVRPLGQASAEGLWSEDAPSDQSDTSESDVDLGDGYIMR

SEQ ID NO: 81 - protein, 156 aa, unidentified
  REGION 1..156: note = Description of Unknown: BLNK(53-208) sequence
ASESPADEEEQWSDDFDSDYENPDEHSDSEMYVMPAEENADDSYEPPPVEQETRPVHPALPFARGEYIDNRSSQRHSPPFSKTLPSKPSWPSEKARLTSTLPALTALQKPQVPPKPKGLLEDEADYVVPVEDNDENYIHPTESSSPPPEKAPMVNR

SEQ ID NO: 82 - protein, 156 aa, synthetic construct
  REGION 1..156: note = Description of Artificial Sequence: Synthetic polypeptide
ASESPADEEEQWSDDFDSDFENPDEHSDSEMYVMPAEENADDSYEPPPVEQETRPVHPALPFARGEYIDNRSSQRHSPPFSKTLPSKPSWPSEKARLTSTLPALTALQKPQVPPKPKGLLEDEADYVVPVEDNDENYIHPTESSSPPPEKAPMVNR

SEQ ID NO: 83 - protein, 156 aa, synthetic construct
  REGION 1..156: note = Description of Artificial Sequence: Synthetic polypeptide
ASESPADEEEQWSDDFDSDFENPDEHSDSEMYVMPAEENADDSFEPPPVEQETRPVHPALPFARGEYIDNRSSQRHSPPFSKTLPSKPSWPSEKARLTSTLPALTALQKPQVPPKPKGLLEDEADYVVPVEDNDENYIHPTESSSPPPEKAPMVNR

SEQ ID NO: 84 - protein, 170 aa, Homo sapiens; identical to SEQ ID NO: 55

SEQ ID NO: 85 - protein, 149 aa, unidentified
  REGION 1..149: note = Description of Unknown: IL12Rb2(714-862) sequence
VTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 86 - protein, 40 aa, unidentified
  REGION 1..40: note = Description of Unknown: IL12Rb1(622-662) sequence
WDKGERTEPLEKTELPEGAPELALDTELSLEDGDRCKAKM

SEQ ID NO: 87 - protein, 275 aa, unidentified
  REGION 1..275: note = Description of Unknown: IL10R1(304-578) sequence
VSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 88 - protein, 487 aa, synthetic construct
  REGION 1..487: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 89 - protein, 479 aa, synthetic construct
  REGION 1..479: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 90 - protein, 423 aa, synthetic construct
  REGION 1..423: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 91 - protein, 674 aa, synthetic construct
  REGION 1..674: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 92 - protein, 861 aa, synthetic construct
  REGION 1..861: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 93 - protein, 487 aa, synthetic construct
  REGION 1..487: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 94 - protein, 486 aa, synthetic construct
  REGION 1..486: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETWISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 95 - protein, 485 aa, synthetic construct
  REGION 1..485: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 96 - protein, 486 aa, synthetic construct
  REGION 1..486: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 97 - protein, 484 aa, synthetic construct
  REGION 1..484: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETSLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 98 - protein, 483 aa, synthetic construct
  REGION 1..483: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 99 - protein, 484 aa, synthetic construct
  REGION 1..484: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETILVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 100 - protein, 482 aa, synthetic construct
  REGION 1..482: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 101 - protein, 481 aa, synthetic construct
  REGION 1..481: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 102 - protein, 480 aa, synthetic construct
  REGION 1..480: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 103 - protein, 479 aa, synthetic construct
  REGION 1..479: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETLHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 104 - protein, 478 aa, synthetic construct
  REGION 1..478: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 105 - protein, 477 aa, synthetic construct
  REGION 1..477: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 106 - protein, 476 aa, synthetic construct
  REGION 1..476: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 107 - protein, 475 aa, synthetic construct
  REGION 1..475: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 108 - protein, 474 aa, synthetic construct
  REGION 1..474: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 109 - protein, 473 aa, synthetic construct
  REGION 1..473: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 110 - protein, 472 aa, synthetic construct
  REGION 1..472: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 111 - protein, 471 aa, synthetic construct
  REGION 1..471: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 112 - protein, 470 aa, synthetic construct
  REGION 1..470: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 113 - protein, 469 aa, synthetic construct
  REGION 1..469: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 114 - protein, 488 aa, synthetic construct
  REGION 1..488: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETAWLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 115 - protein, 489 aa, synthetic construct
  REGION 1..489: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETAWVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 116 - protein, 490 aa, synthetic construct
  REGION 1..490: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETAWLVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 117 - protein, 491 aa, synthetic construct
  REGION 1..491: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETAWILVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 118 - protein, 492 aa, synthetic construct
  REGION 1..492: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETAWLILVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 119 - protein, 493 aa, synthetic construct
  REGION 1..493: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETAWLLILVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 120 - protein, 494 aa, synthetic construct
  REGION 1..494: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETAWVLLILVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 121 - protein, 495 aa, synthetic construct
  REGION 1..495: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQSDPTRVETATETAWLVLLILVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 122 - protein, 11 aa, synthetic construct
  REGION 1..11: note = Description of Artificial Sequence: Synthetic peptide
IPNPLLGLDST

SEQ ID NO: 123 - protein, 246 aa, synthetic construct
  REGION 1..246: note = Description of Artificial Sequence: Synthetic polypeptide
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 124 - protein, 69 aa, synthetic construct
  REGION 1..69: note = Description of Artificial Sequence: Synthetic polypeptide
TTTPAPRPPTPAPTIASQPLSLRPEACRPAAGGAVHTRGLDFACDIYIWAPLAGTCGVLLLSLVITLYC

SEQ ID NO: 125 - protein, 42 aa, synthetic construct
  REGION 1..42: note = Description of Artificial Sequence: Synthetic polypeptide
KRGRKKLLYIFKQPFMRPVQTTQEEDGCSCRFPEEEEGGCEL

SEQ ID NO: 126 - protein, 112 aa, synthetic construct
  REGION 1..112: note = Description of Artificial Sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 233 aa, synthetic construct
  REGION 1..233: note = Description of Artificial Sequence: Synthetic polypeptide
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 128 - protein, 22 aa, synthetic construct
  REGION 1..22: note = Description of Artificial Sequence: Synthetic peptide
GSGATNFSLLKQAGDVEENPGP

SEQ ID NO: 129 - protein, 208 aa, synthetic construct
  REGION 1..208: note = Description of Artificial Sequence: Synthetic polypeptide
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQ

SEQ ID NO: 130 - protein, 208 aa, synthetic construct
  REGION 1..208: note = Description of Artificial Sequence: Synthetic polypeptide
MALPVTALLLPLALLLHAARPPGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQ

SEQ ID NO: 131 - protein, 635 aa, Homo sapiens
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 132 - protein, 150 aa, unidentified [sequence omitted]

SEQ ID NO: 133 - protein, 150 aa, unidentified
  REGION 1..150: note = Description of Unknown: High-affinity (HA) PD1 ectodomain sequence
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLV

SEQ ID NO: 134 - protein, 105 aa, synthetic construct
  REGION 1..105: note = Description of Artificial Sequence: Synthetic polypeptide
SDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPL

SEQ ID NO: 135 - protein, 105 aa, synthetic construct
  REGION 1..105: note = Description of Artificial Sequence: Synthetic polypeptide
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPL

SEQ ID NO: 136 - protein, 399 aa, synthetic construct
  REGION 1..399: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 137 - protein, 330 aa, synthetic construct
  REGION 1..330: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLDEGVAGAPTGSSPQPLQPLSGEDDAYCTFPSRDDLLLFSPSGQGEFRALNARLPLNTDAYLSLQELQGQDPTHLV

SEQ ID NO: 138 - protein, 187 aa, synthetic construct; identical to SEQ ID NO: 5
  REGION 1..187: note = Description of Artificial Sequence: Synthetic polypeptide

SEQ ID NO: 139 - protein, 399 aa, synthetic construct
  REGION 1..399: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 140 - protein, 330 aa, synthetic construct
  REGION 1..330: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLDEGVAGAPTGSSPQPLQPLSGEDDAYCTFPSRDDLLLFSPSGQGEFRALNARLPLNTDAYLSLQELQGQDPTHLV

SEQ ID NO: 141 - protein, 420 aa, synthetic construct
  REGION 1..420: note = Description of Artificial Sequence: Synthetic polypeptide
MALPVTALLLPLALLLHAARPPGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 142 - protein, 351 aa, synthetic construct
  REGION 1..351: note = Description of Artificial Sequence: Synthetic polypeptide
MALPVTALLLPLALLLHAARPPGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLDEGVAGAPTGSSPQPLQPLSGEDDAYCTFPSRDDLLLFSPSGQGEFRALNARLPLNTDAYLSLQELQGQDPTHLV

SEQ ID NO: 143 - protein, 208 aa, synthetic construct; identical to SEQ ID NO: 130
  REGION 1..208: note = Description of Artificial Sequence: Synthetic polypeptide

SEQ ID NO: 144 - protein, 420 aa, synthetic construct
  REGION 1..420: note = Description of Artificial Sequence: Synthetic polypeptide
MALPVTALLLPLALLLHAARPPGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 145 - protein, 351 aa, synthetic construct
  REGION 1..351: note = Description of Artificial Sequence: Synthetic polypeptide
MALPVTALLLPLALLLHAARPPGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLDEGVAGAPTGSSPQPLQPLSGEDDAYCTFPSRDDLLLFSPSGQGEFRALNARLPLNTDAYLSLQELQGQDPTHLV

SEQ ID NO: 146 - protein, 55 aa, Homo sapiens
SDPTRVETATETAWISLVTALHLVLGLSAVLGLLLLRWQFPAHYRRLRHALWPSL

SEQ ID NO: 147 - protein, 54 aa, synthetic construct
  REGION 1..54: note = Description of Artificial Sequence: Synthetic polypeptide
SDPTRVETATETWISLVTALHLVLGLSAVLGLLLLRWQFPAHYRRLRHALWPSL

SEQ ID NO: 148 - protein, 53 aa, synthetic construct
  REGION 1..53: note = Description of Artificial Sequence: Synthetic polypeptide
SDPTRVETATETISLVTALHLVLGLSAVLGLLLLRWQFPAHYRRLRHALWPSL

SEQ ID NO: 149 - protein, 54 aa, synthetic construct
  REGION 1..54: note = Description of Artificial Sequence: Synthetic polypeptide
SDPTRVETATETLISLVTALHLVLGLSAVLGLLLLRWQFPAHYRRLRHALWPSL

SEQ ID NO: 150 - protein, 52 aa, synthetic construct
  REGION 1..52: note = Description of Artificial Sequence: Synthetic polypeptide
SDPTRVETATETSLVTALHLVLGLSAVLGLLLLRWQFPAHYRRLRHALWPSL

SEQ ID NO: 151 - protein, 51 aa, synthetic construct
  REGION 1..51: note = Description of Artificial Sequence: Synthetic polypeptide
SDPTRVETATETLVTALHLVLGLSAVLGLLLLRWQFPAHYRRLRHALWPSL

SEQ ID NO: 152 - protein, 52 aa, synthetic construct
  REGION 1..52: note = Description of Artificial Sequence: Synthetic polypeptide
SDPTRVETATETILVTALHLVLGLSAVLGLLLLRWQFPAHYRRLRHALWPSL

SEQ ID NO: 153 - protein, 50 aa, synthetic construct
  REGION 1..50: note = Description of Artificial Sequence: Synthetic polypeptide
SDPTRVETATETVTALHLVLGLSAVLGLLLLRWQFPAHYRRLRHALWPSL

SEQ ID NO: 154 - protein, 49 aa, synthetic construct
  REGION 1..49: note = Description of Artificial Sequence: Synthetic polypeptide
SDPTRVETATETTALHLVLGLSAVLGLLLLRWQFPAHYRRLRHALWPSL

SEQ ID NO: 155 - protein, 48 aa, synthetic construct
  REGION 1..48: note = Description of Artificial Sequence: Synthetic polypeptide
SDPTRVETATETALHLVLGLSAVLGLLLLRWQFPAHYRRLRHALWPSL

SEQ ID NO: 156 - protein, 47 aa, synthetic construct
  REGION 1..47: note = Description of Artificial Sequence: Synthetic polypeptide
SDPTRVETATETLHLVLGLSAVLGLLLLRWQFPAHYRRLRHALWPSL

SEQ ID NO: 157 - protein, 46 aa, synthetic construct
  REGION 1..46: note = Description of Artificial Sequence: Synthetic polypeptide
SDPTRVETATETHLVLGLSAVLGLLLLRWQFPAHYRRLRHALWPSL

SEQ ID NO: 158 - protein, 55 aa, Homo sapiens; identical to SEQ ID NO: 146

SEQ ID NO: 159 - protein, 56 aa, synthetic construct
  REGION 1..56: note = Description of Artificial Sequence: Synthetic polypeptide
SDPTRVETATETAWLISLVTALHLVLGLSAVLGLLLLRWQFPAHYRRLRHALWPSL

SEQ ID NO: 160 - protein, 57 aa, synthetic construct
  REGION 1..57: note = Description of Artificial Sequence: Synthetic polypeptide
SDPTRVETATETAWVLISLVTALHLVLGLSAVLGLLLLRWQFPAHYRRLRHALWPSL

SEQ ID NO: 161 - protein, 58 aa, synthetic construct
  REGION 1..58: note = Description of Artificial Sequence: Synthetic polypeptide
SDPTRVETATETAWLVLISLVTALHLVLGLSAVLGLLLLRWQFPAHYRRLRHALWPSL

SEQ ID NO: 162 - protein, 59 aa, synthetic construct
  REGION 1..59: note = Description of Artificial Sequence: Synthetic polypeptide
SDPTRVETATETAWILVLISLVTALHLVLGLSAVLGLLLLRWQFPAHYRRLRHALWPSL

SEQ ID NO: 163 - protein, 60 aa, synthetic construct
  REGION 1..60: note = Description of Artificial Sequence: Synthetic polypeptide
SDPTRVETATETAWLILVLISLVTALHLVLGLSAVLGLLLLRWQFPAHYRRLRHALWPSL

SEQ ID NO: 164 - protein, 61 aa, synthetic construct
  REGION 1..61: note = Description of Artificial Sequence: Synthetic polypeptide
SDPTRVETATETAWLLILVLISLVTALHLVLGLSAVLGLLLLRWQFPAHYRRLRHALWPSL

SEQ ID NO: 165 - protein, 62 aa, synthetic construct
  REGION 1..62: note = Description of Artificial Sequence: Synthetic polypeptide
SDPTRVETATETAWVLLILVLISLVTALHLVLGLSAVLGLLLLRWQFPAHYRRLRHALWPSL

SEQ ID NO: 166 - protein, 63 aa, synthetic construct
  REGION 1..63: note = Description of Artificial Sequence: Synthetic polypeptide
SDPTRVETATETAWLVLLILVLISLVTALHLVLGLSAVLGLLLLRWQFPAHYRRLRHALWPSL

SEQ ID NO: 167 - protein, 13 aa, synthetic construct
  REGION 1..13: note = Description of Artificial Sequence: Synthetic peptide
KPIPNPLLGLDST

SEQ ID NO: 168 - protein, 301 aa, synthetic construct
  REGION 1..301: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 169 - protein, 452 aa, synthetic construct
  REGION 1..452: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 170 - protein, 219 aa, synthetic construct
  REGION 1..219: note = Description of Artificial Sequence: Synthetic polypeptide
VTQLLLQQDKVPEPASLSSNHSLTSCFTNQGYFFFHLPDALEIEACQVSFTSDPSSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 171 - protein, 219 aa, synthetic construct
  REGION 1..219: note = Description of Artificial Sequence: Synthetic polypeptide
VTQLLLQQDKVPEPASLSSNHSLTSCFTNQGSFFFHLPDALEIEACQVSFTSDPSSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 172 - protein, 457 aa, synthetic construct
  REGION 1..457: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 173 - protein, 449 aa, synthetic construct
  REGION 1..449: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWELEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 174 - protein, 444 aa, synthetic construct
  REGION 1..444: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 175 - protein, 608 aa, synthetic construct
  REGION 1..608: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 176 - protein, 759 aa, synthetic construct
  REGION 1..759: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 177 - protein, 457 aa, synthetic construct
  REGION 1..457: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 178 - protein, 456 aa, synthetic construct
  REGION 1..456: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETWISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 179 - protein, 455 aa, synthetic construct
  REGION 1..455: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 180 - protein, 456 aa, synthetic construct
  REGION 1..456: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 181 - protein, 454 aa, synthetic construct
  REGION 1..454: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETSLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 182 - protein, 453 aa, synthetic construct
  REGION 1..453: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 183 - protein, 454 aa, synthetic construct
  REGION 1..454: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETILVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 184 - protein, 452 aa, synthetic construct
  REGION 1..452: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 185 - protein, 451 aa, synthetic construct
  REGION 1..451: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 186 - protein, 450 aa, synthetic construct
  REGION 1..450: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 187 - protein, 449 aa, synthetic construct
  REGION 1..449: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETLHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 188 - protein, 448 aa, synthetic construct
  REGION 1..448: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 189 - protein, 447 aa, synthetic construct
  REGION 1..447: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 190 - protein, 446 aa, synthetic construct
  REGION 1..446: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 191 - protein, 445 aa, synthetic construct
  REGION 1..445: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 192 - protein, 444 aa, synthetic construct
  REGION 1..444: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 193 - protein, 443 aa, synthetic construct
  REGION 1..443: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 194 - protein, 442 aa, synthetic construct
  REGION 1..442: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 195 - protein, 441 aa, synthetic construct
  REGION 1..441: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 196 - protein, 440 aa, synthetic construct
  REGION 1..440: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 197 - protein, 439 aa, synthetic construct
  REGION 1..439: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 198 - protein, 458 aa, synthetic construct
  REGION 1..458: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 199 - protein, 459 aa, synthetic construct
  REGION 1..459: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 200 - protein, 460 aa, synthetic construct
  REGION 1..460: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWLVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 201 - protein, 461 aa, synthetic construct
  REGION 1..461: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWILVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 202 - protein, 462 aa, synthetic construct
  REGION 1..462: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWLILVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 203 - protein, 463 aa, synthetic construct
  REGION 1..463: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWLLILVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 204 - protein, 464 aa, synthetic construct
  REGION 1..464: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWVLLILVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 205 - protein, 465 aa, synthetic construct
  REGION 1..465: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWLVLLILVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGSRSDPKPENPACPWTVLPAGDLPTHDGYLPSNIDDLPSHEAPLADSLEELEPQ

SEQ ID NO: 206 - protein, 401 aa, synthetic construct
  REGION 1..401: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 207 - protein, 332 aa, synthetic construct
  REGION 1..332: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 208 - protein, 401 aa, synthetic construct
  REGION 1..401: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 209 - protein, 332 aa, synthetic construct
  REGION 1..332: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 210 - protein, 422 aa, synthetic construct
  REGION 1..422: note = Description of Artificial Sequence: Synthetic polypeptide
MALPVTALLLPLALLLHAARPPGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 211 - protein, 353 aa, synthetic construct
  REGION 1..353: note = Description of Artificial Sequence: Synthetic polypeptide
MALPVTALLLPLALLLHAARPPGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 212 - protein, 422 aa, synthetic construct
  REGION 1..422: note = Description of Artificial Sequence: Synthetic polypeptide
MALPVTALLLPLALLLHAARPPGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 213 - protein, 353 aa, synthetic construct
  REGION 1..353: note = Description of Artificial Sequence: Synthetic polypeptide
MALPVTALLLPLALLLHAARPPGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 214 - protein, 373 aa, synthetic construct
  REGION 1..373: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEQQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 215 - protein, 373 aa, synthetic construct
  REGION 1..373: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLEQQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 216 - protein, 373 aa, synthetic construct
  REGION 1..373: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLLEQQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 217 - protein, 325 aa, synthetic construct
  REGION 1..325: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETALHLVLGLSAVLGLLLLRWQFPAHYRRLRHALWPSLPDLHRVLGQYLRDTAALSPPKATVSDTCEEVEPSLLEILPKSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 218 - protein, 324 aa, synthetic construct
  REGION 1..324: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETLHLVLGLSAVLGLLLLRWQFPAHYRRLRHALWPSLPDLHRVLGQYLRDTAALSPPKATVSDTCEEVEPSLLEILPKSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 219 - protein, 323 aa, synthetic construct
  REGION 1..323: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETHLVLGLSAVLGLLLLRWQFPAHYRRLRHALWPSLPDLHRVLGQYLRDTAALSPPKATVSDTCEEVEPSLLEILPKSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 220 - protein, 366 aa, synthetic construct
  REGION 1..366: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETALHLVLGLSAVLGLLLLRWQFPAHYRRLRHALWPSLPDLHRVLGQYLRDTAALSPPKATVSDTCEEVEPSLLEILPKSSERTPLPLLEQQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 221 - protein, 365 aa, synthetic construct
  REGION 1..365: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETLHLVLGLSAVLGLLLLRWQFPAHYRRLRHALWPSLPDLHRVLGQYLRDTAALSPPKATVSDTCEEVEPSLLEILPKSSERTPLPLLEQQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 222 - protein, 364 aa, synthetic construct
  REGION 1..364: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETHLVLGLSAVLGLLLLRWQFPAHYRRLRHALWPSLPDLHRVLGQYLRDTAALSPPKATVSDTCEEVEPSLLEILPKSSERTPLPLLEQQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 223 - protein, 332 aa, synthetic construct
  REGION 1..332: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 224 - protein, 401 aa, synthetic construct
  REGION 1..401: note = Description of Artificial Sequence: Synthetic polypeptide
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ